SEQUENCE LISTING (23 sequences)

SEQ ID NO: 1 - protein, 125 aa, Artificial Sequence
  Synthetic
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 2 - protein, 127 aa, Artificial Sequence
  Synthetic
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGS

SEQ ID NO: 3 - protein, 127 aa, Artificial Sequence
  Synthetic
QVQLQESGGGLVQAGGSLRLSCAASGRTFSEYAMGWFRQAPGREREFVATISWSGGSTYYTDSVRGRFTISRDNARNTVYLQMNSLRPDDTAVYYCAAAGLGTKVSEWDYDYDYWGQGTQVTVSSGS

SEQ ID NO: 4 - protein, 127 aa, Artificial Sequence; identical to SEQ ID NO: 3
  Synthetic

SEQ ID NO: 5 - protein, 159 aa, Artificial Sequence
  Synthetic
MGSSHHHHHHSSGLVPRGSHQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGGSGGGSGGGSG

SEQ ID NO: 6 - protein, 159 aa, Artificial Sequence
  Synthetic
MGSSHHHHHHSSGLVPRGSHQVQLQESGGGLVQAGGSLRLSCAASGRTFSEYAMGWFRQAPGREREFVATISWSGGSTYYTDSVRGRFTISRDNARNTVYLQMNSLRPDDTAVYYCAAAGLGTKVSEWDYDYDYWGQGTQVTVSSGSGGSGGGSGGGSG

SEQ ID NO: 7 - protein, 20 aa, Artificial Sequence
  Synthetic
MGSSHHHHHHSSGLVPRGSH

SEQ ID NO: 8 - protein, 12 aa, Artificial Sequence
  Synthetic
GGSGGGSGGGSG

SEQ ID NO: 9 - protein, 142 aa, Artificial Sequence
  Synthetic
GSHQVQLQESGGGLVQAGGSLRLSCAASGRTFSEYAMGWFRQAPGREREFVATISWSGGSTYYTDSVRGRFTISRDNARNTVYLQMNSLRPDDTAVYYCAAAGLGTKVSEWDYDYDYWGQGTQVTVSSGSGGSGGGSGGGSG

SEQ ID NO: 10 - protein, 127 aa, Artificial Sequence
  Synthetic
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSS

SEQ ID NO: 11 - protein, 127 aa, Artificial Sequence
  Synthetic
QVQLVESGGGLMQAGGSLRLSCAVSGRTKSTAAMGWFRQAPGREREFVAAIRWSGGSAYYADSVRGRFTISRDRARNTVYLQMNSLRYEDTAVYYCAQTHYVSYLLSDYATWPYDYWGQGTQVTVSS

SEQ ID NO: 12 - protein, 120 aa, Artificial Sequence
  Synthetic
VQLQASGGGLVRPGGSLRLSCAASGFTFSSYAMMWVRQAPGKGLEWVSAINGGGGSTSYADSVKGRFTISRDNAKNTLYLQMNSLKPEDTAVYYCAKYQAAVHQEKEDYWGQGTQVTVSS

SEQ ID NO: 13 - protein, 125 aa, Artificial Sequence
  Synthetic
QSSGEVQLQASGGGLVRPGGSLRLSCAASGFTFSSYAMMWVRQAPGRGLEWVSAINGGGGSTSYADSVKGRFTISRDNARNTLYLQMNSLRPEDTAVYYCARYQAAVHQEREDYWGQGTQVTVSS

SEQ ID NO: 14 - protein, 64 aa, Artificial Sequence
  Synthetic
NDDELHMLMTDLVYEALHFAKDEEIKKRVFQLFELADKAYKNNDRQKLEKVVEELKELLERLLS

SEQ ID NO: 15 - protein, 65 aa, Artificial Sequence
  Synthetic
HNDDELHMLMTDLVYEALCFAKDEEIKKRVFQLFELADKAYKNNDRQKLEKVVEELKELLERLLS

SEQ ID NO: 16 - protein, 121 aa, Artificial Sequence
  Synthetic
VQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 17 - protein, 121 aa, Artificial Sequence
  Synthetic
VQLQESGGGLVQPGGSLRLSCTASGVTISALNAMAMGWYRQAPGERRVMVAAVSERGNACYRESVQGRFTVTRDFTNRMVSLQMDNLRPEDTAVYYCHVLEDRVDSFHDYWGQGTQVTVSS

SEQ ID NO: 18 - protein, 127 aa, Artificial Sequence
  Synthetic
QVQLQESGGGSVQAGGSLRLSCVVSGNTGSTGYWAWFRQGPGTEREGVAATYTAGSGTSMTYYADSVKGRFTISQDNAKKTLYLQMNSLKPEDTGMYRCASTRFAGRWYRDSEYRAWGQGTQVTVSS

SEQ ID NO: 19 - protein, 127 aa, Artificial Sequence
  Synthetic
QVQLQESGGGSVQAGGSLRLSCVVSGNTGSTGYWAWFRQGPGTEREGVAACYTAGSGTSMTYYADSVKGRFTISQDNAKKTLYLQMNSLKPEDTGMYRCASTRFAGRWYRDSEYRAWGQGTQVTVSS

SEQ ID NO: 20 - protein, 161 aa, Artificial Sequence
  Synthetic
MGSSHHHHHHSSGLVPRGSHQVQLVESGGGLMQAGGSLRLSCAVSGRTKSTAAMGWFRQAPGREREFVAAIRWSGGSAYYADSVRGRFTISRDRARNTVYLQMNSLRYEDTAVYYCAQTHYVSYLLSDYATWPYDYWGQGTQVTVSSGSGGSGGGSGGGSG

SEQ ID NO: 21 - protein, 156 aa, Artificial Sequence
  Synthetic
MHHHHHHHHSSGENLYFQSSGEVQLQASGGGLVRPGGSLRLSCAASGFTFSSYAMMWVRQAPGRGLEWVSAINGGGGSTSYADSVKGRFTISRDNARNTLYLQMNSLRPEDTAVYYCARYQAAVHQEREDYWGQGTQVTVSSGSGGSGGGSGGGSG

SEQ ID NO: 22 - protein, 157 aa, Artificial Sequence
  Synthetic
MHHHHHHHHSSGENLYFQSSGEVQLQESGGGLVQPGGSLRLSCTASGVTISALNAMAMGWYRQAPGERRVMVAAVSERGNACYRESVQGRFTVTRDFTNRMVSLQMDNLRPEDTAVYYCHVLEDRVDSFHDYWGQGTQVTVSSGSGGSGGGSGGGSG

SEQ ID NO: 23 - protein, 84 aa, Artificial Sequence
  Synthetic
MGSSHHHHHHSSGLVPRGSHNDDELHMLMTDLVYEALCFAKDEEIKKRVFQLFELADKAYKNNDRQKLEKVVEELKELLERLLS

(19) United States
(12) Patent Application Publication
(10) Pub. No.: US 2024/0201179 A1
(43) Pub. Date: Jun. 20, 2024

(54) FLUOROGENIC SENSORS FOR DETECTING ANTIGENS

(71) Applicant: President and Fellows of Harvard College, Cambridge, MA (US)

(72) Inventors: Erkin Kuru, Cambridge, MA (US); George M. Church, Cambridge, MA (US); Janathan Rittichier, Cambridge, MA (US); Isaac Han, Cambridge, MA (US); Helena de Puig Guixe, Cambridge, MA (US)

(73) Assignee: President and Fellows of Harvard College, Cambridge, MA (US)

(21) Appl. No.: 18/282,991

(22) PCT Filed: Mar. 25, 2022
(86) PCT No.: PCT/US2022/021878

§ 371 Date: Sep. 19, 2023

Related U.S. Application Data
(60) Provisional application No. 63/165,934, filed on Mar. 25, 2021.

(51) Int. Cl. G01N 33/543; C07C 251/30; C07D 207/273; C07D 207/337; C07D 471/06; G01N 21/64; G01N 33/569; G01N 33/58

(52) U.S. Cl. CPC G01N 33/54373; C07C 251/30; C07D 207/273; C07D 207/337; C07D 471/06; G01N 21/6408; G01N 21/6428; G01N 33/56911; G01N 33/56983; G01N 33/58; G01N 2021/6439; G01N 2333/165; G01N 2333/34; G01N 2469/10

(57) ABSTRACT

Provided herein are fluorogenic sensors which can be used to detect targets (e.g., antigens). In general, the fluorogenic sensors provided herein comprise a protein (e.g., antibody, nanobody, mini-protein) and a fluorogenic small molecule conjugated to the target-binding domain (e.g., antigen-binding domain) of the protein. Upon binding of the protein to said target (e.g., antigen), the fluorogenic small molecule may increase or decrease in fluorescence or exhibit a change in fluorescence lifetime (i.e., “turn on”), thereby indicating the presence of the target (e.g., antigen).

### RELATED APPLICATIONS

This application claims priority under 35 U.S.C. § 119(e) to U.S. Provisional Patent Application Ser. No. 63/165,934, filed Mar. 25, 2021, the entire contents of which is incorporated herein by reference.

### GOVERNMENT SUPPORT

This invention was made with government support under DE-FG02-02ER6344 awarded by the Department of Energy (DOE). The government has certain rights in the invention.

### BACKGROUND

Fluorescent labeling technologies continue to be on the frontier of research in the life sciences as well as emerging as a powerful tool in diagnostics and industrial applications. However, high background signal originating from fluorescent probes that do not specifically label their target (unreacted and off target labeling) is a big challenge for biochemical labeling.

### SUMMARY OF THE DISCLOSURE

Certain fluorescent molecules are conditionally fluorescent, i.e., “fluorogenic,” because they can selectively “turn on” (e.g., increase/decrease in fluorescence and/or change their fluorescence lifetime) upon the occurrence of a chemical or physical event. Examples of such events include changes in viscosity and local dipole environment (polarity). Both of these changes, viscosity and polarity, can occur at protein-protein binding interfaces (e.g., protein-antigen binding interfaces). In the case of protein-antigen binding, for example, selectively conjugating fluorogenic molecules at or around the binding domains of antigen-binding proteins (e.g., antibodies, nanobodies, mini-proteins, or variants or fragments thereof) can provide fluorogenic sensors that detect protein-antigen binding, e.g., with low background fluorescence or distinct fluorescence lifetime.

Provided herein are fluorogenic sensors which can be used to detect target molecules (e.g., antigens) by sensing protein-target molecule interactions. In general, the fluorogenic sensors provided herein comprise a protein (e.g., antibody, nanobody, mini-protein, designed ankyrin repeat protein (DARPin), monobody) with a fluorogenic small molecule conjugated at or around a target-binding domain of the protein. The protein (e.g., nanobody or mini-protein) may specifically bind said target (e.g., antigen). Upon binding of the protein to said target, the fluorogenic small molecule may increase/decrease in fluorescence (i.e., “turn on”) or detectably change its fluorescence lifetime in response to changes in polarity, viscosity, spatial constraints, or other physical changes. This increase/decrease in fluorescence or change in fluorescence lifetime may be indicative of binding of the protein (e.g., antibody, nanobody) to the target (e.g., antigen), and therefore indicative of the presence of the target (e.g., antigen).

In certain embodiments, the target molecule is an antigen. Therefore, provided herein are fluorogenic sensors which can be used to detect antigens. In certain embodiments, the fluorogenic sensors provided herein comprise a protein (e.g., nanobody or mini-protein) with a fluorogenic small molecule conjugated at or around the antigen-binding domain of the protein. The protein (e.g., nanobody or mini-protein) may specifically bind said antigen. Upon binding of the protein to said antigen, an increase/decrease in fluorescence or change in fluorescence lifetime may be indicative of binding of the protein (e.g., nanobody or mini-protein) to the antigen, and therefore indicative of the presence of the antigen (e.g., in a sample, such as biological sample).

In one aspect, provided herein are fluorogenic sensors for detecting targets (e.g., antigens) comprising: a nanobody, and a fluorogenic small molecule conjugated at or around a target-binding domain (e.g., antigen-binding domain) of the nanobody. In certain embodiments, the nanobody specifically binds a pathogen (e.g., a virus), and therefore the fluorogenic sensor can be used to detect said pathogen. In certain embodiments, the antigen is from a pathogen or present on a pathogen, and therefore the binding the fluorogenic sensor to said antigen is indicative of the presence of said pathogen. For example, in certain embodiments, the nanobody specifically binds a spike protein of a coronavirus or variant thereof, and the fluorogenic sensor can be used to detect said coronavirus or variant thereof. In certain embodiments, the nanobody specifically binds a spike protein of a SARS-CoV-2 virus or variant thereof, and therefore the fluorogenic sensor can be used to detect a SARS-CoV-2 virus or variant thereof.

For example, in certain embodiments, the nanobody comprises a VHH72 nanobody or a fragment thereof. In certain embodiments, the nanobody comprises an amino acid sequence with at least 80% sequence identity with the sequence of a VHH72 nanobody or a fragment thereof. VHH72 nanobodies specifically bind the spike proteins of the SARS-CoV-2 virus, and variants thereof. As described herein, a VHH72 nanobody comprises SEQ ID NO: 1 or 2.

In certain embodiments, the nanobody comprises an amino acid sequence with at least 80% sequence identity with SEQ ID NO: 2:

(SEQ ID NO: 2)
QVQLQESGGGLVQAGGSLRLSCAASGRTFSEYAMGWFRQAPGKEREFVAT
ISWSGGSTYYTDSVKGRFTISRDNAKNTVYLQMNSLKPDDTAVYYCAAAG
LGTVVSEWDYDYDYWGQGTQVTVSSGS.

In certain embodiments, SEQ ID NO: 2 comprises a V104K amino acid substitution (e.g., and the fluorogenic small molecule is conjugated to the substituted lysine (K) at position 104). In certain embodiments, SEQ ID NO: 2 comprises a V104K amino acid substitution, and all other lysines (Ks) in the amino acid sequence are substituted by a different amino acid (e.g., arginine (R), e.g., in order to remove sites for conjugation other than position 104). For example, a nanobody may comprise SEQ ID NO: 4 below:

(SEQ ID NO: 4)
QVQLQESGGGLVQAGGSLRLSCAASGRTFSEYAMGWFRQAPGREREFVAT
ISWSGGSTYYTDSVRGRFTISRDNARNTVYLQMNSLRPDDTAVYYCAAAG
LGTKVSEWDYDYDYWGQGTQVTVSSGS.

In other aspects, provided herein are fluorogenic sensors based on H11-H4 and sdAb-B6 nanobodies, which are also SARS-CoV-2 binding nanobodies. In yet other aspects, provided herein are fluorogenic sensors based on NbALFA, an ALFA-tag binding nanobody.

In certain embodiments, the target is a small molecule (e.g., an endogenous small molecule such as cortisol) and the fluorogenic sensor can be used to detect said small molecule. In certain embodiments, the fluorogenic sensor comprises a nanobody that binds cortisol (e.g., NbCor). In certain embodiments, the nanobody comprises an amino acid sequence with at least 80% sequence identity with the sequence of NbCor or fragment thereof. NbCor nanobodies specifically bind the small molecule target cortisol.

In another aspect, provided herein are fluorogenic sensors for detecting targets (e.g., antigens) comprising: a mini-protein, and a fluorogenic small molecule conjugated to the mini-protein (e.g., at or around a target-binding domain of the mini-protein, e.g., at or around an antigen-binding domain of the mini-protein). In certain embodiments, the mini-protein specifically binds a pathogen (e.g., a virus), and the fluorogenic sensor can be used to detect said pathogen. In certain embodiments, the mini-protein specifically binds a spike protein of a coronavirus or variant thereof, and the fluorogenic sensor can be used to detect said coronavirus or variant thereof. In certain embodiments, the mini-protein specifically binds a spike protein of a SARS-CoV-2 virus or variant thereof, and the fluorogenic sensor can be used to detect a SARS-CoV-2 virus or variant thereof. For example, provided herein in certain embodiments are fluorogenic sensors based on the SARS-CoV-2 binding mini-protein, LCB3.

In certain embodiments, the fluorogenic small molecule is conjugated to an amino acid at or around a target-binding domain (e.g., antigen-binding domain) of the protein (e.g., nanobody or mini-protein). In certain embodiments, the fluorogenic small molecule is conjugated to an amino acid in a target-binding domain (e.g., antigen-binding domain) of the protein (e.g., nanobody or mini-protein). In certain embodiments, the fluorogenic small molecule is conjugated to a lysine (K) or cysteine (C) residue at or around a target-binding domain (e.g., antigen-binding domain) of the protein (e.g., nanobody or mini-protein). In certain embodiments, the protein (e.g., nanobody or mini-protein) comprises one or more amino acids substituted by lysine (K) or cysteine (C), wherein the fluorogenic small molecule is conjugated to one of said lysines or cysteines.

Also provided herein are compounds (i.e., “fluorogenic probes”) comprising a fluorogenic small molecule and a reactive moiety. The reactive moieties of the compounds are used to conjugate the compounds to proteins (e.g., nanobodies or mini-proteins) to form the fluorogenic sensors described herein. In certain embodiments, a reactive moiety selectively reacts with amines or with thiols. This selective reactivity can allow for selective conjugation of the fluorogenic probes to lysine (K) or cysteine (C) residues of the proteins (e.g., nanobodies or mini-proteins). This selective functionalization can lead to improved fluorogenic sensors with less off-target labeling and/or background fluorescence. Examples of biochemically selective fluorogenic probes are described herein.

Non-limiting examples of fluorogenic probes which can selectively react with thiols (e.g., cysteine residues) include the following:

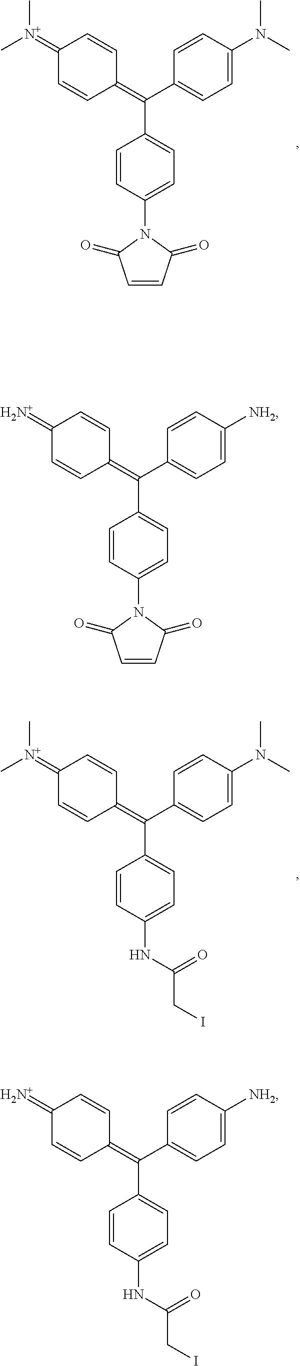

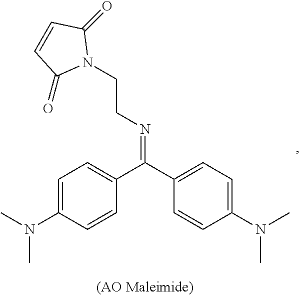

and salts, stereoisomers, and tautomers thereof. Other examples of thiol- and amine-selective fluorogenic probes are described herein. Other non-limiting examples of fluorogenic probes which selectively react with amines and thiols are provided in FIG. 2A and FIG. 2B.

In other embodiments, the protein (e.g., nanobody or mini-protein) comprises an unnatural amino acid comprising a fluorogenic small molecule (i.e., “fluorogenic amino acid” or “FgAA”). In certain embodiments, the FgAA is in a target-binding domain (e.g., antigen binding domain) of the protein. In certain embodiments, at least one amino acid of the amino sequence of the protein (e.g., nanobody or mini-protein) is substituted by a FgAA. For example, a FgAA can be encoded into the amino acid sequence of the protein (e.g., nanobody or mini-protein) or installed via transpeptidation. One or more FgAAs can be incorporated via ribosomal synthesis of the desired protein (e.g., nanobody or mini-protein). FgAAs can also be incorporated during chemical synthesis of the protein (e.g., nanobody). Examples of fluorogenic amino acids which are considered part of the present disclosure can be found in, e.g., International PCT Application Publication WO 2021/118727, published Jun. 17, 2021, the entire contents of which is incorporated herein by reference.

In another aspect, provided herein are methods of determining the presence of a target (e.g., antigen) in a sample, the methods comprising: (i) contacting a sample (e.g., biological sample) with a fluorogenic sensor provided herein; and (ii.a) measuring or observing the fluorescence of the sample or (ii.b) measuring or observing a fluorescence lifetime change of the sample. As described herein, the fluorescence of the sample may increase/decrease upon binding of the fluorogenic sensor to the target (e.g., antigen). Also provided herein are methods of detecting a target (e.g., antigen), the methods comprising: (i) contacting the target (e.g., antigen) with a fluorogenic sensor provided herein; and (ii.a) measuring or observing the fluorescence of the fluorogenic sensor or (ii.b) measuring or observing a fluorescence lifetime change of the fluorogenic sensor. In certain embodiments, the target is an antigen. In certain embodiments, the antigen is a pathogen (e.g., virus, e.g., SARS-CoV-2 or a variant thereof). In certain embodiments, the target is ALFA-tag (e.g., a bacterial cell expressing ALFA-tagged proteins). In certain embodiments, the target is a small molecule such as cortisol.

Also provided herein are kits comprising a fluorogenic sensor provided herein. In certain embodiments, the kit is useful for detecting a target (e.g., an antigen). In certain embodiments, the kit is useful for detecting a pathogen (e.g., virus, e.g., SARS-CoV-2 or a variant thereof) according to a method described herein. Optionally, a kit provided herein will include instructions for use.

The details of certain embodiments of the disclosure are set forth in the Detailed Description of Certain Embodiments, as described below. Other features, objects, and advantages of the disclosure will be apparent from the Definitions, Examples, Figures, and Claims.

### Definitions

### General Definitions

The following definitions are general terms used throughout the present application.

The term “fluorogenic sensor” refers to a target-binding molecule (e.g., a protein, e.g., a nanobody or mini-protein) comprising a fluorogenic small molecule, that can be used to detect binding of the target-binding molecule to the target (e.g., to detect the presence of said target). The target-binding molecule may specifically bind the target. Upon binding of the target-binding molecule to the target, the fluorescence of the fluorogenic small molecule may increase or decrease, thereby “sensing” the target. I additon or alternatively, he fluorescence lifetime of the fluorogenic sensor may detectably change. In other words, an increase/decrease in fluorescence of the fluorogenic sensor or change in fluorescence lifetime of the sensor is indicative of binding of the target-binding molecule to the target, and therefore indicative of the presence of the target. In certain embodiments, the target is an antigen.

The term “target” or “target molecule” are used interchangeably, and as used herein refer any molecule or molecular structure (e.g., protein, antigen, small molecule) which is capable of being bound by a protein. As described herein, in certain embodiments, the target is an antigen, which is capable of being bound by an antigen-binding molecule (e.g., antibody, nanobody, mini-protein). In certain embodiments, the target is a small molecule (e.g., an endogenous small molecule such as cortisol).

The term “antigen” is a molecule or molecular structure, such as may be present on the outside of a pathogen (e.g., virus), that can be bound by an antigen-specific protein (e.g., antibody or nanobody). Antigens most often comprise proteins, peptides, and polysaccharides. The presence of antigens in the body normally triggers an immune response and are thereafter targeted for binding by antibodies. Examples of antigens include viruses, e.g., spike proteins of coronaviruses and variants thereof, e.g., spike proteins of the SARS-CoV-2 virus and variants thereof. In certain embodiments, the antigen is a small molecule.

The terms “protein,” “peptide,” and “polypeptide” are used interchangeably and refer to a polymer of amino acid residues linked together by peptide bonds. The terms refer to peptides, polypeptides, and proteins, of any size, structure, or function. Typically, a protein will be at least three amino acids long, or at least the length required by an amino acid sequence provided herein. A protein may refer to an individual peptide or a collection of proteins. Proteins provided herein can include natural amino acids and/or unnatural amino acids (i.e., compounds that do not occur in nature but that can be incorporated into a peptide chain) in any combination. A protein may be a fragment or modified version of a naturally occurring protein. A protein may be naturally occurring, recombinant, synthetic, or any combination of these.

The term “nanobody” (Nanobody®) refers to a single-domain antibody (“sdAb”). A single-domain antibody is an antibody fragment consisting of a single monomeric variable antibody domain. Like full antibodies, single-domain antibodies are able to bind selectively to specific antigens. In certain embodiments, a nanobody will have a molecular weight of 12-15 kDa, inclusive.

A “target-binding domain” of a protein (e.g., nanobody) is a segment of the protein responsible for binding a target molecule. For example, an “antigen-binding domain” of a protein (e.g., nanobody) is a segment of the protein responsible for binding an antigen. A binding domain may be a group of consecutive amino acids of the amino sequence of the protein. In some instances, a protein (e.g., nanobody) provided herein will comprise more than one (e.g., 1, 2, 3) different binding domains.

The term “amino acid” refers to a molecule containing both an amino group and a carboxyl group. Amino acids include alpha-amino acids and beta-amino acids, the structures of which are depicted below. In certain embodiments, an amino acid is an alpha-amino acid. Each amino acid referred to herein may be denoted by a 1- to 4-letter code as commonly accepted in the art and/or as indicated below.

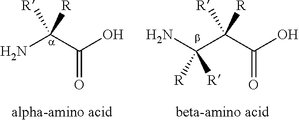

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), unnatural alpha-amino acids, natural beta-amino acids (e.g., beta-alanine), and unnatural beta-amino acids.

Exemplary natural alpha-amino acids (with one-letter code provided in parentheses) include L-alanine (A), L-arginine (R), L-asparagine (N), L-aspartic acid (D), L-cysteine (C), L-glutamic acid (E), L-glutamine (Q), glycine (G), L-histidine (H), L-isoleucine (I), L-leucine (L), L-lysine (K), L-methionine (M), L-phenylalanine (F), L-proline (P), L-serine (S), L-threonine (T), L-tryptophan (W), L-tyrosine (Y), and L-valine (V).

Exemplary unnatural alpha-amino acids include D-arginine, D-asparagine, D-aspartic acid, D-cysteine, D-glutamic acid, D-glutamine, D-histidine, D-isoleucine, D-leucine, D-lysine, D-methionine, D-phenylalanine, D-proline, D-serine, D-threonine, D-tryptophan, D-tyrosine, D-valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α-methyl-methionine, α-methyl-phenylalanine, α-methyl-proline, α-methyl-serine, α-methyl-threonine, α-methyl-tryptophan, α-methyl-tyrosine, α-methyl-valine, norleucine, and terminally unsaturated alpha-amino acids. There are many known unnatural amino acids any of which may be included in the peptides of the present disclosure. See for example, S. Hunt, The Non-Protein Amino Acids: In Chemistry and Biochemistry of the Amino Acids, edited by G. C. Barrett, Chapman and Hall, 1985. Unnatural amino acids also include amino acids comprising nitrogen substituents.

The term “amino acid substitution” when used in reference to an amino acid sequence refers to an amino acid of the amino acid sequence being replaced by a different amino acid (e.g., replaced by a natural or unnatural amino acid). An amino acid sequence provided herein may comprise or include one or more amino acid substitutions. Specific amino acid substitutions are denoted by commonly used colloquial nomenclature in the art of peptide sequencing to denote amino acid sequence variations. For example, when referring to SEQ ID NO: 1 or 2, the amino acid substitution “V104K” refers to replacing the valine (V) at position 104 of the amino acid sequence with lysine (K), resulting in a new amino acid sequence. In certain embodiments, an amino acid sequence provided herein can comprise 0, 1, 2, 3, 4, 5, 6, 7, 8, 9, 10, 11, 12, 13, 14, 15, 16, 17, 18, 19, 20, 21, 22, 23, 24, or 25 amino acid substitutions. In certain embodiments, an amino acid of an amino acid sequence provided herein is substituted by a fluorogenic amino acid (FgAA).

The term “amino acid addition” when used in reference to an amino acid sequence refers to an amino acid (e.g., a natural or unnatural amino acid) being inserted between two amino acids of the amino acid sequence. Standard colloquial nomenclature is used to represent specific amino additions (e.g., when referring to SEQ ID NO: 1 or 2, “V104_V105insX” denotes that a hypothetical amino acid X is inserted between amino acids V104 and V105 of the amino acid sequence, resulting in a new amino acid sequence). In certain embodiments, an amino acid sequence herein can comprise 0, 1, 2, 3, 4, 5, 6, 7, 8, 9, 10, 11, 12, 13, 14, 15, 16, 17, 18, 19, 20, 21, 22, 23, 24, or 25 amino acid additions.

The term “amino acid deletion” when used in reference to an amino acid sequence refers to an amino acid of the amino acid sequence being deleted from the amino acid sequence. Standard colloquial nomenclature is used to represent specific amino deletions (e.g., when referring to SEQ ID NO: 1 or 2, “V104del” denotes that the amino acid V104 is deleted from the sequence, resulting in a new amino acid sequence). In certain embodiments, an amino acid sequence herein can comprise 0, 1, 2, 3, 4, 5, 6, 7, 8, 9, 10, 11, 12, 13, 14, 15, 16, 17, 18, 19, 20, 21, 22, 23, 24, or 25 amino acid deletions.

For purposes of the present disclosure, a fluorogenic sensor denoted by a specific amino acid substitution (e.g., “V104K”), implies that the fluorogenic probe is conjugated to the amino acid at that position unless otherwise specified.

The term “fluorogenic small molecule” or “fluorophore” refers to a small molecule capable of emitting absorbed light, i.e., fluorescing. In certain embodiments, a fluorogenic small molecule can increase/decrease in fluorescence (i.e., “turn on”) in response to changes in viscosity, polarity, or other physical changes. In some embodiments, the fluorogenic small molecule exhibits a detectable change in fluorescence lifetime.

“Fluorescence” is the visible or invisible emission of light by a substance that has absorbed light or other electromagnetic radiation. It can be measured, e.g., by fluorescence microscopy. In certain embodiments, fluorescence is visible and can be detected by the naked eye. In certain embodiments, the detection is colorimetric.

Fluorophores such as the fluorogenic sensors provided herein have distinct fluorescence lifetime signatures, which can be detected, e.g., by a fluorescence lifetime microscopy. “Fluorescence lifetime” (FLT) is the time a fluorophore spends in the excited state before emitting a photon and returning to the ground state. Similar to fluorescence intensity, fluorogenic sensors also significantly change their fluorescence lifetimes based on the micro environment they are in. For example, when a viscosity sensor is free in solution and unconstrained, the sensor will be “darker” and typically will have a shorter fluorescence lifetime. On the other hand, when the sensor is physically restricted (e.g., in higher viscosity environments), they become brighter and show a signature, longer fluorescence lifetime.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

As used herein, the term “conjugated” when used with respect to two or more molecules, means that the molecules are physically associated or connected with one another, either directly (i.e., via a covalent bond) or via one or more additional moieties that serves as a linking agent (i.e., “linker”), to form a structure that is sufficiently stable so that the moieties remain physically associated under the conditions in which the structure is used, e.g., physiological conditions. For example, a fluorogenic small molecule provided herein can be “conjugated” to a protein by reacting a reactive moiety on the fluorogenic small molecule with an amino acid residue (e.g., lysine of cysteine residue) on the protein, thereby forming a covalent linkage between the protein amino acid and the fluorogenic small molecule. In certain embodiments, a fluorogenic small molecule is “conjugated” to a protein when a fluorogenic amino acid (FgAA) (i.e., an amino acid comprising a fluorogenic small molecule) is incorporated into the amino acid sequence of the protein.

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “tautomers” or “tautomeric” refers to two or more interconvertible compounds resulting from at least one migration of a hydrogen atom or electron lone pair, and at least one change in valency (e.g., a single bond to a double bond or vice versa). The exact ratio of the tautomers depends on several factors, including temperature, solvent, and pH. Exemplary tautomerizations include keto-to-enol, amide-to-imide, lactam-to-lactim, enamine-to-imine, and enamine-to-(a different enamine) tautomerizations. Compounds described herein are provided in any and all tautomeric forms. Example of tautomers resulting from the delocalization of electrons (e.g., resonance structures) are shown below:

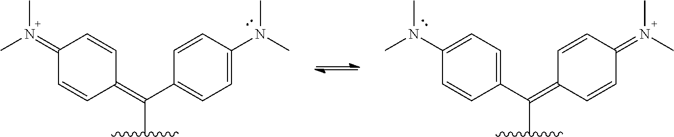

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples may be derived from a nasal swab (e.g., nasopharyngeal swab) such as in the case of a SARS-CoV-2 or influenza test.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses peptides as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₆, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

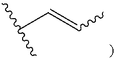

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. Any chemical formula provided herein may also be optionally substituted. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The disclosure is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}—, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R′)₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, Figures, and Claims. The disclosure is not limited in any manner by the above exemplary listing of substituents.

### BRIEF DESCRIPTION OF THE DRAWINGS

The accompanying drawings, which are incorporated in and constitute a part of this specification, illustrate several embodiments of the disclosure and together with the description, provide non-limiting examples of the disclosure.

FIG. 1. Illustration of fluorogenic sensors provided herein. Fluorogenic probes or fluorogenic amino acids are incorporated into a protein. Upon binding of the protein to a target (e.g., antigen), the sensors fluoresce (i.e., “turn on”), indicating the presence of the target.

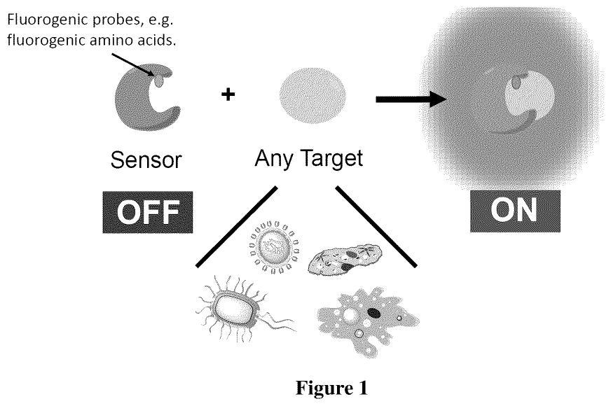

FIG. 2A. Examples of thiol-selective (e.g., cysteine-selective) fluorogenic probes.

FIG. 2B. Examples of amine-selective (e.g., lysine-selective) fluorogenic probes.

FIG. 3. Different VHH72 variants conjugated to AO-maleimide (“AO-Mal”) and their fluorescence response in the presence of SARS-CoV-2 spike protein. Each graph indicates an amino acid substitution to install a cysteine (C) residue into a VHH72 nanobody, and in each case AO-Mal was conjugated to the cysteine at that position. As shown, VHH72 nanobody with V104C mutation (with AO-Mal conjugated at this position) showed the greatest increase in fluorescence upon binding to SARS-CoV-2 spike protein. Each nanobody includes SEQ ID NO: 2 with the captioned amino acid substitution, a histidine tag and thrombin cleavage sequence (MGSSHHHHHHSSGLVPRGSH (SEQ ID NO: 7)) at the N-terminus, and an mRNA display tag (GGSGGGSGGGSG (SEQ ID NO: 8)) at the C-terminus. The structure of AO-maleimide is shown below:

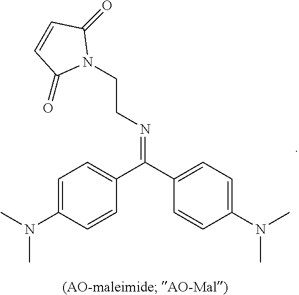

FIG. 4. Fluorogenic sensors comprising different fluorogenic probes (BADAN, IANBD, IAEDANS) conjugated to VHH72 nanobody variant W108C and their fluorescence response in the presence of SARS-CoV-2 spike protein. Fluorogenic probes are conjugated at the W108C cysteine. The nanobody includes SEQ ID NO: 2 with the amino acid substitution W108C, a histidine tag and thrombin cleavage sequence (MGSSHHHHHHSSGLVPRGSH (SEQ ID NO: 7)) at the N-terminus, and mRNA display tag (GGSGGGSGGGSG (SEQ ID NO: 8)) at the C-terminus.

FIG. 5. Fluorogenic sensors comprising NBDx-NHS conjugated to different VHH72 noK variants. Best response to SARS-CoV-2 spike protein (at 600 ug/mL) was for variants V104K & W108K (both noK variants). Each nanobody includes SEQ ID NO: 2 with the captioned amino acid substitution and all other lysines (K) substituted by arginine (R), a histidine tag and thrombin cleavage sequence (MGSSHHHHHHSSGLVPRGSH (SEQ ID NO: 7)) at the N-terminus, and mRNA display tag (GGSGGGSGGGSG (SEQ ID NO: 8)) at the C-terminus. The fluorogenic probes are conjugated to the sole lysine (K) of the nanobodies.

FIG. 6. Additional screening of different VHH72 noK variants conjugated to NBDx-NHS. Each nanobody includes SEQ ID NO: 2 with the captioned amino acid substitution and all other lysines (K) substituted by arginine (R), a histidine tag and thrombin cleavage sequence (MGSSHHHHHHSSGLVPRGSH (SEQ ID NO: 7)) at the N-terminus, and mRNA display tag (GGSGGGSGGGSG (SEQ ID NO: 8)) at the C-terminus. The fluorogenic probes are conjugated to the sole lysine (K) of the nanobodies.

FIG. 7. VHH72 noK V104K (SEQ ID NO: 6) conjugated to NBD (NBDx-NHS) tested at different SARS-CoV-2 spike protein concentrations. NBDx-NHS is conjugated to the sole lysine (K) of the nanobody. Sensitivity limit seems to be ˜50 nM spike protein, which is estimated to be within the amount of spike protein in patient samples.

FIG. 8. Sensors comprising different fluorogenic probes conjugated to VHH72 W108C and their fluorescence response in the presence of SARS-CoV-2 spike protein. Fluorogenic probes are conjugated at the W108C cysteine. The nanobody includes SEQ ID NO: 2 with the amino acid substitution W108C, a histidine tag and thrombin cleavage sequence (MGSSHHHHHHSSGLVPRGSH (SEQ ID NO: 7)) at the N-terminus, and mRNA display tag (GGSGGGSGGGSG (SEQ ID NO: 8)) at the C-terminus.

FIG. 9. Outline of a FRET labeling scheme using VHH72 noK V104K (SEQ ID NO: 6) that employs thrombin cleavage and can increase the detection sensitivity of the sensors. TMR (tetramethylrhodamine) is a fluorescent probe that is not environmentally sensitive. As shown, the emission spectra overlap of NBD-only labeled sensor and TMR-only labeled sensor indicates that they are good FRET partners. In the presence of the SARS-CoV-2 spike protein, the NBD→TMR FRET significantly increases.

FIG. 10. Fluorogenic amino acid (FgAA) Cou was encoded into the interface of a VHH72 nanobody, substituting the Trp 108 (SEQ ID NO: 2) by amber suppression technology (VHH72 W108UAG) in BL21. These purified Cou-containing nanobodies showed fluorescence increase upon binding the Spike protein (˜1.4 fold).

FIG. 11. NanoX (VHH72 noK V104NBDxK) selectively responds to the SARS-CoV-2 spike protein even in complex environments.

FIG. 12. NanoX responds specifically to various SARS-CoV-2 RBD variants of concern by increasing its fluorescence emission up to 100-fold (a significant fluorescence increase that is rarely observed in biosensors) but not to the RBD from MERS-CoV, indicating that the sensor preserves the specificity of VHH72.

FIG. 13. The specific sensitivity of nanoX also allows the wash-free in situ localization microscopy of its target in cells expressing SARS-CoV-2 spike protein.

FIG. 14. The signal enhancement with nanoX is rapid (i.e., instantaneous), within a 500-millisecond window, and ratiometric with a dynamic range of 10⁻⁷ M-10⁻⁵ M and an EC₅₀ of ˜500 nM comparable in both buffer and in human serum.

FIG. 15. Fold fluorescence in the presence of SARS CoV-2 spike protein is also indicated for sensors comprising H11-H4 noK F29K, sdAb-B6 noK K65, and LCB3 H19C. Fold fluorescence in the presence of ALFA-tag is indicated for sensor comprising NbALFA M63C.

FIGS. 16A-16B. Microscopy data was collected using an ALFA-tag biosensor and a Corynebacterium glutamicum bacterium that is expressing an ALFA tagged porin protein complex on its surface. FIG. 16A shows wash-free imaging of ALFA tagged Corynebacterium glutamicum porins with the ALFA sensor. Phase microscopy (left) and fluorescence channel that shows the localization of the porins via the binding-activated ALFA sensor (right). FIG. 16B shows phase microscopy (each frame represents increments of 20 min time-points) (top) and fluorescence channel that shows ALFA-tagged poring localization and relocalization as the cells grow and divide over a period of about four hours (bottom).

FIGS. 17A-17C. Fluorogenic sensors comprising NbCor nanobodies detect cortisol (a small molecule). Different NbCor variants with lysine (FIG. 17B) or cysteine (FIG. 17A) residues on the binding interface were conjugated with various fluorogenic small molecules and tested for signal upon cortisol binding. The fluorogenic sensors in FIG. 17A are as follows (relative to SEQ ID NO: 18): mutc009: V24C+BADAN; mutc010: N27C+Auramine O maleimide; mutc011: T28C+dansyl maleimide; mutc012: G29C+ABD-F; mutc014: W34C+Auramine O maleimide. The fluorogenic sensors in FIG. 17B are as follows (relative to SEQ ID NO: 18): mutc004: V24K, K68R, K79R, K80R, and K90R+cy3 NHS; mutc007: A78K, K68R, K79R, K80R, and K90R+cy3 NHS; mutc008: K68R, K80R, and K90R+BODIPY-Rot-NHS (fluorogenic probe conjugated at K79). The fluorogenic sensors in FIG. 17C are as follows (relative to SEQ ID NO: 18): mutc015: T53C+IANBD; mutc010: N27C+IANBD; mutc011: T28C+IANBD; mutc013: S30C+IANBD. In each instance (except for mutc008) the fluorogenic probe is conjugated to the cysteine or lysine at the substitued positon indicated.

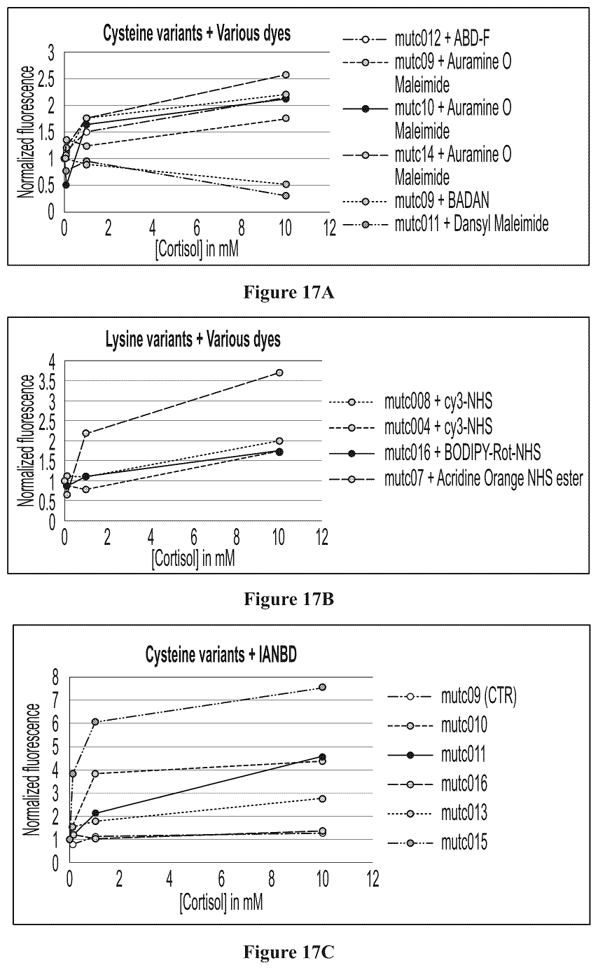

FIGS. 18A and 18B. Fluorogenic sensors provided herein can be used in a new assay format for detecting target molecules. As shown in FIG. 18A, fluorogenic sensors (e.g., ALFA-tag nanobody sensors) can be localized on solid support such as beads (e.g., Ni-NTA beads) and added to a medium containing the target of interest. Any increase/decrease in fluorescence of the beads or change in fluorescence lifetime of the beads can be observed or measured after binding to the target of interest (e.g., ALFA peptides). This enables real-time monitoring of scientific experiments, or industrial processes, or other targets of interest in other complex settings that previously were only detectable using additional lengthy steps. FIG. 18B shows data for the assay described above using an ALFA-tag nanobody sensor localized on Ni-NTA beads. An increase in fluorescence is observed with an increase in ALFA peptide concentration. The dashed line (- - - -) is the background (negative control).

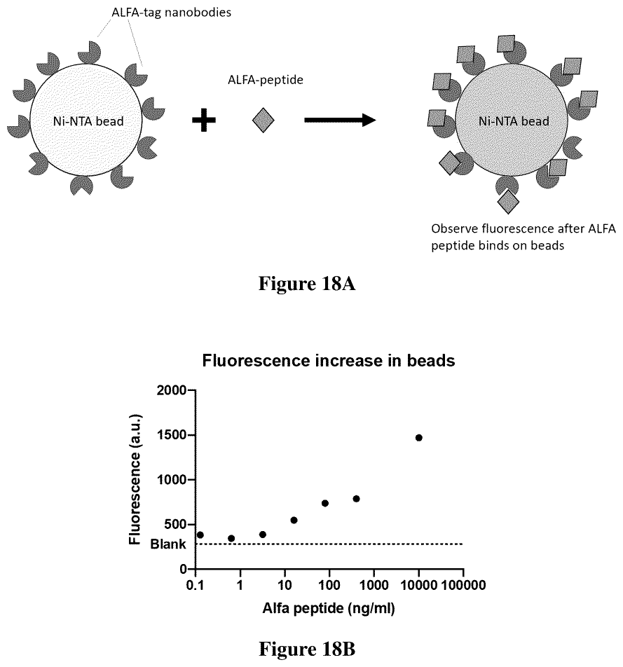

### DETAILED DESCRIPTION OF CERTAIN EMBODIMENTS

Provided herein are fluorogenic sensors which can be used to detect target molecules (e.g., antigens). In general, the fluorogenic sensors provided herein comprise a protein (e.g., antibody, nanobody, mini-protein, designed ankyrin repeat protein (DARPin), monobody) with a fluorogenic small molecule conjugated at or around a target-binding domain of the protein. The protein (e.g., nanobody or mini-protein) may specifically bind said target (e.g., antigen). Upon binding of the protein to said target (e.g., antigen), the fluorogenic small molecule may increase/decrease in fluorescence (i.e., “turn on”) or detectably change its fluorescence lifetime in response to changes in polarity, viscosity, spatial constraints, or other physical changes. This change may be indicative of binding of the protein (e.g., nanobody or mini-protein) to the target (e.g., antigen), and therefore indicative of the presence of the target (e.g., antigens).

Also provided herein are compounds (i.e., fluorogenic probes) used in the fluorogenic sensors provided herein, methods of detecting targets (e.g., antigens) using the fluorogenic sensors, kits comprising the fluorogenic sensors, and other aspects.

### Fluorogenic Sensors for Detecting Targets

Provided herein are fluorogenic sensors for detecting targets comprising: a protein (e.g., a protein that specifically binds the target); and a fluorogenic small molecule conjugated at or around a target-binding domain of the protein. In certain embodiments, the fluorogenic small molecule conjugated to a target-binding domain of the protein. In certain embodiments, the protein is an antibody, nanobody, mini-protein, designed ankyrin repeat protein (DARPin), or monobody. In certain embodiments, the protein specifically binds the target. In certain embodiments, the target is an antigen.

Provided herein are fluorogenic sensors for detecting antigens comprising: a protein (e.g., a protein that specifically binds an antigen); and a fluorogenic small molecule conjugated at or around an antigen-binding domain of the protein. In certain embodiments, the fluorogenic small molecule conjugated to an antigen-binding domain of the protein. In certain embodiments, the protein is an antibody, nanobody, mini-protein, designed ankyrin repeat protein (DARPin), or monobody. In certain embodiments, the protein specifically binds an antigen (e.g., a pathogen, e.g., a spike protein of a coronavirus or variant thereof, e.g., a spike protein of a SARS-CoV-2 virus or variant thereof).

### Nanobodies

In certain embodiments, the protein of the fluorogenic sensor is a nanobody (i.e., a single-domain antibody). Provided herein are fluorogenic sensors for detecting targets comprising: a nanobody; and a fluorogenic small molecule conjugated at or around a target-binding domain of the nanobody. In certain embodiments, the fluorogenic small molecule is conjugated to a target-binding domain of the nanobody. In certain embodiments, the nanobody specifically binds a target. In certain embodiments, the target is an antigen. Provided herein are fluorogenic sensors for detecting antigens comprising: a nanobody; and a fluorogenic small molecule conjugated at or around an antigen-binding domain of the nanobody. In certain embodiments, the fluorogenic small molecule is conjugated to an antigen-binding domain of the nanobody. In certain embodiments, the nanobody specifically binds an antigen.

In certain embodiments, the nanobody binds a pathogen (e.g., specifically binds a pathogen). In certain embodiments, the pathogen is a virus. In certain embodiments, the pathogen is a coronavirus or variant thereof. In certain embodiments, the pathogen is a SARS-CoV-2 virus or variant thereof. In certain embodiments, the pathogen is an influenza virus or variant thereof (e.g., influenza A, B, C, or D, or a variant thereof)

In certain embodiments, the nanobody binds (e.g., specifically binds) a spike protein of a coronavirus or variant thereof. In certain embodiments, the nanobody binds (e.g., specifically binds) a spike protein of a SARS-CoV-2 virus or variant thereof. In certain embodiments, the nanobody binds (e.g., specifically binds) a spike protein of an influenza virus or variant thereof (e.g., influenza A, B, C, or D, or a variant thereof).

In certain embodiments, the nanobody binds (e.g., specifically binds) a nucleocapsid protein of a coronavirus or variant thereof. In certain embodiments, the nanobody binds (e.g., specifically binds) a nucleocapsid protein of a SARS-CoV-2 virus or variant thereof. In certain embodiments, the nanobody binds (e.g., specifically binds) a nucleocapsid protein of an influenza virus or variant thereof (e.g., influenza A, B, C, or D, or a variant thereof).

In certain embodiments, the nanobody binds (e.g., specifically binds) an ALFA-tag protein. In certain embodiments, the nanobody binds (e.g., specifically binds) an ALFA-tag protein on a bacterial cell (e.g., an ALFA-tag protein expressed on a bacterial cell).

In certain embodiments, the nanobody binds (e.g., specifically binds) a small molecule (e.g., an endogenous small molecule). In certain embodiments, the nanobody binds (e.g., specifically binds) cortisol.

### VHH72 Nanobodies

VHH72 nanobodies specifically bind spike proteins of the SARS-CoV-2 virus and variants thereof. In certain embodiments, the nanobody comprises a VHH72 nanobody or a fragment thereof. In certain embodiments, the nanobody comprises an amino acid sequence with at least 80% sequence identity with the sequence of VHH72 nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a VHH72 nanobody, or a fragment thereof. VHH72 nanobodies comprise SEQ ID NO: 1 or 2.

In certain embodiments, the nanobody comprises an amino acid sequence with at least 80% sequence identity with SEQ ID NO: 1:

(SEQ ID NO: 1)
QVQLQESGGGLVQAGGSLRLSCAASGRTFSEYAMGWFRQAPGKEREFVAT
ISWSGGSTYYTDSVKGRFTISRDNAKNTVYLQMNSLKPDDTAVYYCAAAG
LGTVVSEWDYDYDYWGQGTQVTVSS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 1. In certain embodiments, the nanobody comprises an amino acid sequence with about 90%, 91%, 92%, 93%, 94%, 95%, 96%, 97%, 98%, 99%, or 100% sequence identity with SEQ ID NO: 1. In certain embodiments, the nanobody comprises an amino acid sequence with 100% sequence identity with SEQ ID NO: 1.

In certain embodiments, the nanobody comprises an amino acid sequence with at least 80% sequence identity with SEQ ID NO: 2:

(SEQ ID NO: 2)
QVQLQESGGGLVQAGGSLRLSCAASGRTFSEYAMGWFRQAPGKEREFVAT
ISWSGGSTYYTDSVKGRFTISRDNAKNTVYLQMNSLKPDDTAVYYCAAAG
LGTVVSEWDYDYDYWGQGTQVTVSSGS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 2. In certain embodiments, the nanobody comprises an amino acid sequence with about 90%, 91%, 92%, 93%, 94%, 95%, 96%, 97%, 98%, 99%, or 100% sequence identity with SEQ ID NO: 2. In certain embodiments, the nanobody comprises an amino acid sequence with 100% sequence identity with SEQ ID NO: 2.

As described herein, the fluorogenic sensors comprise a fluorogenic small molecule conjugated to an antigen-binding domain of the nanobody. In certain embodiments, an antigen-binding domain is from amino acids 26-35 of SEQ ID NO: 1 or 2, or a variant thereof, amino acids 50-59 of SEQ ID NO: 1 or 2, or a variant thereof, or amino acids 99-114 of SEQ ID NO: 1 or 2, or a variant thereof. In certain embodiments, an antigen-binding domain is from amino acids G26-G35 of SEQ ID NO: 1 or 2, or a variant thereof, amino acids T50-Y59 of SEQ ID NO: 1 or 2, or a variant thereof, or amino acids A99-Y114 of SEQ ID NO: 1 or 2, or a variant thereof. Possible antigen-binding domains are denoted by the bolded and underlined sequences of amino acids in SEQ ID NO: 1 and 2 below:

(SEQ ID NO: 1)
QVQLQESGGGLVQAGGSLRLSCAASGRTFSEYAMGWFRQAPGKEREFVAT
ISWSGGSTYYTDSVKGRFTISRDNAKNTVYLQMNSLKPDDTAVYYCAAAG
LGTVVSEWDYDYDYWGQGTQVTVSS,
(SEQ ID NO: 2)
QVQLQESGGGLVQAGGSLRLSCAASGRTFSEYAMGWFRQAPGKEREFVAT
ISWSGGSTYYTDSVKGRFTISRDNAKNTVYLQMNSLKPDDTAVYYCAAAG
LGTVVSEWDYDYDYWGQGTQVTVSSGS.

In certain embodiments, the antigen-binding domain is from amino acids 99-114 of SEQ ID NO: 1 or 2 (e.g., A99-Y114) or a variant thereof.

In certain embodiments, the fluorogenic small molecule is conjugated to an amino acid at position 51, 53, 56, 59, 102, 103, 104, 105, 108, 110, or 115 of SEQ ID NO: 1 or 2, or a variant thereof. In certain embodiments, the fluorogenic small molecule is conjugated to an amino acid at position 51 of SEQ ID NO: 1 or 2, or a variant thereof. In certain embodiments, the fluorogenic small molecule is conjugated to an amino acid at position 53 of SEQ ID NO: 1 or 2, or a variant thereof. In certain embodiments, the fluorogenic small molecule is conjugated to an amino acid at position 56 of SEQ ID NO: 1 or 2, or a variant thereof. In certain embodiments, the fluorogenic small molecule is conjugated to an amino acid at position 59 of SEQ ID NO: 1 or 2, or a variant thereof. In certain embodiments, the fluorogenic small molecule is conjugated to an amino acid at position 102 of SEQ ID NO: 1 or 2, or a variant thereof. In certain embodiments, the fluorogenic small molecule is conjugated to an amino acid at position 103 of SEQ ID NO: 1 or 2, or a variant thereof. In certain embodiments, the fluorogenic small molecule is conjugated to an amino acid at position 104 of SEQ ID NO: 1 or 2, or a variant thereof. In certain embodiments, the fluorogenic small molecule is conjugated to an amino acid at position 105 of SEQ ID NO: 1 or 2, or a variant thereof. In certain embodiments, the fluorogenic small molecule is conjugated to an amino acid at position 108 of SEQ ID NO: 1 or 2, or a variant thereof. In certain embodiments, the fluorogenic small molecule is conjugated to an amino acid at position 110 of SEQ ID NO: 1 or 2, or a variant thereof. In certain embodiments, the fluorogenic small molecule is conjugated to an amino acid at position 115 of SEQ ID NO: 1 or 2, or a variant thereof.

In certain embodiments, the fluorogenic small molecule is conjugated to an amine (—NH₂)-containing residue of the protein (e.g., nanobody). In certain embodiments, the fluorogenic small molecule is conjugated to a lysine (K) residue of the protein. In certain embodiments, the protein comprises one or more amino acids substituted by lysine (K) and, the fluorogenic small molecule is conjugated to one of said lysine.

For example, in certain embodiments, the nanobody comprises SEQ ID NO: 1 or 2 with at least one amino acid substitution selected from W53K, V104K, V105K, W108K, Y110K, and W115K; and wherein the fluorogenic small molecule is conjugated to the lysine at that position. In certain embodiments, the nanobody comprises SEQ ID NO: 1 or 2, with a W53K amino acid substitution; and wherein the fluorogenic small molecule is conjugated to the lysine at that position. In certain embodiments, the nanobody comprises SEQ ID NO: 1 or 2, with a V104K amino acid substitution; and wherein the fluorogenic small molecule is conjugated to the lysine at that position. In certain embodiments, the nanobody comprises SEQ ID NO: 1 or 2, with a V105K amino acid substitution; and wherein the fluorogenic small molecule is conjugated to the lysine at that position. In certain embodiments, the nanobody comprises SEQ ID NO: 1 or 2, with a W108K amino acid substitution; and wherein the fluorogenic small molecule is conjugated to the lysine at that position. In certain embodiments, the nanobody comprises SEQ ID NO: 1 or 2, with a Y110K amino acid substitution; and wherein the fluorogenic small molecule is conjugated to the lysine at that position. In certain embodiments, the nanobody comprises SEQ ID NO: 1 or 2, with a W115K amino acid substitution; and wherein the fluorogenic small molecule is conjugated to the lysine at that position. The nanobody may include one or more additional amino acid substitutions (e.g., provided that the nanobody has at least 80% sequence identity with SEQ ID NO: 1 or 2).

In certain embodiments, the nanobody comprises one of the foregoing amino acid substitutions, further wherein one or more other lysines of the nanobody (i.e., other than the lysine resulting from the amino acid substitution) are independently substituted by a different amino acid (i.e., an amino acid other than lysine). In certain embodiments, one or more other lysines of the nanobody are independently substituted by arginine (R). In certain embodiments, all other lysines of the nanobody are independently substituted by a different amino acid (i.e., an amino acid other than lysine). In certain embodiments, all other lysines of the nanobody are independently substituted by arginine (R). These nanobodies may be referred to as “no lysine” or “noK” nanobodies. Limiting the number of lysines in the nanobody can lead to greater site selectivity for conjugation of the fluorogenic probe when lysine-selective fluorogenic probes are used. This is turn can lead to reduced background fluorescence for the fluorogenic sensors.

In certain embodiments, one or more lysines at positions 43, 65, 76, and/or 87 of SEQ ID NO: 1 or 2 are independently substituted by a different amino acid. In certain embodiments, one or more lysines at positions 43, 65, 76, and/or 87 of SEQ ID NO: 1 or 2 are independently substituted by arginine (R). In certain embodiments, SEQ ID NO: 1 or 2 further comprises one or more amino acid substitutions selected from K43R, K65R, K76R, and K87R.

For example, a nanobody may comprise SEQ ID NO: 2 with a V104K amino acid substitution, further wherein all other lysines of the amino acid sequence are substituted by arginine. For example, a nanobody may comprise SEQ ID NO: 3 below:

(SEQ ID NO: 3)
QVQLQESGGGLVQAGGSLRLSCAASGRTFSEYAMGWFRQAPGREREFVAT
ISWSGGSTYYTDSVRGRFTISRDNARNTVYLQMNSLRPDDTAVYYCAAAG
LGTKVSEWDYDYDYWGQGTQVTVSSGS.

In certain embodiments, the nanobody comprises an amino acid sequence with at least 80% sequence identity with SEQ ID NO: 3. In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 3. In certain embodiments, the nanobody comprises an amino acid sequence with about 90%, 91%, 92%, 93%, 94%, 95%, 96%, 97%, 98%, 99%, or 100% sequence identity with SEQ ID NO: 3. In certain embodiments, the nanobody comprises an amino acid sequence with 100% sequence identity with SEQ ID NO: 3.

For example, a nanobody may comprise SEQ ID NO: 2 with a V104K amino acid substitution, further wherein all other lysines of the amino acid sequence are substituted by arginine (“V104K noK”). For example, a nanobody may comprise SEQ ID NO: 4 below (VHH72 noK V104K):

(SEQ ID NO: 4)
QVQLQESGGGLVQAGGSLRLSCAASGRTFSEYAMGWFRQAPGREREFVAT
ISWSGGSTYYTDSVRGRFTISRDNARNTVYLQMNSLRPDDTAVYYCAAAG
LGTKVSEWDYDYDYWGQGTQVTVSSGS.

In certain embodiments, the nanobody comprises an amino acid sequence with at least 80% sequence identity with SEQ ID NO: 4. In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 4. In certain embodiments, the nanobody comprises an amino acid sequence with about 90%, 91%, 92%, 93%, 94%, 95%, 96%, 97%, 98%, 99%, or 100% sequence identity with SEQ ID NO: 4. In certain embodiments, the nanobody comprises an amino acid sequence with 100% sequence identity with SEQ ID NO: 4.

In certain embodiments, the fluorogenic small molecule is conjugated to a thiol (—SH)-containing residue of the nanobody. In certain embodiments, the fluorogenic small molecule is conjugated to a cysteine (C) residue of the nanobody. In certain embodiments, the nanobody comprises one or more amino acids substituted by cysteine (C) and, wherein the fluorogenic small molecule is conjugated to one of said cysteine.

For example, in certain embodiments, the nanobody comprises SEQ ID NO: 1 or 2 with at least one amino acid substitution selected from I51C, W53C, G56C, Y59C, G102C, T103C, V104C, V105C, W108C, Y110C, and W115C; and wherein the fluorogenic small molecule is conjugated to the cysteine at that position. In certain embodiments, the nanobody comprises SEQ ID NO: 1 or 2 with a I51C amino acid substitution; and wherein the fluorogenic small molecule is conjugated to the cysteine at that position. In certain embodiments, the nanobody comprises SEQ ID NO: 1 or 2 with a W53C amino acid substitution; and wherein the fluorogenic small molecule is conjugated to the cysteine at that position. In certain embodiments, the nanobody comprises SEQ ID NO: 1 or 2 with a G56C amino acid substitution; and wherein the fluorogenic small molecule is conjugated to the cysteine at that position. In certain embodiments, the nanobody comprises SEQ ID NO: 1 or 2 with a Y59C amino acid substitution; and wherein the fluorogenic small molecule is conjugated to the cysteine at that position. In certain embodiments, the nanobody comprises SEQ ID NO: 1 or 2 with a G102C amino acid substitution; and wherein the fluorogenic small molecule is conjugated to the cysteine at that position. In certain embodiments, the nanobody comprises SEQ ID NO: 1 or 2 with a T103C amino acid substitution; and wherein the fluorogenic small molecule is conjugated to the cysteine at that position. In certain embodiments, the nanobody comprises SEQ ID NO: 1 or 2 with a V104C amino acid substitution; and wherein the fluorogenic small molecule is conjugated to the cysteine at that position. In certain embodiments, the nanobody comprises SEQ ID NO: 1 or 2 with a V105C amino acid substitution; and wherein the fluorogenic small molecule is conjugated to the cysteine at that position. In certain embodiments, the nanobody comprises SEQ ID NO: 1 or 2 with a W108C amino acid substitution; and wherein the fluorogenic small molecule is conjugated to the cysteine at that position. In certain embodiments, the nanobody comprises SEQ ID NO: 1 or 2 with a Y110C amino acid substitution; and wherein the fluorogenic small molecule is conjugated to the cysteine at that position. In certain embodiments, the nanobody comprises SEQ ID NO: 1 or 2 with a W115C amino acid substitution; and wherein the fluorogenic small molecule is conjugated to the cysteine at that position. The nanobody may include one or more additional amino acid substitutions (e.g., provided that the nanobody has at least 80% sequence identity with SEQ ID NO: 1 or 2).

In certain embodiments, the nanobody comprises one of the foregoing amino acid substitutions, further wherein one or more other cysteines of the nanobody (i.e., other than the cysteine resulting from the amino acid substitution) are independently substituted by a different amino acid (i.e., an amino acid other than cysteine). In certain embodiments, one or more other cysteines of the nanobody are independently substituted by alanine (A) or valine (V). In certain embodiments, all other cysteines of the nanobody are independently substituted by a different amino acid (i.e., an amino acid other than cysteine). In certain embodiments, all other cysteines of the nanobody are independently substituted by alanine (A) or valine (V). These nanobodies may be referred to as “no cysteine” or “noC” nanobodies. Limiting the number of cysteines in the nanobody can lead to greater site selectivity for conjugation of the fluorogenic probe when cysteine-selective fluorogenic probes are used. This is turn can lead to reduced background fluorescence for the fluorogenic sensors.

In certain embodiments, one or both cysteines at positions 22 and/or 96 of SEQ ID NO: 1 or 2 are independently substituted by a different amino acid. In certain embodiments, one or both cysteines at positions 22 and/or 96 of SEQ ID NO: 1 or 2 are independently substituted by valine (V) or alanine (A). In certain embodiments, SEQ ID NO: 1 or 2 further comprises one or more amino acid substitutions selected from C22A, C22V, C96A, and C96V.

### H11-H4 Nanobodies

H11-H4 nanobodies also bind spike proteins of the SARS-CoV-2 virus and variants thereof. See, e.g., Huo et al., Nature structural & molecular biology, vol. 27, 846-854 (2020). In certain embodiments, the nanobody comprises a H11-H4 nanobody or a fragment thereof. In certain embodiments, the nanobody comprises an amino acid sequence with at least 80% sequence identity with the sequence of a H11-H4 nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a H11-H4 nanobody, or a fragment thereof. H11-H4 nanobodies comprise SEQ ID NO: 10.

In certain embodiments, the nanobody comprises an amino acid sequence with at least 80% sequence identity with SEQ ID NO: 10:

(SEQ ID NO: 10)
QVQLVESGGGLMQAGGSLRLSCAVSGRTESTAAMGWFRQAPGKEREFVAA
IRWSGGSAYYADSVKGRFTISRDKAKNTVYLQMNSLKYEDTAVYYCAQTH
YVSYLLSDYATWPYDYWGQGTQVTVSS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 10. In certain embodiments, the nanobody comprises an amino acid sequence with about 90%, 91%, 92%, 93%, 94%, 95%, 96%, 97%, 98%, 99%, or 100% sequence identity with SEQ ID NO: 10. In certain embodiments, the nanobody comprises an amino acid sequence with 100% sequence identity with SEQ ID NO: 10.

As described herein, the fluorogenic sensors comprise a fluorogenic small molecule conjugated to an antigen-binding domain of the nanobody. In certain embodiments, an antigen-binding domain is from amino acids 25-35 or 50-65 of SEQ ID NO: 10, or a variant thereof. In certain embodiments, an antigen-binding domain is from amino acids 95-120 of SEQ ID NO: 10, or a variant thereof. In certain embodiments, the fluorogenic small molecule is conjugated to an amino acid at position 29 of SEQ ID NO: 10, or a variant thereof.

In certain embodiments, the fluorogenic small molecule is conjugated to an amine (—NH₂)-containing residue of the nanobody. In certain embodiments, the fluorogenic small molecule is conjugated to a lysine (K) residue of the nanobody. In certain embodiments, the nanobody comprises one or more amino acids substituted by lysine (K) and, the fluorogenic small molecule is conjugated to one of said lysine.

For example, in certain embodiments, the nanobody comprises SEQ ID NO: 10 with at least one amino acid substitution, wherein the at least one amino acid substitution is F29K, and wherein the fluorogenic small molecule is conjugated to the lysine at that position. The nanobody may include one or more additional amino acid substitutions (e.g., provided that the nanobody has at least 80% sequence identity with SEQ ID NO: 10).

In certain embodiments, the nanobody comprises one of the foregoing amino acid substitutions, further wherein one or more other lysines of the nanobody (i.e., other than the lysine resulting from the amino acid substitution) are independently substituted by a different amino acid (i.e., an amino acid other than lysine). In certain embodiments, one or more other lysines of the nanobody are independently substituted by arginine (R). In certain embodiments, all other lysines of the nanobody are independently substituted by a different amino acid (i.e., an amino acid other than lysine). In certain embodiments, all other lysines of the nanobody are independently substituted by arginine (R). As described herein, these nanobodies may be referred to as “no lysine” or “noK” nanobodies.

For example, a nanobody may comprise SEQ ID NO: 10 with a F29K amino acid substitution, further wherein all other lysines of the amino acid sequence are substituted by arginine (H11-H4 noK F29K; SEQ ID NO: 11 below):

(SEQ ID NO: 11)
QVQLVESGGGLMQAGGSLRLSCAVSGRTKSTAAMGWFRQAPGREREFVAA
IRWSGGSAYYADSVRGRFTISRDRARNTVYLQMNSLRYEDTAVYYCAQTH
YVSYLLSDYATWPYDYWGQGTQVTVSS.

In certain embodiments, the nanobody comprises an amino acid sequence with at least 80% sequence identity with SEQ ID NO: 11. In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 11. In certain embodiments, the nanobody comprises an amino acid sequence with about 90%, 91%, 92%, 93%, 94%, 95%, 96%, 97%, 98%, 99%, or 100% sequence identity with SEQ ID NO: 11. In certain embodiments, the nanobody comprises an amino acid sequence with 100% sequence identity with SEQ ID NO: 11.

sdAb-B6 Nanobodies

sdAb-B6 nanobodies bind nucleocapsid proteins of the SARS-CoV-2 virus and variants thereof. See, e.g., Ye et al., Front. Immunol., vol. 12, 719037 (2021). In certain embodiments, the nanobody comprises a sdAb-B6 nanobody or a fragment thereof. In certain embodiments, the nanobody comprises an amino acid sequence with at least 80% sequence identity with the sequence of a sdAb-B6 nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a sdAb-B6 nanobody, or a fragment thereof. sdAb-B6 nanobodies comprise SEQ ID NO: 12.

In certain embodiments, the nanobody comprises an amino acid sequence with at least 80% sequence identity with SEQ ID NO: 12:

(SEQ ID NO: 12)
VQLQASGGGLVRPGGSLRLSCAASGFTESSYAMMWVRQAPGKGLEWVSAI
NGGGGSTSYADSVKGRFTISRDNAKNTLYLQMNSLKPEDTAVYYCAKYQA
AVHQEKEDYWGQGTQVTVSS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 12. In certain embodiments, the nanobody comprises an amino acid sequence with about 90%, 91%, 92%, 93%, 94%, 95%, 96%, 97%, 98%, 99%, or 100% sequence identity with SEQ ID NO: 12. In certain embodiments, the nanobody comprises an amino acid sequence with 100% sequence identity with SEQ ID NO: 12.

As described herein, the fluorogenic sensors comprise a fluorogenic small molecule conjugated to an antigen-binding domain of the nanobody. In certain embodiments, an antigen-binding domain is from amino acids 50-65 and 95-120 of SEQ ID NO: 12, or a variant thereof. In certain embodiments, the fluorogenic small molecule is conjugated to an amino acid at position 65 of SEQ ID NO: 12, or a variant thereof.

In certain embodiments, the fluorogenic small molecule is conjugated to an amine (—NH₂)-containing residue of the nanobody. In certain embodiments, the fluorogenic small molecule is conjugated to a lysine (K) residue of the nanobody. In certain embodiments, the nanobody comprises one or more lysine (K) residues and the fluorogenic small molecule is conjugated to one of said lysine.

For example, in certain embodiments, the nanobody comprises SEQ ID NO: 12 with a K65 residue, and wherein the fluorogenic small molecule is conjugated to the lysine at that position. In certain embodiments, one or more other lysines of the nanobody (i.e., other than K65) are independently substituted by a different amino acid (i.e., an amino acid other than lysine). In certain embodiments, one or more other lysines of the nanobody are independently substituted by arginine (R). In certain embodiments, all other lysines of the nanobody are independently substituted by a different amino acid (i.e., an amino acid other than lysine). In certain embodiments, all other lysines of the nanobody are independently substituted by arginine (R). As described herein, these nanobodies may be referred to as “no lysine” or “noK” nanobodies.

For example, a nanobody may comprise SEQ ID NO: 12 with K65, wherein all other lysines of the amino acid sequence are substituted by arginine (sdAb-B6 noK K65; SEQ ID NO: 13 below):

(SEQ ID NO: 13)
QSSGEVQLQASGGGLVRPGGSLRLSCAASGFTFSSYAMMWVRQAPGRGLE
WVSAINGGGGSTSYADSVKGRFTISRDNARNTLYLQMNSLRPEDTAVYYC
ARYQAAVHQEREDYWGQGTQVTVSS.

In certain embodiments, the nanobody comprises an amino acid sequence with at least 80% sequence identity with SEQ ID NO: 13. In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 13. In certain embodiments, the nanobody comprises an amino acid sequence with about 90%, 91%, 92%, 93%, 94%, 95%, 96%, 97%, 98%, 99%, or 100% sequence identity with SEQ ID NO: 13. In certain embodiments, the nanobody comprises an amino acid sequence with 100% sequence identity with SEQ ID NO: 13.

### NbALFA Nanobodies

NbALFA nanobodies bind ALFA-tag proteins and can therefore be used to detect ALFA-tag proteins and cells comprising the same (e.g., bacterial cells expressing ALFA-tagged proteins). See, e.g., Götzke et al., Nature Communications vol. 10, 4403 (2019). In certain embodiments, the nanobody comprises a NbALFA nanobody or a fragment thereof. In certain embodiments, the nanobody comprises an amino acid sequence with at least 80% sequence identity with the sequence of a NbALFA nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a NbALFA nanobody, or a fragment thereof. NbALFA nanobodies comprise SEQ ID NO: 16.

In certain embodiments, the nanobody comprises an amino acid sequence with at least 80% sequence identity with SEQ ID NO: 16:

(SEQ ID NO: 16)
VQLQESGGGLVQPGGSLRLSCTASGVTISALNAMAMGWYRQAPGERRVMV
AAVSERGNAMYRESVQGRFTVTRDFTNKMVSLQMDNLKPEDTAVYYCHVL
EDRVDSFHDYWGQGTQVTVSS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 16. In certain embodiments, the nanobody comprises an amino acid sequence with about 90%, 91%, 92%, 93%, 94%, 95%, 96%, 97%, 98%, 99%, or 100% sequence identity with SEQ ID NO: 16. In certain embodiments, the nanobody comprises an amino acid sequence with 100% sequence identity with SEQ ID NO: 16.

As described herein, the fluorogenic sensors comprise a fluorogenic small molecule conjugated to an antigen-binding domain of the nanobody. In certain embodiments, an antigen-binding domain is from amino acids 50-65 and 95-120 of SEQ ID NO: 16, or a variant thereof. In certain embodiments, the fluorogenic small molecule is conjugated to an amino acid at position 63 of SEQ ID NO: 16, or a variant thereof.

In certain embodiments, the fluorogenic small molecule is conjugated to an amine (—NH₂)-containing residue of the nanobody. In certain embodiments, the fluorogenic small molecule is conjugated to a lysine (K) residue of the nanobody. In certain embodiments, the nanobody comprises one or more lysine (K) residues and the fluorogenic small molecule is conjugated to one of said lysine.

In certain embodiments, the fluorogenic small molecule is conjugated to a thiol (—SH)-containing residue of the nanobody. In certain embodiments, the fluorogenic small molecule is conjugated to a cysteine (C) residue of the nanobody. In certain embodiments, the nanobody comprises one or more amino acids substituted by cysteine (C) and, wherein the fluorogenic small molecule is conjugated to one of said cysteine.

In certain embodiments, one or more other lysines of the nanobody are independently substituted by a different amino acid (i.e., an amino acid other than lysine). In certain embodiments, one or more other lysines of the nanobody are independently substituted by arginine (R). In certain embodiments, all other lysines of the nanobody are independently substituted by a different amino acid (i.e., an amino acid other than lysine). In certain embodiments, all other lysines of the nanobody are independently substituted by arginine (R). As described herein, these nanobodies may be referred to as “no lysine” or “noK” nanobodies.

For example, in certain embodiments, the nanobody comprises SEQ ID NO: 16 with at least one amino acid substitution, wherein the at least one amino acid substitution is M63C; and wherein the fluorogenic small molecule is conjugated to the cysteine at that position.

For example, a nanobody may comprise NbALFA noK M63C; (SEQ ID NO: 17 below):

(SEQ ID NO: 17)
VQLQESGGGLVQPGGSLRLSCTASGVTISALNAMAMGWYRQAPGERRVMV
AAVSERGNACYRESVQGRFTVTRDFTNRMVSLQMDNLRPEDTAVYYCHVL
EDRVDSFHDYWGQGTQVTVSS.

In certain embodiments, the nanobody comprises an amino acid sequence with at least 80% sequence identity with SEQ ID NO: 17. In certain embodiments, the mini-protein comprises an amino acid sequence with at least 50%, 55%, 60%, 65%, 70%, 75%, 80%, 85%, 90%, 95%, 98%, 99%, or 100% sequence identity with SEQ ID NO: 17. In certain embodiments, the mini-protein comprises an amino acid sequence with about 90%, 91%, 92%, 93%, 94%, 95%, 96%, 97%, 98%, 99%, or 100% sequence identity with SEQ ID NO: 17. In certain embodiments, the mini-protein comprises an amino acid sequence with 100% sequence identity with SEQ ID NO: 17.

In certain embodiments, the nanobody comprises one of the foregoing amino acid substitutions, further wherein one or more other cysteines of the nanobody (i.e., other than the cysteine resulting from the amino acid substitution) are independently substituted by a different amino acid (i.e., an amino acid other than cysteine). In certain embodiments, one or more other cysteines of the nanobody are independently substituted by alanine (A) or valine (V). In certain embodiments, all other cysteines of the nanobody are independently substituted by a different amino acid (i.e., an amino acid other than cysteine). In certain embodiments, all other cysteines of the nanobody are independently substituted by alanine (A) or valine (V). These mini-protein may be referred to as “no cysteine” or “noC” nanobody.

### NbCor Nanobodies

NbCor nanobodies specifically bind the small molecule cortisol. In certain embodiments, the nanobody comprises a NbCor nanobody or a fragment thereof. In certain embodiments, the nanobody comprises an amino acid sequence with at least 80% sequence identity with the sequence of NbCor nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a NbCor nanobody, or a fragment thereof. NbCor nanobodies comprise SEQ ID NO: 18.

In certain embodiments, the nanobody comprises an amino acid sequence with at least 80% sequence identity with SEQ ID NO: 18:

(SEQ ID NO: 18)
QVQLQESGGGSVQAGGSLRLSCVVSGNTGSTGYWAWFRQGPGTEREGVAA
TYTAGSGTSMTYYADSVKGRFTISQDNAKKTLYLQMNSLKPEDIGMYRCA
STRFAGRWYRDSEYRAWGQGTQVTVSS.

In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 18. In certain embodiments, the nanobody comprises an amino acid sequence with about 90%, 91%, 92%, 93%, 94%, 95%, 96%, 97%, 98%, 99%, or 100% sequence identity with SEQ ID NO: 18. In certain embodiments, the nanobody comprises an amino acid sequence with 100% sequence identity with SEQ ID NO: 18.

In certain embodiments, the fluorogenic small molecule is conjugated to an amino acid at position 24, 78, or 79 of SEQ ID NO: 18, or a variant thereof. In certain embodiments, the fluorogenic small molecule is conjugated to an amino acid at position 53, 27, 28, 30, 24, 29, or 34 of SEQ ID NO: 18, or a variant thereof. In certain embodiments, the fluorogenic small molecule is conjugated to an amino acid at position 24 of SEQ ID NO: 18, or a variant thereof. In certain embodiments, the fluorogenic small molecule is conjugated to an amino acid at position 78 of SEQ ID NO: 18, or a variant thereof. In certain embodiments, the fluorogenic small molecule is conjugated to an amino acid at position 53 of SEQ ID NO: 18, or a variant thereof. In certain embodiments, the fluorogenic small molecule is conjugated to an amino acid at position 27 of SEQ ID NO: 18, or a variant thereof. In certain embodiments, the fluorogenic small molecule is conjugated to an amino acid at position 28 of SEQ ID NO: 18, or a variant thereof. In certain embodiments, the fluorogenic small molecule is conjugated to an amino acid at position 30 of SEQ ID NO: 18, or a variant thereof. In certain embodiments, the fluorogenic small molecule is conjugated to an amino acid at position 53, 29 of SEQ ID NO: 18, or a variant thereof. In certain embodiments, the fluorogenic small molecule is conjugated to an amino acid at position 34 of SEQ ID NO: 18, or a variant thereof.

In certain embodiments, the fluorogenic small molecule is conjugated to an amine (—NH₂)-containing residue of the protein (e.g., nanobody). In certain embodiments, the fluorogenic small molecule is conjugated to a lysine (K) residue of the protein. In certain embodiments, the protein comprises one or more amino acids substituted by lysine (K) and, the fluorogenic small molecule is conjugated to one of said lysine.

For example, in certain embodiments, the nanobody comprises SEQ ID NO: 18 with at least one amino acid substitution selected from V24K and A78K; and wherein the fluorogenic small molecule is conjugated to the lysine at that position. In certain embodiments, the nanobody comprises SEQ ID NO: 18, with a V24K amino acid substitution; and wherein the fluorogenic small molecule is conjugated to the lysine at that position. In certain embodiments, the nanobody comprises SEQ ID NO: 18, with a A78K amino acid substitution; and wherein the fluorogenic small molecule is conjugated to the lysine at that position. In certain embodiments, the fluorogenic small molecule is conjugated to K79 of SEQ ID NO: 18 or a variant thereof. The nanobody may include one or more additional amino acid substitutions (e.g., provided that the nanobody has at least 80% sequence identity with SEQ ID NO: 18).

In certain embodiments, the nanobody comprises one of the foregoing amino acid substitutions, further wherein one or more other lysines of the nanobody (i.e., other than the lysine resulting from the amino acid substitution) are independently substituted by a different amino acid (i.e., an amino acid other than lysine). In certain embodiments, one or more other lysines of the nanobody are independently substituted by arginine (R). In certain embodiments, all other lysines of the nanobody are independently substituted by a different amino acid (i.e., an amino acid other than lysine). In certain embodiments, all other lysines of the nanobody are independently substituted by arginine (R). These nanobodies may be referred to as “no lysine” or “noK” nanobodies.

In certain embodiments, one or more lysines at positions 68, 79, 80, and/or 90 of SEQ ID NO: 18 are independently substituted by a different amino acid. In certain embodiments, one or more lysines at positions 68, 79, 80, and/or 90 of SEQ ID NO: 18 are independently substituted by arginine (R). In certain embodiments, SEQ ID NO: 18 further comprises one or more amino acid substitutions selected from K68R, K79R, K80R, K90R.

In certain embodiments, the nanobody comprises SEQ ID NO: 18 with the following amino acid substitutions: V24K, K68R, K79R, K80R, and K90R. In certain embodiments, the nanobody comprises SEQ ID NO: 18 with the following amino acid substitutions: A78K, K68R, K79R, K80R, and K90R. In certain embodiments, the nanobody comprises SEQ ID NO: 18 with the following amino acid substitutions: K68R, K80R, and K90R.

In certain embodiments, the fluorogenic small molecule is conjugated to a thiol (—SH)-containing residue of the nanobody. In certain embodiments, the fluorogenic small molecule is conjugated to a cysteine (C) residue of the nanobody. In certain embodiments, the nanobody comprises one or more amino acids substituted by cysteine (C) and, wherein the fluorogenic small molecule is conjugated to one of said cysteine.

For example, in certain embodiments, the nanobody comprises SEQ ID NO: 18 with at least one amino acid substitution selected from T53C, N27C, T28C, S30C, V24C, G29C, and W34C; and wherein the fluorogenic small molecule is conjugated to the cysteine at that position. In certain embodiments, the nanobody comprises SEQ ID NO: 18 with a T53C amino acid substitution; and wherein the fluorogenic small molecule is conjugated to the cysteine at that position. In certain embodiments, the nanobody comprises SEQ ID NO: 18 with a N27C amino acid substitution; and wherein the fluorogenic small molecule is conjugated to the cysteine at that position. In certain embodiments, the nanobody comprises SEQ ID NO: 18 with a T28C amino acid substitution; and wherein the fluorogenic small molecule is conjugated to the cysteine at that position. In certain embodiments, the nanobody comprises SEQ ID NO: 18 with a S30C amino acid substitution; and wherein the fluorogenic small molecule is conjugated to the cysteine at that position. In certain embodiments, the nanobody comprises SEQ ID NO: 18 with a V24C amino acid substitution; and wherein the fluorogenic small molecule is conjugated to the cysteine at that position. In certain embodiments, the nanobody comprises SEQ ID NO: 18 with a G29C amino acid substitution; and wherein the fluorogenic small molecule is conjugated to the cysteine at that position. The nanobody may include one or more additional amino acid substitutions (e.g., provided that the nanobody has at least 80% sequence identity with SEQ ID NO: 18).

In certain embodiments, the nanobody comprises one of the foregoing amino acid substitutions, further wherein one or more other cysteines of the nanobody (i.e., other than the cysteine resulting from the amino acid substitution) are independently substituted by a different amino acid (i.e., an amino acid other than cysteine). In certain embodiments, one or more other cysteines of the nanobody are independently substituted by alanine (A) or valine (V). In certain embodiments, all other cysteines of the nanobody are independently substituted by a different amino acid (i.e., an amino acid other than cysteine). In certain embodiments, all other cysteines of the nanobody are independently substituted by alanine (A) or valine (V). These nanobodies may be referred to as “no cysteine” or “noC” nanobodies.

For example, a nanobody may comprise SEQ ID NO: 18 with a T53C amino acid substitution. For example, a nanobody may comprise SEQ ID NO: 19 below (NbCor T53C):

(SEQ ID NO: 19)
QVQLQESGGGSVQAGGSLRLSCVVSGNTGSTGYWAWFRQGPGTEREGVAA
CYTAGSGTSMTYYADSVKGRFTISQDNAKKTLYLQMNSLKPEDTGMYRCA
STRFAGRWYRDSEYRAWGQGTQVTVSS.

In certain embodiments, the nanobody comprises an amino acid sequence with at least 80% sequence identity with SEQ ID NO: 19. In certain embodiments, the nanobody comprises an amino acid sequence with at least 50%, 55%, 60%, 65%, 70%, 75%, 80%, 85%, 90%, 95%, 98%, 99%, or 100% sequence identity with SEQ ID NO: 19. In certain embodiments, the nanobody comprises an amino acid sequence with about 90%, 91%, 92%, 93%, 94%, 95%, 96%, 97%, 98%, 99%, or 100% sequence identity with SEQ ID NO: 19. In certain embodiments, the nanobody comprises an amino acid sequence with 100% sequence identity with SEQ ID NO: 19.

### Mini-Proteins

In certain embodiments, the protein of the fluorogenic sensor is a mini-protein. As used herein, “mini-protein” refers to a short protein (e.g., fewer than 80, fewer than 70, fewer than 60, fewer than 50, fewer than 40 amino acids in length). In certain embodiments, the mini-protein is a synthetic protein. In certain embodiments, the mini-protein adopts a stable molecular structure. See, e.g., Baker et al., Acc. Chem. Res. vol. 50, 9, 2085-2092 (2017). One advantage of using mini-proteins as the target-binding component of the fluorogenic sensors described herein is their relatively small size. Mini-proteins can be readily synthesized (e.g., via chemical synthesis) to obtain desired sequences with custom variation.

Provided herein are fluorogenic sensors for detecting targets comprising: a mini-protein; and a fluorogenic small molecule conjugated to the mini-protein. In certain embodiments, the fluorogenic small molecule is conjugated at or around a target-binding domain of the mini-protein. In certain embodiments, the fluorogenic small molecule is conjugated to a target-binding domain of the mini-protein. In certain embodiments, the mini-protein specifically binds a target. In certain embodiments, the target is an antigen.

Provided herein are fluorogenic sensors for detecting antigens comprising: a mini-protein; and a fluorogenic small molecule conjugated to the mini-protein. In certain embodiments, the fluorogenic small molecule is conjugated at or around an antigen-binding domain of the mini-protein. In certain embodiments, the fluorogenic small molecule is conjugated to an antigen-binding domain of the mini-protein. In certain embodiments, the mini-protein specifically binds an antigen.

In certain embodiments, the mini-protein binds a pathogen (e.g., specifically binds a pathogen). In certain embodiments, the pathogen is a virus. In certain embodiments, the pathogen is a coronavirus or variant thereof. In certain embodiments, the pathogen is a SARS-CoV-2 virus or variant thereof. In certain embodiments, the pathogen is an influenza virus or variant thereof (e.g., influenza A, B, C, or D, or a variant thereof)

In certain embodiments, the mini-protein binds (e.g., specifically binds) a spike protein of a coronavirus or variant thereof. In certain embodiments, the mini-protein binds (e.g., specifically binds) a spike protein of a SARS-CoV-2 virus or variant thereof. In certain embodiments, the mini-protein binds (e.g., specifically binds) a spike protein of an influenza virus or variant thereof (e.g., influenza A, B, C, or D, or a variant thereof).

In certain embodiments, the mini-protein binds (e.g., specifically binds) a nucleocapsid protein of a coronavirus or variant thereof. In certain embodiments, the mini-protein binds (e.g., specifically binds) a nucleocapsid protein of a SARS-CoV-2 virus or variant thereof. In certain embodiments, the mini-protein binds (e.g., specifically binds) a nucleocapsid protein of an influenza virus or variant thereof (e.g., influenza A, B, C, or D, or a variant thereof).

### LCB3 Mini-Proteins

LCB3 mini-proteins specifically bind spike proteins of the SARS-CoV-2 virus and variants thereof. In certain embodiments, the nanobody comprises a LCB3 mini-protein or a fragment thereof. In certain embodiments, the nanobody comprises an amino acid sequence with at least 80% sequence identity with the sequence of LCB3 mini-protein or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a LCB3 mini-protein, or a fragment thereof. LCB3 mini-proteins comprise SEQ ID NO: 14.

In certain embodiments, the mini-protein comprises an amino acid sequence with at least 80% sequence identity with SEQ ID NO: 14:

(SEQ ID NO: 14)
NDDELHMLMTDLVYEALHFAKDEEIKKRVFQLFELADKAYKNNDRQKLEK
VVEELKELLERLLS.

In certain embodiments, the mini-protein comprises an amino acid sequence with at least 50%, 55%, 60%, 65%, 70%, 75%, 80%, 85%, 90%, 95%, 98%, 99%, or 100% sequence identity with SEQ ID NO: 14. In certain embodiments, the mini-protein comprises an amino acid sequence with about 90%, 91%, 92%, 93%, 94%, 95%, 96%, 97%, 98%, 99%, or 100% sequence identity with SEQ ID NO: 14. In certain embodiments, the mini-protein comprises an amino acid sequence with 100% sequence identity with SEQ ID NO: 14.

In certain embodiments, the fluorogenic small molecule is conjugated to an amino acid at position 19 of SEQ ID NO: 14, or a variant thereof.

In certain embodiments, the fluorogenic small molecule is conjugated to a thiol (—SH)-containing residue of the mini-protein. In certain embodiments, the fluorogenic small molecule is conjugated to a cysteine (C) residue of the mini-protein. In certain embodiments, the mini-protein comprises one or more amino acids substituted by cysteine (C) and, wherein the fluorogenic small molecule is conjugated to one of said cysteine.

For example, in certain embodiments, the mini-protein comprises SEQ ID NO: 14 with at least one amino acid substitution, wherein the at least one amino acid substitution is H19C; and wherein the fluorogenic small molecule is conjugated to the cysteine at that position.

For example, a mini-protein may comprise LCB3 H19C; (SEQ ID NO: 15 below):

(SEQ ID NO: 15)
HNDDELHMLMTDLVYEALCFAKDEEIKKRVFQLFELADKAYKNNDRQKLE
KVVEELKELLERLLS.

In certain embodiments, the mini-protein comprises an amino acid sequence with at least 80% sequence identity with SEQ ID NO: 15. In certain embodiments, the mini-protein comprises an amino acid sequence with at least 50%, 55%, 60%, 65%, 70%, 75%, 80%, 85%, 90%, 95%, 98%, 99%, or 100% sequence identity with SEQ ID NO: 15. In certain embodiments, the mini-protein comprises an amino acid sequence with about 90%, 91%, 92%, 93%, 94%, 95%, 96%, 97%, 98%, 99%, or 100% sequence identity with SEQ ID NO: 15. In certain embodiments, the mini-protein comprises an amino acid sequence with 100% sequence identity with SEQ ID NO: 14.

In certain embodiments, the mini-protein comprises one of the foregoing amino acid substitutions, further wherein one or more other cysteines of the mini-protein (i.e., other than the cysteine resulting from the amino acid substitution) are independently substituted by a different amino acid (i.e., an amino acid other than cysteine). In certain embodiments, one or more other cysteines of the mini-protein are independently substituted by alanine (A) or valine (V). In certain embodiments, all other cysteines of the mini-protein are independently substituted by a different amino acid (i.e., an amino acid other than cysteine). In certain embodiments, all other cysteines of the mini-protein are independently substituted by alanine (A) or valine (V). These mini-protein may be referred to as “no cysteine” or “noC” mini-protein.

### Fluorogenic Probes

As described herein, the fluorogenic sensors comprise a fluorogenic small molecule conjugated at or around a target-binding domain (e.g., antigen-binding domain) of the protein (e.g., nanobody or mini-protein). The fluorogenic small molecule is covalently conjugated to the protein (e.g., nanobody or mini-protein) either through a covalent bond or linker moiety.

In certain embodiments, the fluorogenic small molecule comprises one of the following formulae:

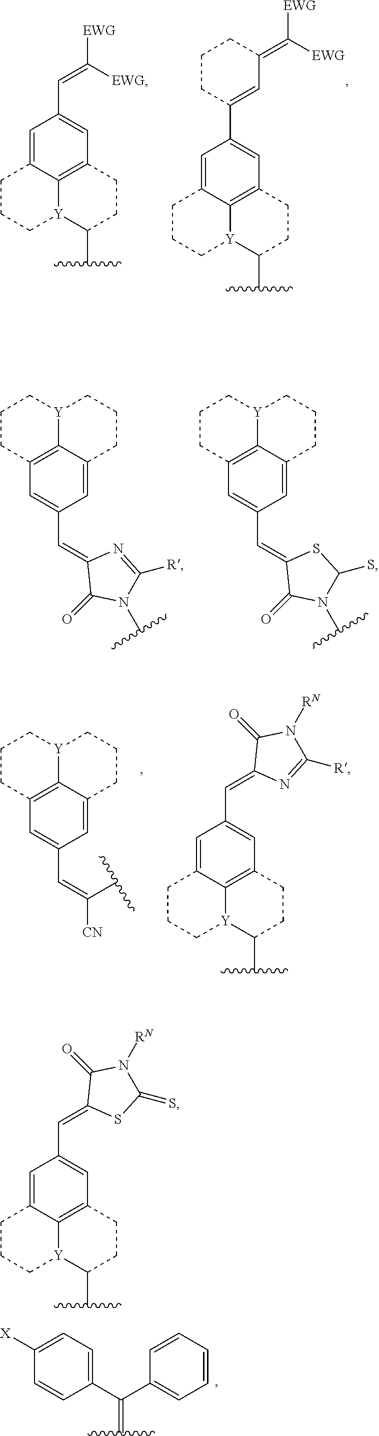

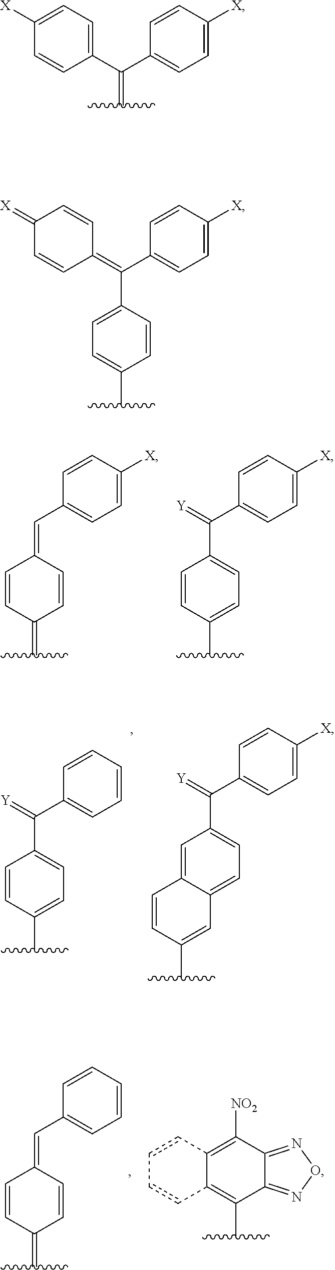

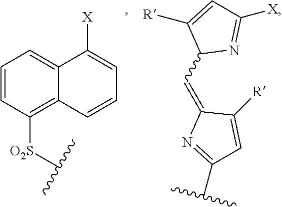

or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
   each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

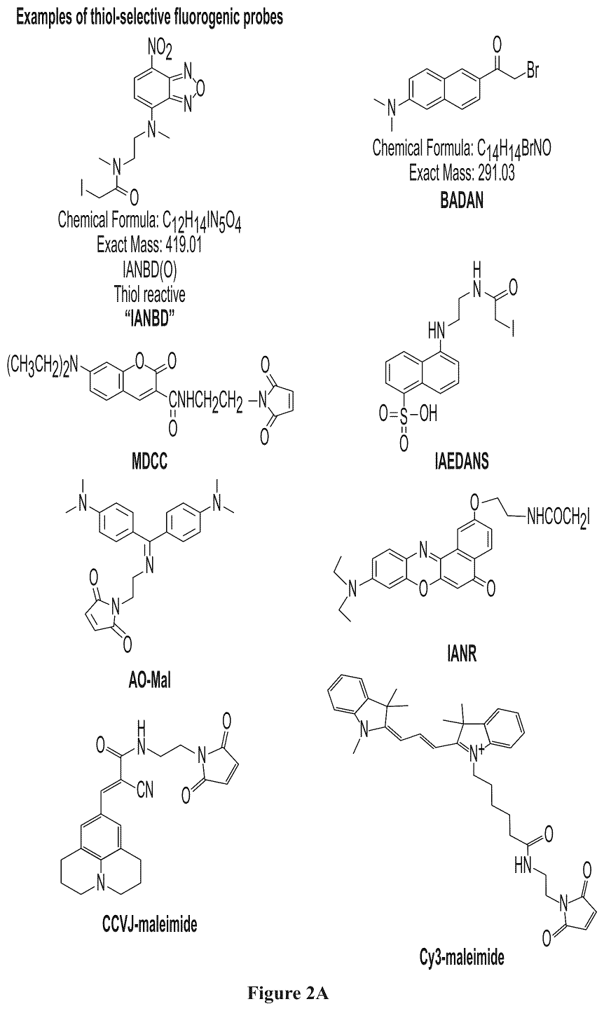

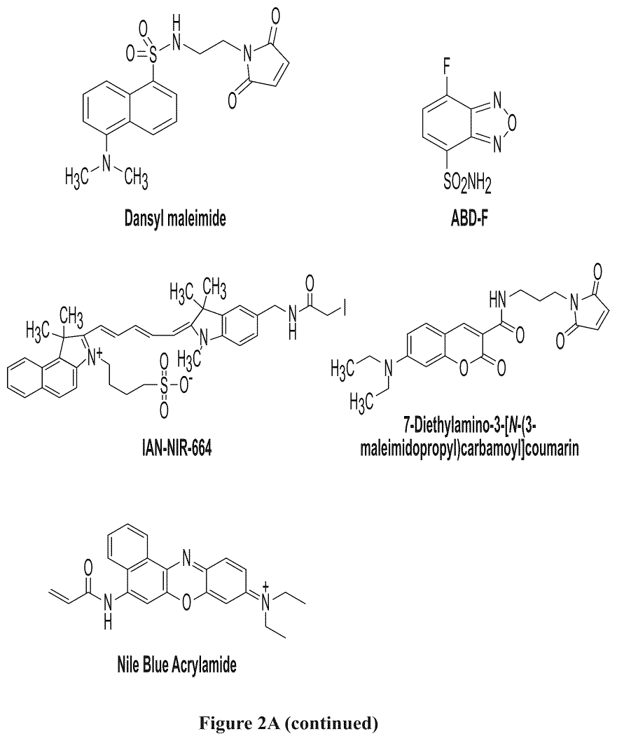

Other examples of fluorogenic small molecules can be found in, e.g., Klymchenko et al. Acc. Chem. Res. 2017, 50, 366-375; the entire contents of which is incorporated herein by reference. In certain embodiments, the fluorogenic small molecule comprises a fluorescent moiety represented in FIG. 2A or FIG. 2B.

In certain embodiments, the fluorogenic small molecule conjugated to the protein (e.g., nanobody or mini-protein) results from conjugating a compound of the following formula (i.e., “fluorogenic probe”) to the protein:

FG-L-A,

or a salt, stereoisomer, or tautomer thereof; wherein FG is the fluorogenic small molecule; L is a bond or a linker; and A is a reactive moiety.

In certain embodiments, FG is of one of the following formulae:

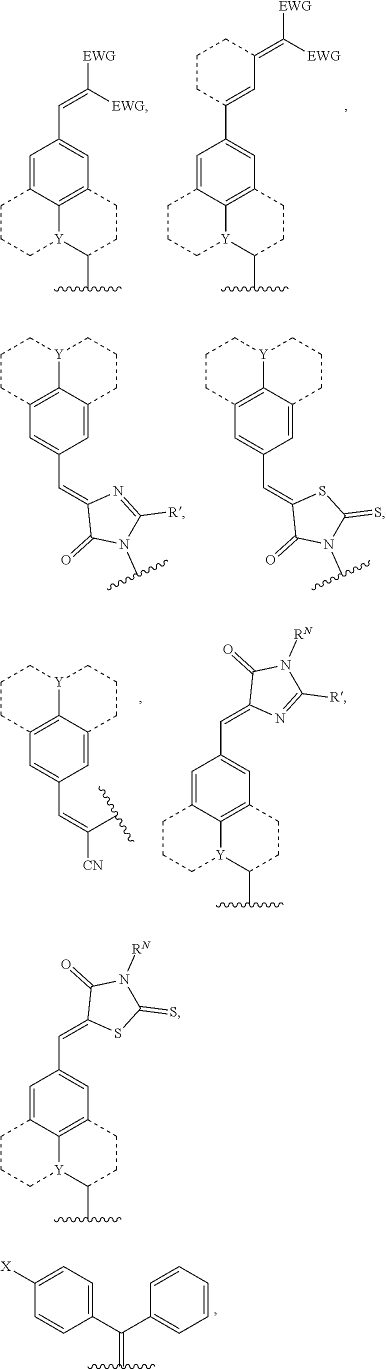

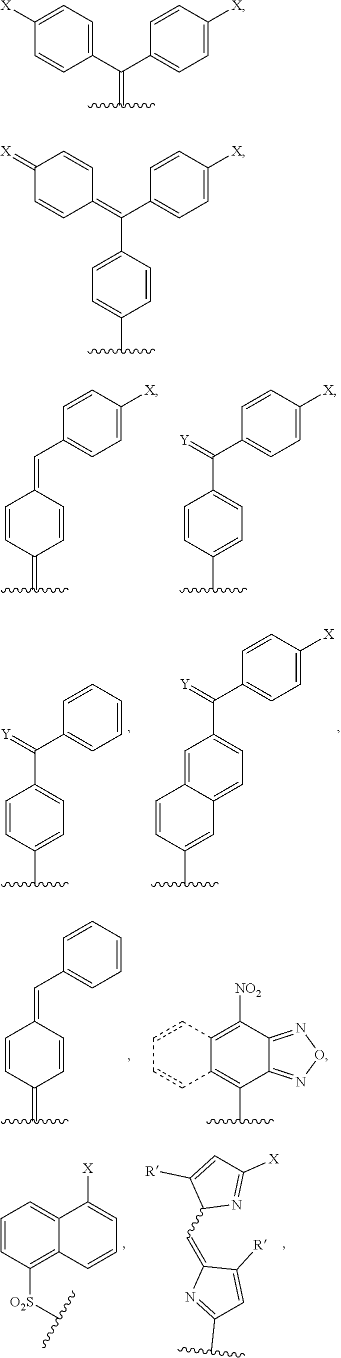

or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

In certain embodiments, the group -L-A is of one of the following formulae:

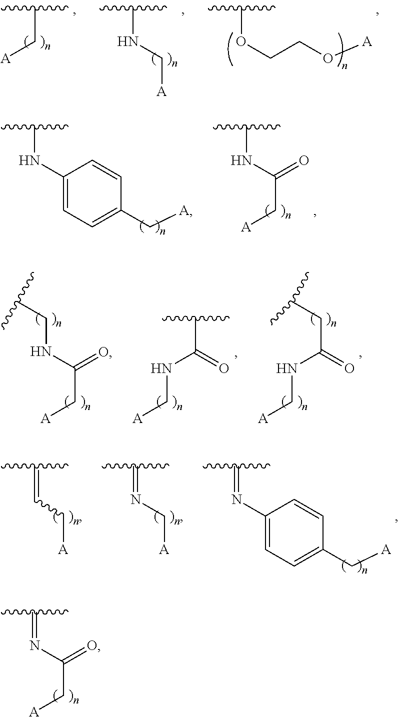

or a salt, stereoisomer, or tautomer thereof; wherein:
each n is independently 0 or an integer from 1-20, inclusive; and
wherein each formula is further optionally substituted.

As described herein, A is a reactive moiety. In certain embodiments, A is a lysine- or cysteine-selective reactive moiety. In certain embodiments A is a lysine-selective reactive moiety. In certain embodiments, A is a cysteine-selective reactive moiety.

For the purposes of this disclosure, a “reactive moiety” is any chemical moiety capable of reacting with another chemical moiety to form a covalent bond or covalent bonds. Non-limiting examples of reactive moieties include alkenes, alkynes, alcohols, amines, thiols, azides, esters, amides, halogens, and the like. In certain embodiments, two reactive moieties are capable of reacting directly with each other to form one or more covalent bonds. In other embodiments, two reactive moieties react with an intervening linking reagent to form a covalent linkage. In certain embodiments, the reactive moieties are click chemistry moieties. “Click chemistry” moieties are any moieties that can be used in click chemistry reactions.

“Click chemistry” is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395. Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition). As an example, in the case of reactions between an azide and alkyne reactive moieties to form triazolylene linkages, alkyne-azide 1,3-cycloadditions may be used (e.g., the Huisgen alkyne-azide cycloaddition). In certain embodiments, the alkyne-azide cycloaddition is copper-catalyzed. In certain embodiments, the alkyne-azide cycloaddition is strain-promoted. Examples of alkyne-azide reactions can be found in, e.g., Kolb, Finn and Sharpless Angewandte Chemie International Edition (2001) 40: 2004-2021; Kolb and Sharpless, Drug Discov Today (2003) 24: 1128-1137; and Evans, Australian Journal of Chemistry (2007) 60: 384-395.

In certain embodiments, A comprises a halogen, alkene, alkyne, azide, tetrazine, or a moiety of one of the following formulae:

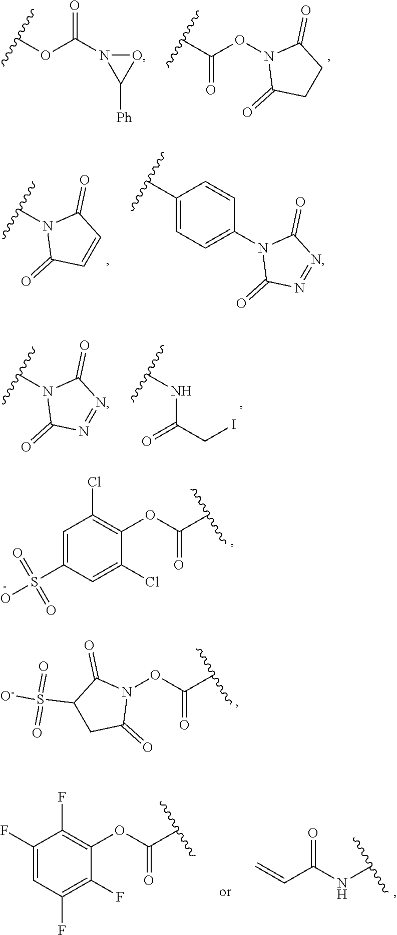

or a salt, stereoisomer, or tautomer thereof; wherein each formula is further optionally substituted.

The table below shows the reactive moieties and their associated chemoselectivity.

| Reactive Moiety | Chemoselectivity |
|---|---|
| azide | alkynes |
| alkyne | azides |
| 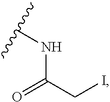 [SMILES: *NC(=O)CI] | thiols (e.g., cysteine) |
| 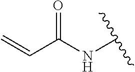 [SMILES: *NC(=O)C=C] | |
| 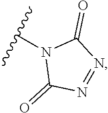 [SMILES: *N1C(=O)N=NC1=O] | phenols (e.g., tyrosine) |
| 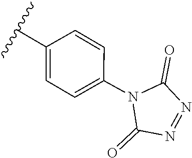 [SMILES: *c1ccc(N2C(=O)N=NC2=O)cc1] | |
| 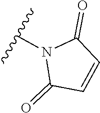 [SMILES: *N1C(=O)C=CC1=O] | thiols (e.g., cysteine) |
| 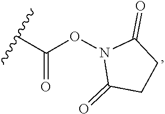 [SMILES: *C(=O)ON1C(=O)CCC1=O] | amines (e.g., lysine) |
| 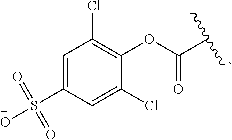 [SMILES: *C(=O)Oc1c(Cl)cc(S(=O)(=O)[O-])cc1Cl] | |
| 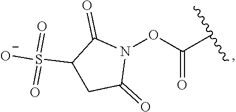 [SMILES: *C(=O)ON1C(=O)CC(S(=O)(=O)[O-])C1=O] | |
| 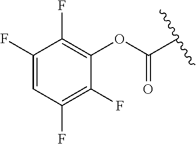 [SMILES: *C(=O)Oc1c(F)c(F)cc(F)c1F] | |
| 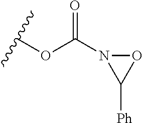 [SMILES: *OC(=O)N1OC1c1ccccc1] | thioethers (e.g., methionine) |

The present disclosure includes any of the foregoing fluorogenic probes (including any and all possible combinations of FG, L, and A) as part of the fluorogenic sensors described herein (i.e., conjugated to an antigen-binding protein (e.g., nanobody or mini-protein)), and also as compounds (i.e., not conjugated to a protein). In certain embodiments, the fluorogenic probe is selected from those provided in FIG. 2A and FIG. 2B.

### Additional Fluorogenic Probes

The present disclosure also provides the following compounds which may be used as fluorogenic probes (e.g., for conjugation to a protein (e.g., nanobody or mini-protein) form a fluorogenic sensor described herein). In certain embodiments, the compounds described have amine- or thiol-selective reactivity.

Provided herein are compounds of the following formula:

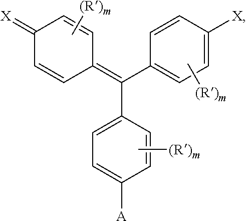

and salts, stereoisomers, and tautomers thereof, wherein:

A is a reactive moiety comprising one of the following formulae:

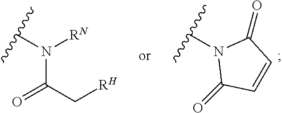

R^{H} is halogen or a leaving group;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

In certain embodiments, the compound is selected from the group consisting of:

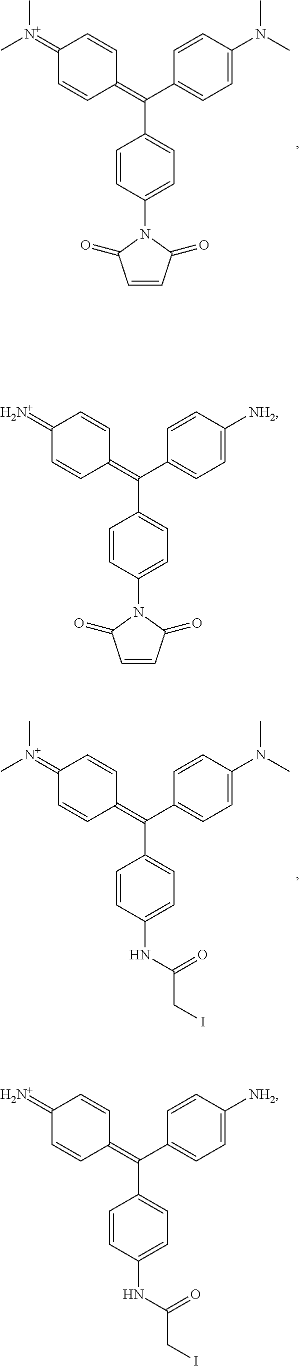

and salts, stereoisomers, and tautomers thereof.

Also provided herein are compounds of the following formula:

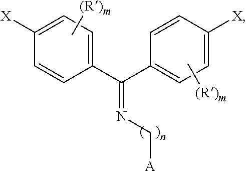

and salts, stereoisomers, and tautomers thereof, wherein:
A is a reactive moiety;
each instance of X is independently —N(R^{N})₂, —OR^{O}, or —SR^{S};
n is 0 or an integer from 1-20, inclusive.
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4;
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

In certain embodiments, A comprises a halogen, alkene, alkyne, azide, tetrazine, or a moiety of one of the following formulae:

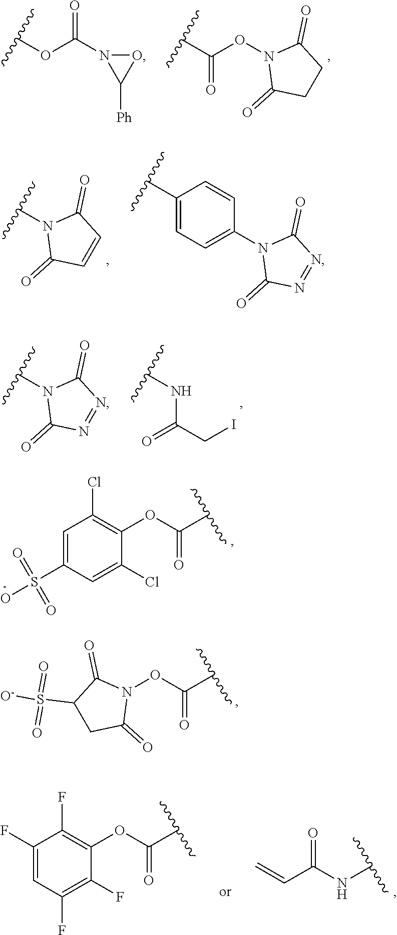

or a salt, stereoisomer, or tautomer thereof; wherein each formula is further optionally substituted.

In certain embodiments, the compound is of the formula:

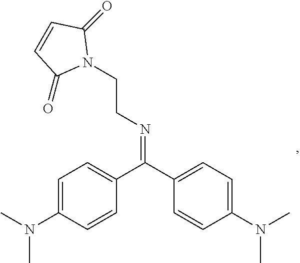

or a salt or tautomer thereof.

Also provided herein are compounds of the following formula:

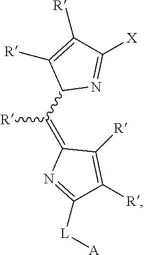

and salts, stereoisomers, and tautomers thereof; wherein:
L is a bond or a linker;
A is a reactive moiety;
each instance of R′ and X is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

In certain embodiments, the compound is of the following formula:

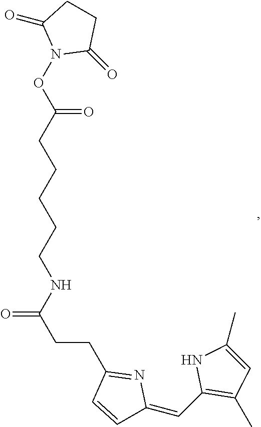 [SMILES: Cc1cc(C)c(/C=C2/C=CC(CCC(=O)NCCCCCC(=O)ON3C(=O)CCC3=O)=N2)[nH]1]

or a salt or tautomer thereof.

Also provided herein are compounds of the following formulae:

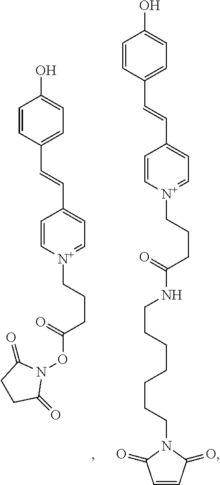 [SMILES: O=C(CCC[n+]1ccc(/C=C/c2ccc(O)cc2)cc1)ON1C(=O)CCC1=O ; O=C(CCC[n+]1ccc(/C=C/c2ccc(O)cc2)cc1)NCCCCCCCN1C(=O)C=CC1=O]

and pharmaceutically acceptable salts and tautomers thereof; wherein the compound is further optionally substituted.

Also provided herein are compounds of the following formula:

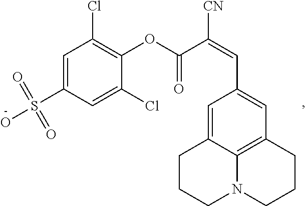 [SMILES: N#C/C(=C/c1cc2c3c(c1)CCCN3CCC2)C(=O)Oc1c(Cl)cc(S(=O)(=O)O)cc1Cl]

and salts, stereoisomers, and tautomers thereof; wherein the compound is further optionally substituted.

### Fluorogenic Amino Acids

As described herein, in certain embodiments, a fluorogenic sensor results from conjugating a fluorogenic probe comprising a reactive moiety at or around a target-binding domain (e.g., antigen-binding domain) of a protein (e.g., nanobody or mini-protein). In other embodiments, a binding domain of the protein (e.g., nanobody or mini-protein) comprises an unnatural amino acid comprising a fluorogenic small molecule (i.e., “fluorogenic amino acid” or “FgAA”). In preferred embodiments, the fluorogenic small molecule is attached to the α-position of the FgAA (e.g., through a covalent bond or a linker moiety).

In certain embodiments, at least one amino acid of the amino sequence of the protein (e.g., nanobody or mini-protein) is substituted by a FgAA. For example, a FgAA can be encoded into the amino acid sequence of the protein (e.g., nanobody or mini-protein) or installed via transpeptidation. In certain embodiments, the FgAA is encoded into the amino acid sequence of the protein (e.g., nanobody or mini-protein) via ribosomal synthesis. The protein (e.g., nanobody or mini-protein) can also be chemically synthesized (e.g., via solid-phase peptide synthesis), with FgAAs incorporated at one or more positions.

For example, in certain embodiments, the nanobody comprises SEQ ID NO: 1 or 2 with at least one of the following amino acids substituted by a FgAA: I51, W53, G56, Y59, G102, T103, V104, V105, W108, Y110, and W115. In certain embodiments, the nanobody comprises SEQ ID NO: 1 or 2 with a I51(FgAA) amino acid substitution. In certain embodiments, the nanobody comprises SEQ ID NO: 1 or 2 with a W53(FgAA) amino acid substitution. In certain embodiments, the nanobody comprises SEQ ID NO: 1 or 2 with a G56(FgAA) amino acid substitution. In certain embodiments, the nanobody comprises SEQ ID NO: 1 or 2 with a Y59(FgAA) amino acid substitution. In certain embodiments, the nanobody comprises SEQ ID NO: 1 or 2 with a G102(FgAA) amino acid substitution. In certain embodiments, the nanobody comprises SEQ ID NO: 1 or 2 with a T103(FgAA) amino acid substitution. In certain embodiments, the nanobody comprises SEQ ID NO: 1 or 2 with a V104(FgAA) amino acid substitution. In certain embodiments, the nanobody comprises SEQ ID NO: 1 or 2 with a V105(FgAA) amino acid substitution. In certain embodiments, the nanobody comprises SEQ ID NO: 1 or 2 with a W108(FgAA) amino acid substitution. In certain embodiments, the nanobody comprises SEQ ID NO: 1 or 2 with a Y110(FgAA) amino acid substitution. In certain embodiments, the nanobody comprises SEQ ID NO: 1 or 2 with a W115(FgAA) amino acid substitution. The nanobody may include one or more additional amino acid substitutions (e.g., provided that the nanobody has at least 80% sequence identity with SEQ ID NO: 1 or 2).

For example, in certain embodiments, the nanobody comprises SEQ ID NO: 10 with a F29(FgAA) amino acid substitution. In certain embodiments, the nanobody comprises SEQ ID NO: 12 with a K65(FgAA) amino acid substitution. In certain embodiments, the nanobody comprises SEQ ID NO: 16 with a M63(FgAA) amino acid substitution. In certain embodiments, a mini-protein comprises SEQ ID NO: 14 with a H19(FgAA) amino acid substitution. In certain embodiments, a nanobody comprises SEQ ID NO: 18 with at least one amino acid substitution selected from V24(FgAA), A78(FgAA), K79(FgAA), T53(FgAA), N27(FgAA), T28(FgAA), S30(FgAA), V24(FgAA), G29(FgAA), and W34(FgAA). The nanobody or mini-protein may include one or more additional amino acid substitutions (e.g., provided that the nanobody or mini-protein has at least 80% sequence identity with the recited amino acid sequence).

In certain embodiments, a fluorogenic amino acid comprises any one of the formulae provided for -FG (supra). In certain embodiments, a fluorogenic amino acid comprises any one of the fluorogenic moieties represented in FIG. 2A and FIG. 2B. Examples of fluorogenic amino acids which are considered part of the present disclosure can be found in, e.g., International PCT Application Publication WO 2021/118727, published Jun. 17, 2021, the entire contents of which is incorporated herein by reference.

For example, in certain embodiments, the FgAA is of one of the following formulae:

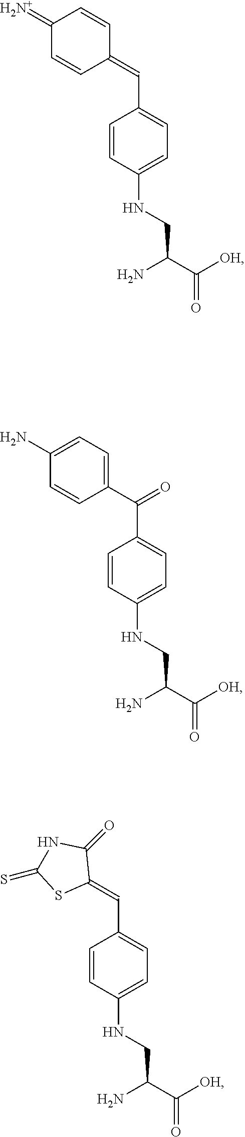 [SMILES: N[C@@H](CNc1ccc(C=C2C=CC(=[NH2+])C=C2)cc1)C(=O)O ; Nc1ccc(C(=O)c2ccc(NC[C@H](N)C(=O)O)cc2)cc1 ; N[C@@H](CNc1ccc(/C=C2\SC(=S)NC2=O)cc1)C(=O)O]

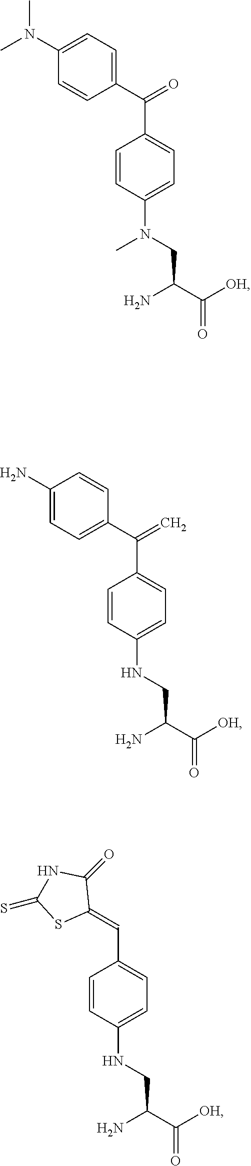 [SMILES: CN(C)c1ccc(C(=O)c2ccc(N(C)C[C@H](N)C(=O)O)cc2)cc1 ; C=C(c1ccc(N)cc1)c1ccc(NC[C@H](N)C(=O)O)cc1 ; N[C@@H](CNc1ccc(/C=C2\SC(=S)NC2=O)cc1)C(=O)O]

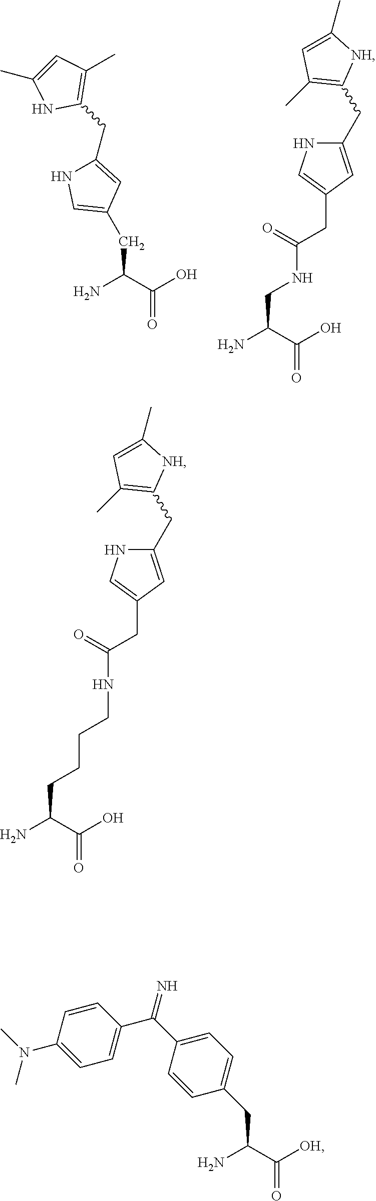 [SMILES: Cc1cc(C)c(Cc2cc(C[C@H](N)C(=O)O)c[nH]2)[nH]1 ; Cc1cc(C)c(Cc2cc(CC(=O)NC[C@H](N)C(=O)O)c[nH]2)[nH]1 ; Cc1cc(C)c(Cc2cc(CC(=O)NCCCC[C@H](N)C(=O)O)c[nH]2)[nH]1 ; CN(C)c1ccc(C(=N)c2ccc(C[C@H](N)C(=O)O)cc2)cc1]

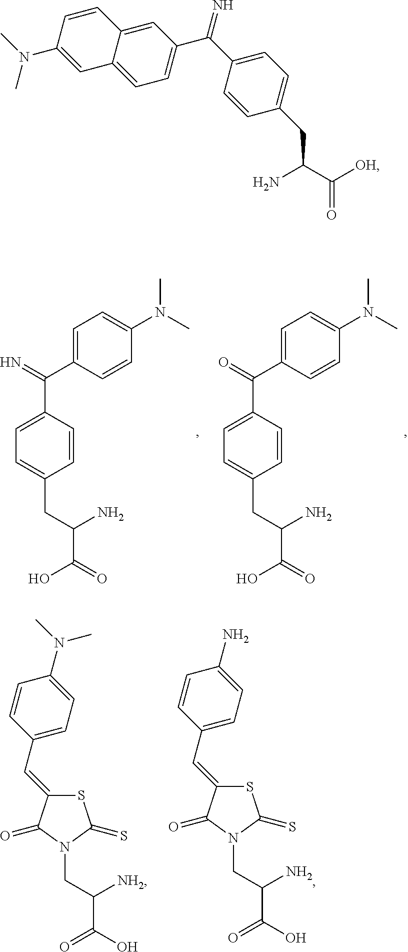

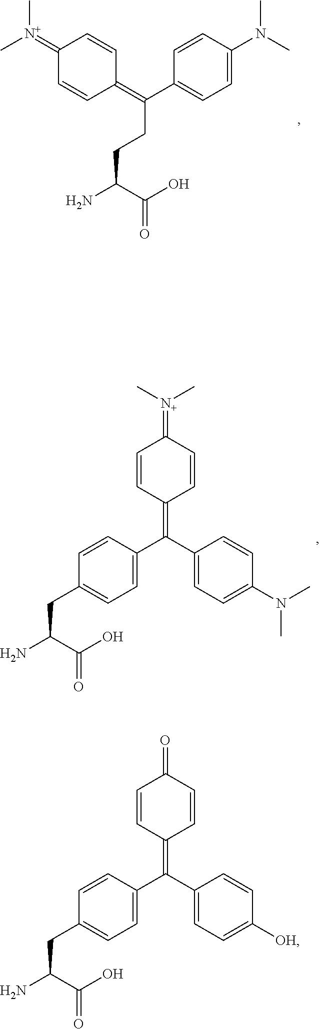

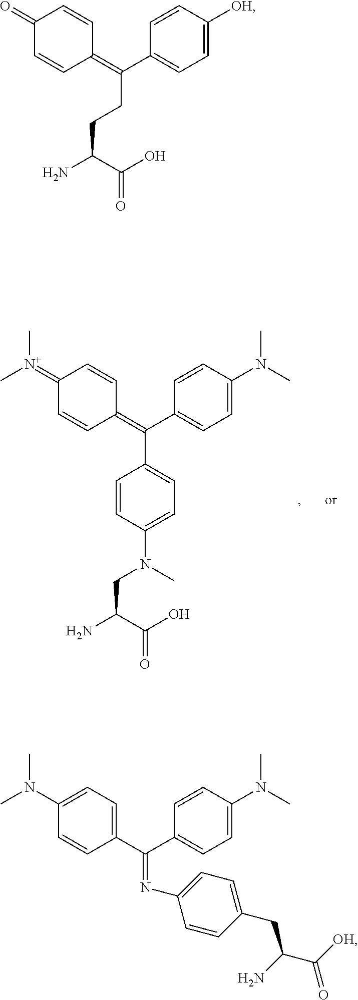

or a salt, stereoisomer, or tautomer thereof.

### N-Terminus Modifications

In certain embodiments, a fluorogenic sensor provided herein further comprises a second fluorogenic small molecule conjugated to the N-terminus of the protein (e.g., nanobody). In certain embodiments, the first fluorogenic small molecule (i.e., the fluorogenic small molecule conjugated to a binding domain of the protein (e.g., nanobody)) and the second fluorogenic small molecule are a fluorescence resonance energy transfer (FRET) pair.

“FRET” is a physical phenomenon in which a first fluorophore in its excited state (i.e., donor fluorophore) non-radiatively transfers its excitation energy to a second fluorophore (i.e., acceptor), thereby causing the acceptor to emit its characteristic fluorescence. FRET synergy between the first fluorogenic small molecule at the binding domain of the protein (e.g., nanobody) and the second fluorogenic small molecule at the N-terminus can lead to enhanced fluorescence and detection of antigens.

In certain embodiments, the second fluorogenic small molecule is tetramethylrhodamine (TMR). In certain embodiments, the first fluorogenic small molecule is NBD, and the second fluorogenic small molecule is TMR.

### Methods and Kits for Detecting Antigens

As described herein, the fluorogenic sensors can be used to detect protein-target interactions, and can therefore be used to detect the presence of a target (e.g., an antigen).

Provided herein are methods of determining the presence of target in a sample, the method comprising: (i) contacting a sample with a fluorogenic sensor provided herein; and (ii.a) measuring or observing the fluorescence of the sample or (ii.b) measuring or observing the change in fluorescence lifetime of the sample. As described herein, the fluorescence of the sample may increase upon binding of the fluorogenic sensor to the target. Therefore, any increase in fluorescence may be indicative of the presence of the target. In certain embodiments, the fluorescence lifetime of the sample may change upon binding of the fluorogenic sensor to the target.

As described herein, the fluorogenic sensors can be used to detect the presence of antigens. Provided herein are methods of determining the presence of an antigen in a sample, the method comprising: (i) contacting a sample with a fluorogenic sensor provided herein; and (ii.a) measuring or observing the fluorescence of the sample or (ii.b) measuring or observing the change in fluorescence lifetime of the sample. As described herein, the fluorescence of the sample may increase upon binding of the fluorogenic sensor to the antigen. Therefore, any increase in fluorescence may be indicative of the presence of the antigen. In certain embodiments, the fluorescence lifetime of the sample may change upon binding of the fluorogenic sensor to the target.

In certain embodiments, the fluorescence of the sample is increased by at least 10%. In certain embodiments, the fluorescence of the sample is increased by at least 10%, 20%, 30%, 40%, 50%, 60%, 70%, 80%, or 90%. In certain embodiments, the fluorescence of the sample is increased by at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold. In certain embodiments, the increase in fluorescence is greater than 500-fold. In certain embodiments, the fluorescence of the sample is increased by at about 5- to about 25-fold. In certain embodiments, the fluorescence of the sample is increased by at about 5- to about 100-fold. In certain embodiments, the fluorescence of the sample is increased by at about 5- to about 50-fold. In certain embodiments, the fluorescence of the sample is increased by at at least 100-fold.

In certain embodiments, the fluorescence of the sample is decreased by at least 10%. In certain embodiments, the fluorescence of the sample is decreased by at least 10%, 20%, 30%, 40%, 50%, 60%, 70%, 80%, or 90%.

Provided herein are methods of detecting a target, the method comprising: (i) contacting the target with a fluorogenic sensor provided herein; and (ii.a) measuring or observing the fluorescence of the fluorogenic sensor or (ii) measuring or observing the change in fluorescence lifetime of the fluorogenic sensor. As described herein, the fluorescence of the sample may increase upon binding of the fluorogenic sensor to the target. In certain embodiments, the fluorescence lifetime of the fluorogenic sensor may change upon binding of the fluorogenic sensor to the target. In certain embodiments, this is possible without the need to add additional components (i.e., FRET donor/accepter), an advantage over previous sensors.

Provided herein are methods of detecting an antigen, the method comprising: (i) contacting the antigen with a fluorogenic sensor provided herein; and (ii.a) measuring or observing the fluorescence of the fluorogenic sensor or (ii.b) measuring or observing the change in fluorescence lifetime of the fluorogenic sensor.

In certain embodiments, the fluorescence is increased by at least 10% upon binding to the target (e.g., antigen). In certain embodiments, the fluorescence is increased by at least 10%, 20%, 30%, 40%, 50%, 60%, 70%, 80%, or 90% upon binding to the target (e.g., antigen). In certain embodiments, the fluorescence of the sensor is increased by at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold. In certain embodiments, the increase in fluorescence is greater than 500-fold.

In certain embodiments, the fluorescence is decreased by at least 10% upon binding to the target (e.g., antigen). In certain embodiments, the fluorescence is decreased by at least 10%, 20%, 30%, 40%, 50%, 60%, 70%, 80%, or 90% upon binding to the target (e.g., antigen).

Fluorescence can be measured or observed by means known in the art. For example, in certain embodiments, the fluorescence is measured or observed by fluorescence spectroscopy (e.g., using a fluorometer). In certain embodiments, the fluorescence is observed by microscopy. In certain embodiments, the fluorescence is observed visually (e.g., with the naked eye). In certain embodiments, the detection is colorimetric.

Methods provided herein allow for rapid (e.g., instantaneous) detection of targets (e.g., antigens). In certain embodiments, an increase in fluorescence is observed within under 1 second of the contacting step. In certain embodiments, an increase in fluorescence is observed within under 2500, 2000, 1500, 1000, 750, 500, or 250 milliseconds (ms) of the contacting step. In certain embodiments, an increase in fluorescence is observed within under 2500 ms of the contacting step. In certain embodiments, an increase in fluorescence is observed within under 2000 ms of the contacting step. In certain embodiments, an increase in fluorescence is observed within under 1000 ms of the contacting step. In certain embodiments, an increase in fluorescence is observed within under 500 ms of the contacting step. In certain embodiments, an increase in fluorescence is observed within under 250 ms of the contacting step. In certain embodiments, an increase in fluorescence is observed within under 100 ms of the contacting step.

Rapid (e.g., instantaneous) detection of targets (e.g., antigens) can allow for diagnostic methods with little to no significant wait time. This includes rapid (e.g., instantaneous) detection of SARS-CoV-2 viruses, influenza viruses, and other pathogens such as bacteria. The methods also allow for rapid (e.g., instantaneous) detection of targets in other time-sensitive settings, such as during surgery or operation. Therefore, the methods described herein have intraoperation surgical application such as intraoperative specific staining to detect certain biomarkers during surgery.

In-situ detection of targets can allow for instant detection of an analyte across a variety of settings including rapid identification of food spoilage in a warehouse, or instant detection of controlled substances in a law enforcement or military setting.

In certain embodiments, the antigen to be detected is a pathogen. In certain embodiments, the pathogen is a virus. In certain embodiments, the virus is a coronavirus or variant thereof. In certain embodiments, the virus is a SARS-CoV-2 virus or a variant thereof. In certain embodiments, the SARS-CoV-2 variant is the Alpha, Beta, Dela, Gamma, or Omicron variant. In certain embodiments, the SARS-CoV-2 variant is a future variant (i.e., a variant not yet discovered or in existence). In certain embodiments, the antigen is protein present on a bacterial cell. In certain embodiments, the antigen is ALFA-tag (e.g., ALFA-tag present on a bacterial cell).

In certain embodiments, the target to be detected is a small molecule (e.g., an endogenous small molecule). In certain embodiments, the target to be detected is cortisol.

Also provided herein are kits comprising a fluorogenic sensor provided herein. In certain embodiments, the kit is useful for detecting a pathogen (e.g., virus, e.g., SARS-CoV-2 or a variant thereof) according to a method described herein. Optionally, a kit provided herein will include instructions for use.

### EXAMPLES

### Fluorogenic Sensors

Described herein is a modular platform to transform protein binders into nanosensors by leveraging fluorogenic probes (e.g., conjugatable fluorogenic probes and genetically encodable fluorogenic probes). Demonstrating the generic applicability of the platform, shown herein is the construction of nanosensors for SARS-CoV-2 antigens including the Omicron variant with ratiometric readouts up to two orders of magnitude and subsecond kinetics. This platform allows for rapid engineering of biosensors (e.g., optical biosensors) with direct applications in diagnostics and biomedical research.

Fast development of rapid, simple, and cheap tests is important to managing disease outbreaks. Reverse transcription polymerase chain reaction (RT-PCR)-based SARS-CoV-2 tests are sensitive, but they are instrumentation heavy and can give false-positive results. See, e.g., Sills et al., Science vol. 371, 244-245 (2021). Complementing these, optical biosensors can serve as antigen detection assays in real time, often requiring only a binding interaction and abrogating the need for multiple components while also diversifying the testing supply chain. See, e.g., Mercer et al., Nature Reviews Genetics vol. 22, 415-426 (2021).

Modification of protein binders with environmentally-sensitive fluorophores (or fluorogens) can transform them into optical biosensors that generate an easily detectable readout upon target binding. See, e.g., de Picciotto et al., Journal of Molecular Biology vol. 428, 4228-4241 (2016); Dong et al., Sensors (Basel) vol. 21, 1223 (2021); Brient-Litzler et al., Protein Engineering, Design and Selection vol. 23, 229-241 (2009); Mills et al., ChemBioChem vol. 10, 2162-2164 (2009); and De Lorimier et al., Protein Sci. vol. 11, 2655-2675 (2002). A variety of robust technologies now exist that can rapidly evolve specific protein binders against a plethora of targets, such as compact single domain antibodies (or nanobodies) or “mini-proteins” against the SARS-CoV-2 spike protein. See, e.g., Wrapp et al., Cell vol. 181, 1004-1015 (2020); and Cao et al., Science vol. 370, 426-431 (2020). However, currently validated optical biosensors generally respond to their targets poorly. See, e.g., Adamson et al., ACS Sensors vol. 5, 3001-3012 (2020).

With an initial focus on (<15 kDa) protein-binders primarily against SARS-CoV-2 antigens, instant nanosensors were rapidly engineered using chemical conjugation and synthetic biology approaches. See, e.g., Wrapp et al., Cell vol. 181, 1004-1015 (2020); Cao et al., Science vol. 370, 426-431 (2020); Götzke et al., Nature Communications vol. 10, 4403 (2019); Huo et al., Nature structural & molecular biology vol. 27, 846-854 (2020); and Ye et al., Front. Immunol. vol. 12, 719037 (2021).

For example, one approach relies on the modular and simple derivatization of protein binders with cysteine- or lysine-reactive fluorogenic probes (fluorogenic small molecules) under cost-effective and scalable (>25 mg from 1 L E. coli culture) sensor manufacturing procedures. The multiplexed exploration of various fluorogen-spacer-position combinations can streamline the nanosensor discovery. Another approach relies on ribosomal construction (e.g., cell-free ribosomal construction) of fluorogenic nanosensors These approaches allow for universal conversion of small protein binders into optical sensors for numerous bioimaging and analytical applications.

### Example 1: Development of Nanobody and Mini-Protein Sensors of SARS-CoV-2

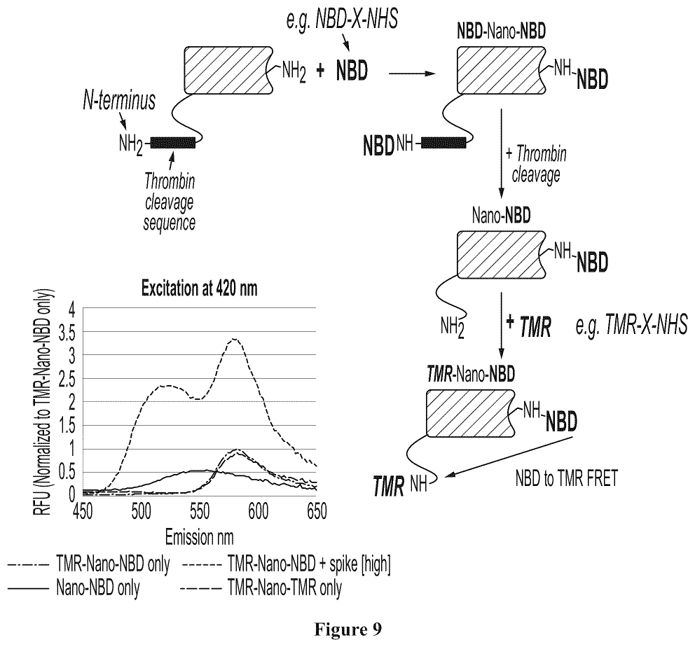

Leveraging fluorogenic small molecules, fluorogenic amino acids, chemical conjugation technologies, and genetic code expansion technologies, new protein biosensors for SARS-CoV-2 antigens were engineered. See, e.g., Kuru et al., ACS Chemical Biology vol. 15, 1852-1861 (2020); and Cheng et al., Nature Reviews Chemistry vol. 4, 275-290 (2020). Antibodies labeled with L-(7-hydroxycoumarin-4-yl) ethylglycine (Cou), a genetically-encodable fluorescent amino acid, showed a small fluorescence increase upon target binding. See, e.g., Mills et al., ChemBioChem vol. 10, 2162-2164 (2009). Following the same amber suppression approach and aided by the high-resolution crystal structure of an anti-SARS-CoV-2 spike receptor binding domain (RBD) nanobody VHH72, a recombinant VHH72 W108Cou variant was prepared, substituting a binding interface tryptophan for Cou. See, e.g., Wrapp et al., Cell vol. 181, 1004-1015 (2020). This variant showed a modest fluorescence change in the presence of saturating SARS-CoV-2 spike protein receptor-binding domain (RBD) (FIG. 10).

The optimization of the fluorogen, spacer, and residue combination remains the main challenge in fluorogenic protein sensor discovery. Despite the faster and irreversible reactivities of amine-reactive reagents, chemoselective protein modification approaches typically exclude them due to the natural abundance of lysines in proteins and focus on thiol-reactive reagents instead. See, e.g., Hoyt et al., Nature Reviews Chemistry vol. 3, 147-171 (2019). To expand the discovery approach also to amine-reactive probes, the four nanobody framework lysines of VHH72 were mutated to isoelectric arginines (K43R, K65R, K76R, K87R) (“VHH72 noK”). It was confirmed that the VHH72 noK variant did not show a decreased affinity for RBD. This improved flexibility enables multiplexed screening of lysine- and cysteine-functionalized nanobody variant-dye combinations. Specifically, eleven VHH72+C and six VHH72 noK+K variants that sample artificially introduced cysteine/lysine residues at or around the binding interface were purified. They were then chemoselectively modified in a 96-well format, with a set of different 8 thiol-reactive or 7 lysine-reactive dyes and determined the fluorescence fold increase (ΔRmax) in the presence of RBD. This approach allowed the screening of fluorophores with variable properties, including different fluorogenicity (e.g., fluorescent tetramethylrhodamine vs. fluorogenic Malachite Green), emission wavelength (e.g., green fluorescent IANBD vs. blue fluorescent MDCC), spacers (e.g., NBD-hexanoate vs. NBD-dodecanoate), reactivities (e.g., maleimides or iodoacetamides vs. NHS-esters or isothiocyanates), and new reactive fluorogens (e.g., molecular rotors IAMG or AO-Mal, and the solvatochromic APM-octa-NHS). See, e.g., Babendure et al., JACS vol. 125, 14716-14717 (2003); Benson, et al., Nat. Commun. vol. 12, 2369 (2021); Erez et al., The Journal of Physical Chemistry A vol. 116, 12056-12064 (2012); and Cohen et al., Proceedings of the National Academy of Sciences of the United States of America vol. 102, 965-970 (2005).

The screen revealed several VHH72 variant-probe combinations enriched around “hot” interface positions resulting in significant fluorescence fold-increases. The highest fold increases were observed for VHH72 G56C or V104C modified with MDCC and VHH72 noK V104K modified with NBDx-NHS. The robust fluorescence response combined with the convenience of the NHS-chemistry led to further characterization of VHH72 noK V104NBDxK (VHH72noK V104K functionalized with NBDx-NHS at the V104K position; “nanoX”).

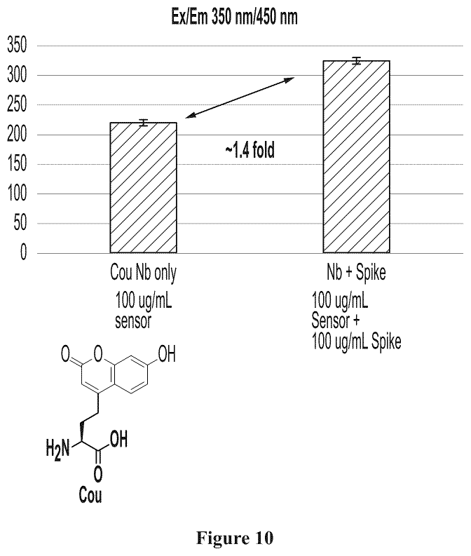

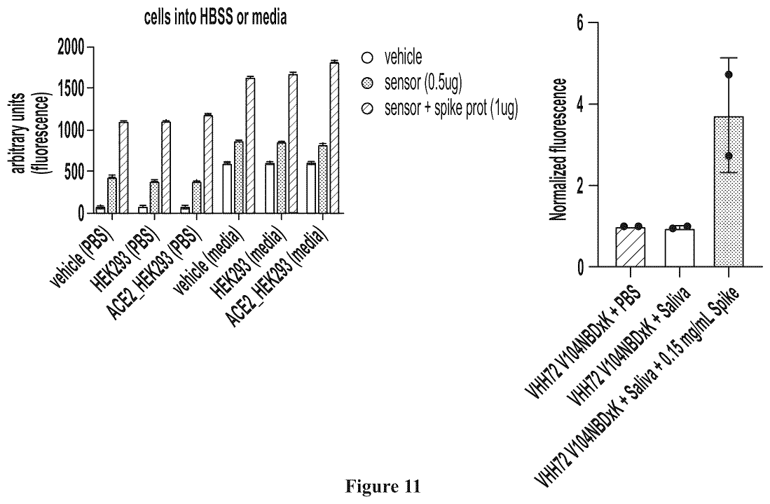

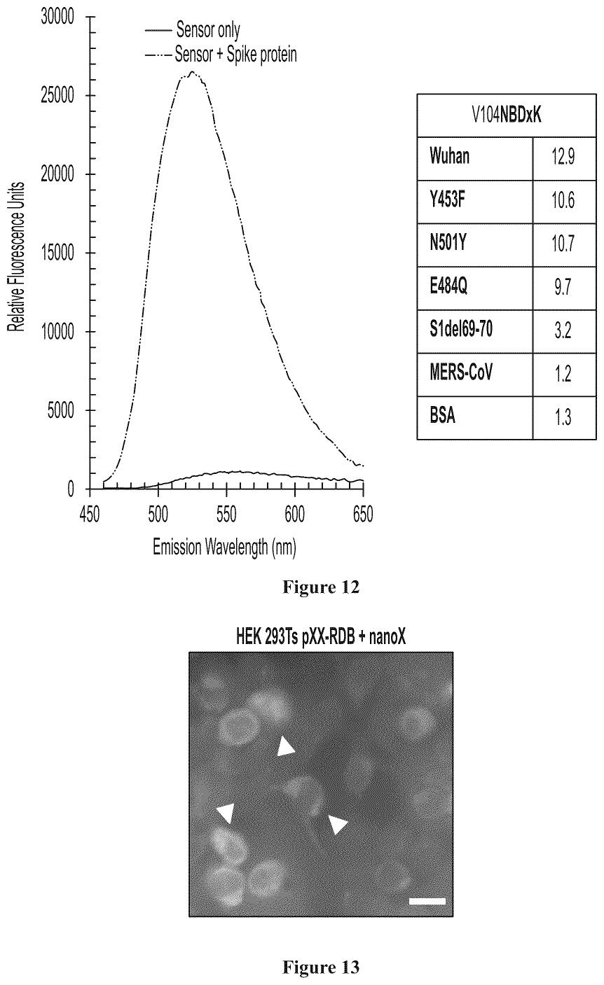

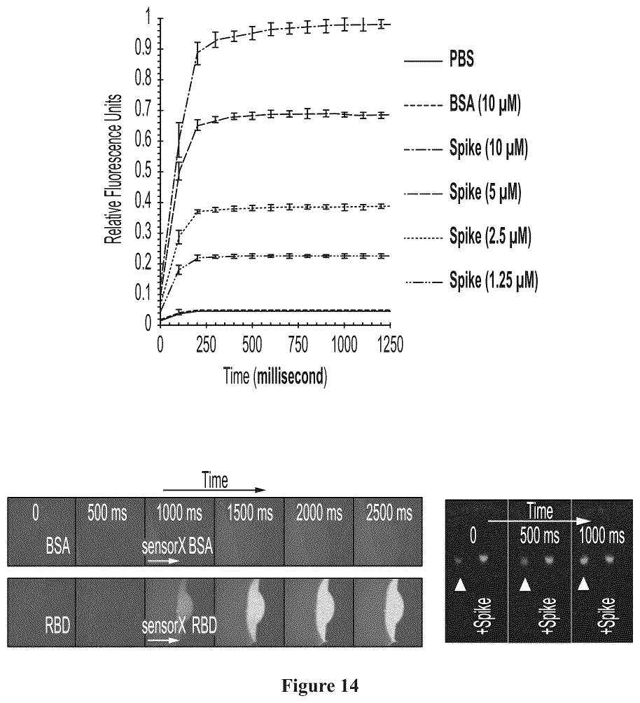

After confirming the NBDx modification at the V104K position by MS, it was confirmed that this nanosensor could selectively respond to the spike protein even in complex environments (FIG. 11) by increasing its fluorescence emission up to 100-fold, a significant fluorescence increase that is rarely observed in biosensors (FIG. 11). NanoX responds specifically to various SARS-CoV-2 RBD variants of concern and not to the RBD from MERS-CoV indicating that the sensor preserves the specificity of VHH72 (FIG. 12). This specific sensitivity of nanoX also allowed the wash-free in situ localization microscopy of its target in cells expressing SARS-CoV-2 spike protein (FIG. 13). See, e.g., Wang et al., Nat. Commun. vol. 11, 2251 (2020). The signal enhancement with nanoX is rapid, within a 500-millisecond window (FIG. 14), and ratiometric with a dynamic range of 10⁻⁷ M-10⁻⁵ M and an EC₅₀ of ˜500 nM comparable in both buffer and in human serum. Consistently, nanoX had a K_{d}˜300 nM and the limit of detection (LoD) of around 100 nM for SARS-CoV-2 RBD. Therefore, nanoX preserved the previously described EC₅₀/affinity: LoD relationship of analogous protein sensors. See, e.g., De Lorimier et al., Protein Sci. vol. 11, 2655-2675 (2002). The affinity of nanoX for SARS-CoV-2 spike protein is within the physiologically relevant range of other biosensors. See, e.g., Gulyani et al., Nature Chemical Biology 7, 437-444 (2011); and Nalbant et al., Science vol. 305, 1615-1619 (2004).

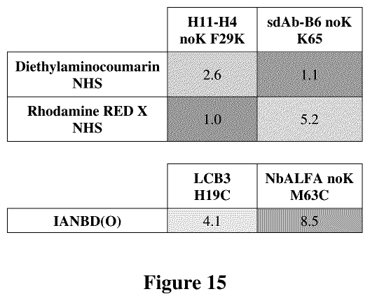

Additionally, three other SARS-CoV-2 nanosensors were engineered starting from: (1) the nanobody H11-H4 against the SARS-Cov-2 RBD; (2) the nanobody sdAb-B6 against the SARS-Cov-2 nucleocapsid protein; and (3) the mini-protein (<8 kDa) LCB3 against the SARS-Cov-2 spike protein RBD. See, e.g., Cao et al., Science vol. 370, 426-431 (2020); Huo et al., Nature structural & molecular biology vol. 27, 846-854 (2020); and Ye et al., Front. Immunol. vol. 12, 19037 (2021). H11-H4 noK F29K, sdAb-B6 noK K65, and LCB3 H19C were prepared and functionalized with the fluorogenic probes specified in FIG. 15. Fold fluorescence in the presence of spike protein is also indicated in FIG. 15.

### Example 2: Development of Nanobody Sensors of ALFA-Tag

A fluorogenic sensor was also engineered based on the nanobody NbALFA against the genetically encodable small (˜1.5 kDa) ALFA-tag. See, e.g., Götzke et al. Nature Communications vol. 10, 4403 (2019). NbALFA M63C was prepared and functionalized with IANBD. This fluorogenic sensor showed significant increase in fluorescence in the presence of the ALFA-tag peptide or targets (e.g. proteins) that are fused into the ALFA-tag peptide (FIG. 15).

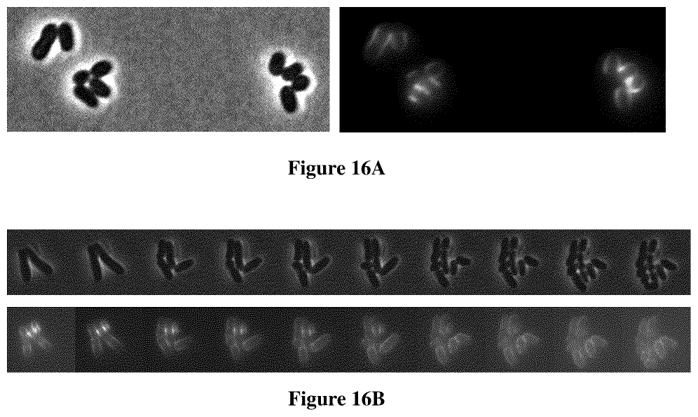

Microscopy data was collected using the ALFA-tag biosensor and a Corynebacterium glutamicum bacterium that is expressing an ALFA tagged porin protein complex on its surface. The fluorogenic ALFA-sensor allows for the labeling and imaging of the complex in a wash-free fashion (FIG. 16A). Time-lapse microscopy detected the relocalization of the complex during the cellular growth by adding the sensor into the growth medium (microscopy pads made out of Brain Heart Infusion medium) (FIG. 16B). Because the sensor is not fluorescent until it binds the porin complex, the background fluorescence is minimal and enables real-time live-cell imaging. With other imaging methods, specific staining on live-cells is generally difficult as washing away unbound fluorogenic probes can prove difficult in that setting. As demonstrated, the fluorogenic sensors provided herein enable not only faster staining, but also live-cell staining (e.g., without a washing step). This is due in part to the low background fluorescence of the unbound sensors.

### Example 3: Purification of Nanobody and Mini-Protein Sequences

For purifying unlabeled nanobodies from E. coli cytoplasms, histidine (H)-tagged open reading frame (ORF) sequences optimized for expression in E. coli were cloned into pet28a plasmids. The plasmid was cloned into BL21(DE3) Competent Cells, the protein was expressed overnight at 16-18° C. after isopropyl β-d-1-thiogalactopyranoside (IPTG) was added (˜1 mM final) to the late exponential cells.

SEQ ID NOs: 5 and 6 are particular examples of purified nanobody sequences (before conjugation of a fluorogenic probe). SEQ ID NO: 5 comprises a VHH72 wild-type sequence. SEQ ID NO: 6 is a VHH72 V104K NoK (K→R) sequence. Each sequence includes a histidine (H) tag, Thrombin cleavage sequence, and mRNA display C-terminal tag.

(SEQ ID NO: 5)
MGSSHHHHHHSSGLVPRGSHQVQLQESGGGLVQAGGSLRLSCAASGRTFS
EYAMGWFRQAPGKEREFVATISWSGGSTYYTDSVKGRFTISRDNAKNTVY
LQMNSLKPDDTAVYYCAAAGLGTVVSEWDYDYDYWGQGTQVTVSSGSGGS
GGGSGGGSG,
(SEQ ID NO: 6)
MGSSHHHHHHSSGLVPRGSHQVQLQESGGGLVQAGGSLRLSCAASGRTFS
EYAMGWFRQAPGREREFVATISWSGGSTYYTDSVRGRFTISRDNARNTVY
LQMNSLRPDDTAVYYCAAAGLGTKVSEWDYDYDYWGQGTQVTVSSGSGGS
GGGSGGGSG.

The following protocol was followed to purify the proteins from E. coli cytoplasm:
Equilibration Buffer: 20 mM Tris-HCl pH 8.3, 0.5 M NaCl, 5 mM imidazole;
Wash Buffer: 20 mM Tris-HCl pH 8.3, 0.5 M NaCl, 20 mM imidazole;
Elution Buffer: 20 mM Tris-HCl pH 8.3, 0.5 M NaCl, 200 mM imidazole.

Step 1. Pellet cells; 50 mL conical at 3500rcf for 30 mins. Pellets can be used directly or stored at −20° C. or −80° C. for long term storage or to ease lysis.

Step 2. Weigh pellet and add 4 mL per 1 g weight cell mass of BugBuster Master Mix. Swirl by hand. Incubate at RT for 45 minutes with rocking.

Step 3. Dispense 0.5 mL of His-Pur Cobalt resin to 15 mL screw cap tubes. Centrifuge at 700 g for 2 minutes. Remove buffer. Wash with 1 mL of Equilibration Buffer. Resuspend in 4 mL of Equilibration Buffer.

Step 4. Pellet lysis at 5000 g for 15 minutes and decant 4 mL of soluble fraction into slurry. Bind with rotation for 35 min at RT. Pellet resin at 700 g for 2 mins and decant soluble fraction as “flowthrough.”

Step 5. Wash 2× with 1 mL (2× resin volume). Wash Buffer by resuspending via pipette or tube inversion. Spin down at 700 g for 2 minutes.

Step 6. Elute 3× with 0.5 mL (1× resin volume). Spin at 700 g for 2 minutes between each elution.

Typically, this protocol resulted into 1-2 mg protein per 50 mL culture. Purified nanobody was dialyzed to 1× Phosphate Buffered Saline using 10 kDa Spin Columns and the nanobodies either flash frozen and stored in −80° C. or stored in +4° C.

The following are further examples of purified protein sequences (before conjugation of a fluorogenic probe):

H11-H4 nok F29K including histidine and mRNA
display tags (SEQ ID NO: 20):
(SEQ ID NO: 20)
MGSSHHHHHHSSGLVPRGSHQVQLVESGGGLMQAGGSLRLSCAVSGRTK
STAAMGWFRQAPGREREFVAAIRWSGGSAYYADSVRGRFTISRDRARNT
VYLQMNSLRYEDTAVYYCAQTHYVSYLLSDYATWPYDYWGQGTQVTVSS
GSGGSGGGSGGGSG.
sdAb-B6 noK K65 including histidine and mRNA
display tags (SEQ ID NO: 21):
(SEQ ID NO: 21)
MHHHHHHHHSSGENLYFQSSGEVQLQASGGGLVRPGGSLRLSCAASGFT
FSSYAMMWVRQAPGRGLEWVSAINGGGGSTSYADSVKGRFTISRDNARN
TLYLQMNSLRPEDTAVYYCARYQAAVHQEREDYWGQGTQVTVSSGSGGS
GGGSGGGSG.
NbALFA noK M63C including histidine and mRNA
display tags (SEQ ID NO: 22):
(SEQ ID NO: 22)
MHHHHHHHHSSGENLYFQSSGEVQLQESGGGLVQPGGSLRLSCTASGVT
ISALNAMAMGWYRQAPGERRVMVAAVSERGNACYRESVQGRFTVTRDFT
NRMVSLQMDNLRPEDTAVYYCHVLEDRVDSFHDYWGQGTQVTVSSGSGG
SGGGSGGGSG.
LCB3 H19C including histidine display tag and
thrombin cleavage sequence (SEQ ID NO: 23):
(SEQ ID NO: 23)
MGSSHHHHHHSSGLVPRGSHNDDELHMLMTDLVYEALCFAKDEEIKKRV
FQLFELADKAYKNNDRQKLEKVVEELKELLERLLS.

### Example 4: Conjugating Fluorogenic Probes to Nanobodies

For labeling nanobodies with thiol or amine reactive probes, the following procedure may be used: The nanobodies were diluted to ˜1.5 mg/mL (or ˜100 μM) final concentration. The thiol labeling was performed in the same buffer (PBS) or in presence of 50 mM Borate Buffer ˜pH 8.5 (by diluting 500 mM Borate pH 8.5 buffer into PBS). Prior reduction of the cysteines by (tris(2-carboxyethyl)phosphine) (TCEP) did not increase labeling efficiencies. The amine labeling was performed in presence of 50 mM Borate Buffer ˜pH 8.5 (by diluting 500 mM Borate pH 8.5 buffer into PBS).

Typically, reactive probes were dissolved in dry DMSO to 25 mM. The probes were diluted 100× directly to the labeling buffer, which resulted into 2-3× equivalent dye to protein ratio (at final 250 uM and 1% DMSO). The reaction was performed in room temperature, in the dark for 2 h to 4 h and quenched by the addition of excess cysteine (or glycine) to 10 mM. In general, the extend of the labeling was qualitatively determined by running labeled nanobodies on a SDS protein gel and recording the gel fluorescence on a gel doc. No quantitative Degree of Labeling (DOL) determination was done. In general, labeled nanobodies were purified using spin size exclusion columns such as Zeba™ Spin Desalting Columns, 7K MWCO. Labeled nanobodies could also be purified to uniformity using Fast Protein Liquid Chromatography. Labeled nanobodies were stored in 4° C.

### Example 5: Fluorogenic Sensing

A typical biosensing experiment involved adding different concentrations of SARS-CoV-2 spike proteins into excess labeled and purified nanobodies in final 1×PBS. The concentrations of the labeled nanobodies were not carefully controlled, but they were typically at final ˜0.1 mg/mL.

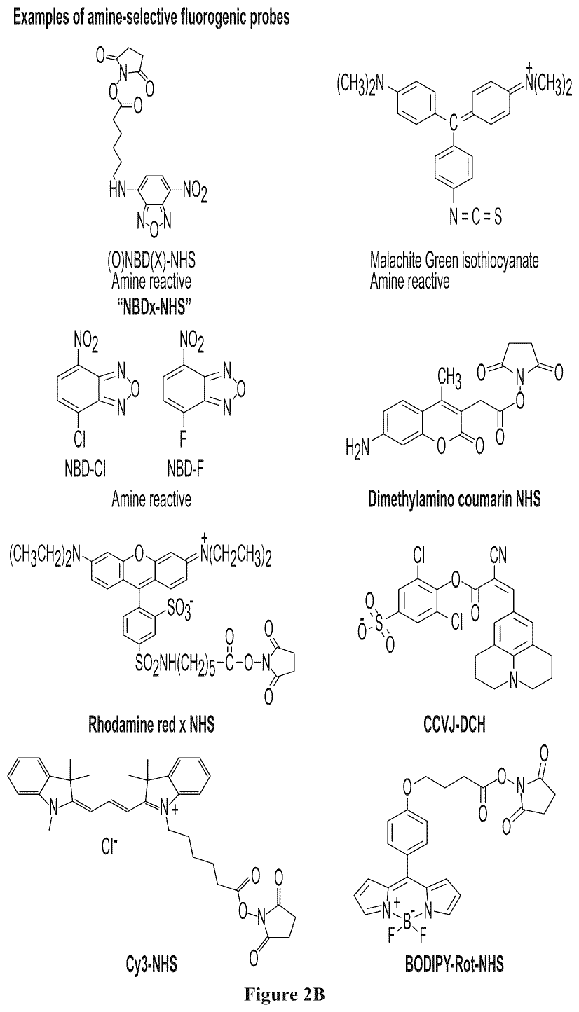

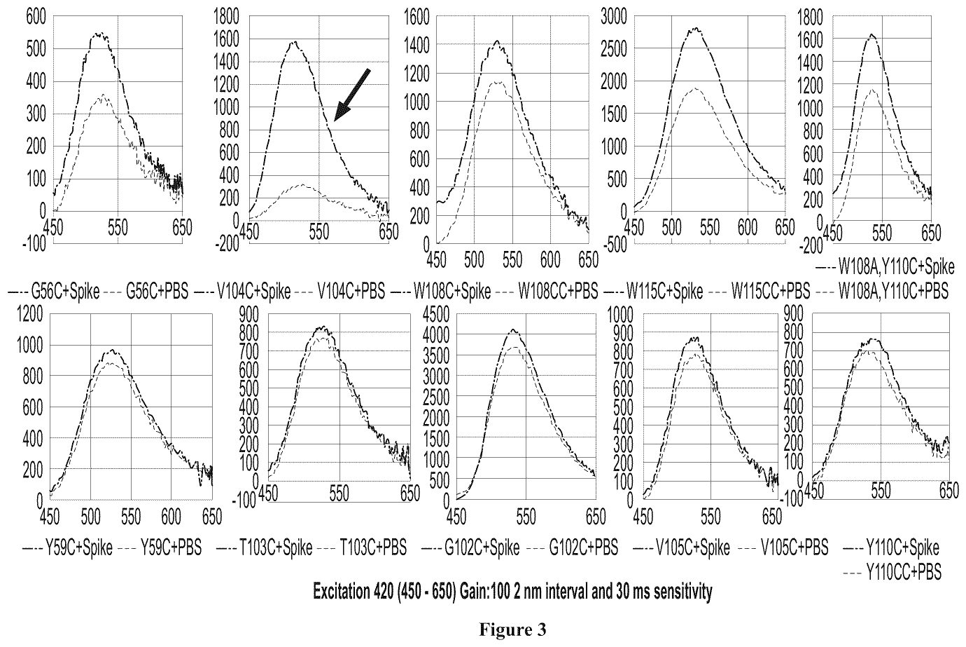

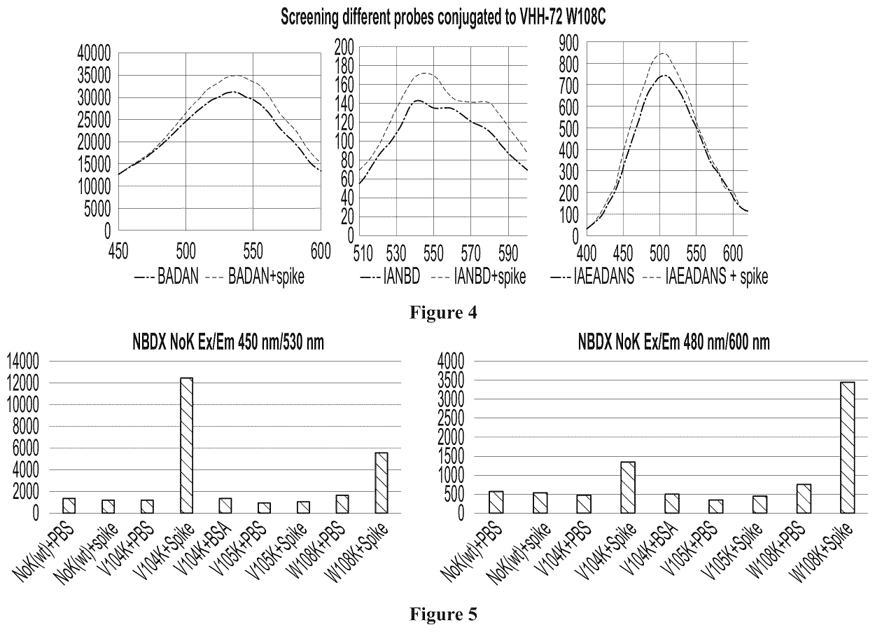

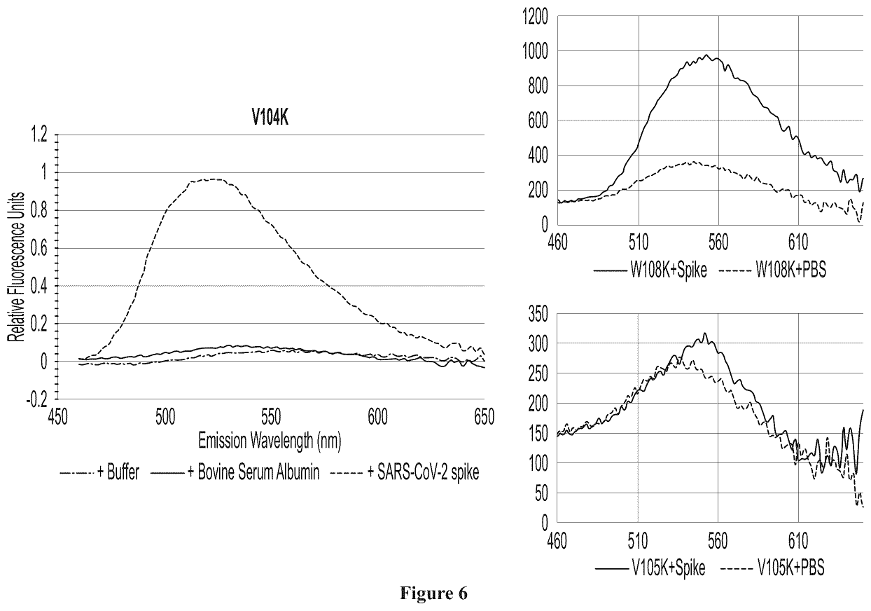

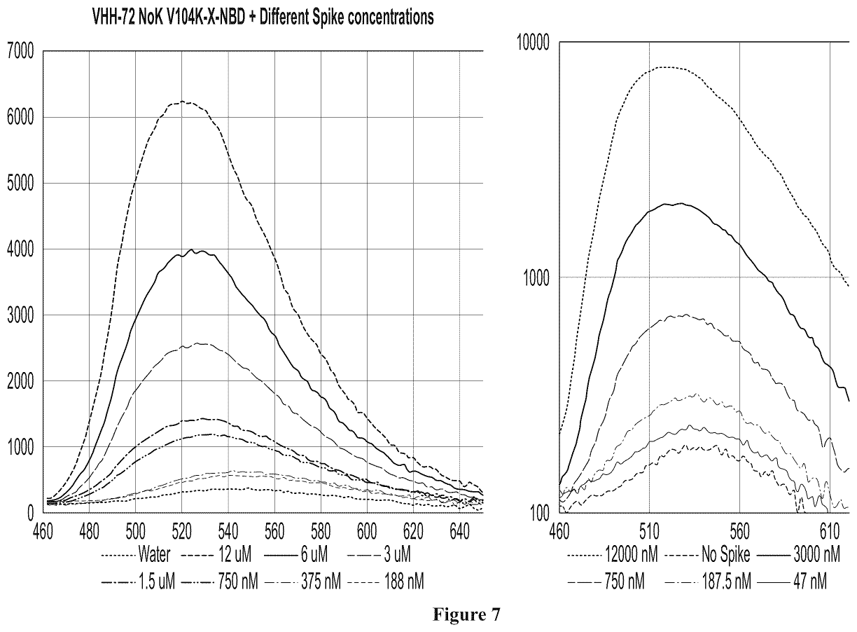

FIGS. 3-9 show the results of fluorogenic sensing experiments with different VHH72 variants conjugate to fluorogenic probes. The sensors increase in fluorescence when mixed with SARS-CoV-2 spike protein.

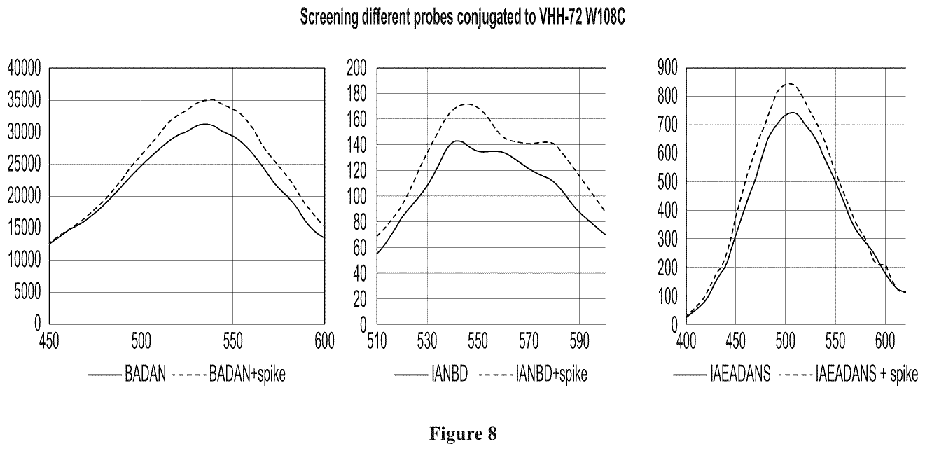

Spectra wavelength, absorbance, and fluorescence intensity of sensors in the presence of absence of the SARS-CoV-2 spike proteins were determined using a Synergy H1 plate reader (BioTek, USA). 96-well plates (measuring from ˜50 μL sample volumes Falcon Polystyrene, black walls with clear bottom) or 16-well Take3 Micro-Volume Plate (measuring from ˜2 μL sample volumes, 0.5 mm optical path length) were used for the measurements. First, the Excitation spectrum and the excitation maxima of sensors were determined by recording the absorbance with excitation light ranged from 350 to 700 nm (2 nm increment). The quantative fluorescence was measured following one of the following methods: (1) the fluorescence was measured by using fixed optimal excitation and emission wavelengths for a given fluorophore (e.g., 350 nm excitation and 450 nm Emission for Cou in FIG. 10); (2) The quantitative fluorescence spectra was determined by using a fixed excitation wavelength at ˜20 nm below the maximum absorbance and scanning emission wavelengths that covers the emission spectra of a given dye. For example the NBD-TMR FRET sensors in FIG. 9 were excited at 420 nm and the relative emission spectra was determined scanning emission intensities between 450 nm-650 nm (2 nm steps).

### Example 6: FRET-Labeled Fluorogenic Sensors

FIG. 9 outlines a FRET labeling scheme using VHH72 V104K NoK that employs thrombin cleavage and may increase the detection sensitivity of the sensors. TMR (tetramethylrhodamine) is a commercially available fluorescent probe that is not environmentally sensitive. As shown in FIG. 9, the emission spectra overlap of NBD (7-Nitrobenz-2-Oxa-1,3-Diazol-4-yl)-only labeled sensor (gray curve) and TMR-only labeled sensor (yellow curve) indicates that they are good FRET partners. In the presence of the SARS-CoV-2 spike protein, the NBD→TMR FRET significantly increases (compare red curve to the blue curve).

The general procedure above for labeling nanobodies with amine-reactive probed was used. VHH72 V104K NoK nanobody (SEQ ID NO: 6) was labeled for 2 h in room temperature, in the dark with 250 uM NBDx-NHS (FIG. 2B). The reaction was quenched by the addition of glycine to 20 mM.

The N-terminal thrombin sequence of ˜1 mg of labeled sensor was cleaved overnight at the room temperature using RECOMT THROMBIN CLEANCLEAVE KIT (Sigma). This treatment removed the N-terminal NBDx label and freed up a new unlabeled N-terminal amine (FIG. 9), represented by the following amino acid sequence (fluorogenic probe NBDx-NHS conjugated to the sole lysine (K) of the sequence):

(SEQ ID NO: 9)
GSHQVQLQESGGGLVQAGGSLRLSCAASGRTFSEYAMGWFRQAPGREREF
VATISWSGGSTYYTDSVRGRFTISRDNARNTVYLQMNSLRPDDTAVYYCA
AAGLGTKVSEWDYDYDYWGQGTQVTVSSGSGGSGGGSGGGSG.

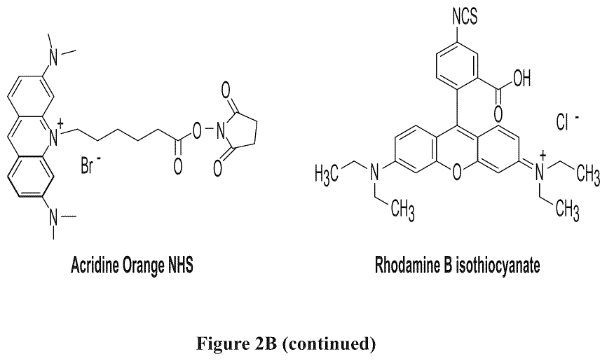

The cleaved sensor is dialyzed into PBS and concentrated using 3 kD Spin Columns. The cleaved sensor is labeled with TMR-X-NHS using the aforementioned conditions (at 250 uM, 1% DMSO final concentration in 50 mM Borate Buffer pH 8.5, 2 h, room temperature, dark) and purified using Zeba™ Spin Desalting Columns, 7K MWCO. These efforts resulted into a nanobody that is labeled with the fluorogenic (and environmentally sensitive) NBD dye at its binding interface and with an environmentally insensitive TMR dye at its N-terminus (FIG. 4). NBD emission overlaps with TMR excitation and therefore they are good FRET partners. Upon binding to SARS-CoV-2 spike protein, NBD fluorescence increases 25-100 fold, which in turn increases FRET to TMR in a binding specific manner. FRET labeling can reduce the background fluorescence of the unbound sensor increasing the sensitivity of our sensors.

### Additional Embodiments

Additional embodiments of the disclosure are represented by the following numbered paragraphs:

1. A fluorogenic sensor for detecting a target comprising:
a nanobody; and
a fluorogenic small molecule conjugated at or around a target-binding domain of the nanobody.

2. The fluorogenic sensor of paragraph 1, wherein the fluorogenic small molecule is conjugated to a target-binding domain of the nanobody.

3. The fluorogenic sensor of paragraph 1 or 2, wherein the nanobody binds an antigen.

4. The fluorogenic sensor of any one of paragraphs 1-3, wherein the nanobody binds a pathogen.

5. The fluorogenic sensor of any one of paragraphs 1-4, wherein the nanobody binds a spike protein of a coronavirus or variant thereof.

6. The fluorogenic sensor of any one of paragraphs 1-4, wherein the nanobody binds a nucleocapsid protein of a coronavirus or variant thereof.

7. The fluorogenic sensor of paragraph 5 or 6, wherein the coronavirus is a SARS-CoV-2 virus or variant thereof.

8. The fluorogenic sensor of any one of paragraphs 1-7, wherein the nanobody is a VHH72 nanobody.

9. The fluorogenic sensor of any one of paragraphs 1-8, wherein the nanobody comprises an amino acid sequence with at least 80% sequence identity with SEQ ID NO: 2.

10. The fluorogenic sensor of paragraph 9, wherein the nanobody comprises an amino acid sequence at least 85%, 90%, 91%, 92%, 93%, 94%, 95%, 96%, 97%, 98%, or 99% identical to SEQ ID NO: 2.

11. The fluorogenic sensor of paragraph 9, wherein the nanobody comprises an amino acid sequence with 100% sequence identity with SEQ ID NO: 2.

12. The fluorogenic sensor of any one of paragraphs 9-11, wherein the target-binding domain is from amino acids 26-35, 50-59, or 99-114 of the amino acid sequence.

13. The fluorogenic sensor of paragraph 12, wherein the target-binding domain is from amino acids 99-114 of the amino acid sequence.

14. The fluorogenic sensor of any one of paragraphs 1-13, wherein the fluorogenic small molecule is conjugated to a lysine or cysteine residue of nanobody.

15. The fluorogenic sensor of any one of paragraphs 1-14, wherein the nanobody sequence comprises one or more amino acids substituted by lysine (K) or cysteine (C), wherein the fluorogenic small molecule is conjugated to one of said lysine or cysteine.

16. The fluorogenic sensor of any one of paragraphs 9-15, wherein amino acid sequence comprises at least one amino acid substitution selected from I51C, W53C, G56C, Y59C, G102C, T103C, V104C, V105C, W108C, Y110C, and W115C; and wherein the fluorogenic small molecule is conjugated to the cysteine at the substituted position.

17. The fluorogenic sensor of any one of paragraphs 9-15, wherein the amino acid sequence comprises at least one amino acid substitution selected from W53K, V104K, V105K, W108K, Y110K, and W115K; and wherein the fluorogenic small molecule is conjugated to the lysine at the substituted position.

18. The fluorogenic sensor of any one of paragraphs 1-17, wherein one or more other lysines of the nanobody, other than the lysine conjugated to the fluorogenic small molecule, are independently substituted by a different amino acid.

19. The fluorogenic sensor of paragraph 18, wherein one or more other lysines of the nanobody, other than the lysine conjugated to the fluorogenic small molecule, are independently substituted by arginine (R).

20. The fluorogenic sensor of any one of paragraphs 1-19, wherein the nanobody comprises an amino acid sequence with at least 80% sequence identity with SEQ ID NO: 4.

21. The fluorogenic sensor of paragraph 20, wherein the nanobody comprises an amino acid sequence with about 90%, 91%, 92%, 93%, 94%, 95%, 96%, 97%, 98%, or 99% sequence identity with SEQ ID NO: 4.

22. The fluorogenic sensor of paragraph 20, wherein the nanobody comprises an amino acid sequence with 100% sequence identity with SEQ ID NO: 4.

23. The fluorogenic sensor of paragraph 22, wherein the fluorogenic sensor is VHH72 noK V104NBDxK.

24. The fluorogenic sensor of any one of paragraphs 1-7, wherein the nanobody is a H11-H4 nanobody.

25. The fluorogenic sensor of any one of paragraphs 1-7 and 24, wherein the nanobody comprises an amino acid sequence with at least 80% sequence identity with SEQ ID NO: 10.

26. The fluorogenic sensor of paragraph 25, wherein the nanobody comprises an amino acid sequence at least 85%, 90%, 91%, 92%, 93%, 94%, 95%, 96%, 97%, 98%, 99%, or 100% identical to SEQ ID NO: 10.

27. The fluorogenic sensor of any one of paragraphs 24-26, wherein the nanobody comprises one or more amino acids substituted by lysine (K), wherein the fluorogenic small molecule is conjugated to one of said lysine.

28. The fluorogenic sensor of any one of paragraphs 25-27, wherein amino acid sequence comprises a F29K substitution; and wherein the fluorogenic small molecule is conjugated to the lysine at the substituted position.

29. The fluorogenic sensor of any one of paragraphs 24-28, wherein one or more other lysines of the nanobody, other than the lysine conjugated to the fluorogenic small molecule, are independently substituted by a different amino acid.

30. The fluorogenic sensor of paragraph 29, wherein one or more other lysines of the nanobody, other than the lysine conjugated to the fluorogenic small molecule, are independently substituted by arginine (R).

31. The fluorogenic sensor of any one of paragraphs 1-7 and 24-30, wherein the nanobody comprises an amino acid sequence with at least 80% sequence identity with SEQ ID NO: 11.

32. The fluorogenic sensor of paragraph 31, wherein the nanobody comprises an amino acid sequence with about 90%, 91%, 92%, 93%, 94%, 95%, 96%, 97%, 98%, 99% or 100% sequence identity with SEQ ID NO: 11.

33. The fluorogenic sensor of paragraph 32, wherein the nanobody is H11-H4 noK F29K.

34. The fluorogenic sensor of any one of paragraphs 1-7, wherein the nanobody is a sdAb-B6 nanobody.

35. The fluorogenic sensor of any one of paragraphs 1-7 and 34, wherein the nanobody comprises an amino acid sequence with at least 80% sequence identity with SEQ ID NO: 12.

36. The fluorogenic sensor of paragraph 35, wherein the nanobody comprises an amino acid sequence at least 85%, 90%, 91%, 92%, 93%, 94%, 95%, 96%, 97%, 98%, 99%, or 100% identical to SEQ ID NO: 12.

37. The fluorogenic sensor of any one of paragraphs 34-36, wherein the nanobody comprises one or more lysine (K) residues, wherein the fluorogenic small molecule is conjugated to one of said lysine.

38. The fluorogenic sensor of paragraph 37, wherein amino acid sequence comprises K65; and wherein the fluorogenic small molecule is conjugated to the lysine at that position.

39. The fluorogenic sensor of any one of paragraphs 34-38, wherein one or more other lysines of the nanobody, other than the lysine conjugated to the fluorogenic small molecule, are independently substituted by a different amino acid.

40. The fluorogenic sensor of paragraph 39, wherein one or more other lysines of the nanobody, other than the lysine conjugated to the fluorogenic small molecule, are independently substituted by arginine (R).

41. The fluorogenic sensor of any one of paragraphs 1-7 and 34-40, wherein the nanobody comprises an amino acid sequence with at least 80% sequence identity with SEQ ID NO: 13.

42. The fluorogenic sensor of paragraph 41, wherein the nanobody comprises an amino acid sequence with about 90%, 91%, 92%, 93%, 94%, 95%, 96%, 97%, 98%, 99% or 100% sequence identity with SEQ ID NO: 13.

43. The fluorogenic sensor of paragraph 42, wherein the nanobody is sdAb-B6 noK K65.

44. The fluorogenic sensor of paragraph 1 or 2, wherein the nanobody binds an epitope tag.

45. The fluorogenic sensor of any one of paragraphs 1, 2, and 44, wherein the nanobody binds ALFA-tag.

46. The fluorogenic sensor of any one of paragraphs 1, 2, 44, and 45, wherein the nanobody is a NbALFA nanobody.

47. The fluorogenic sensor of any one of paragraphs 1, 2, and 44-46, wherein the nanobody comprises an amino acid sequence with at least 80% sequence identity with SEQ ID NO: 16.

48. The fluorogenic sensor of paragraph 47, wherein the nanobody comprises an amino acid sequence at least 85%, 90%, 91%, 92%, 93%, 94%, 95%, 96%, 97%, 98%, 99%, or 100% identical to SEQ ID NO: 16.

49. The fluorogenic sensor of any one of paragraphs 44-48, wherein the nanobody comprises one or more amino acids substituted by cysteine (C), wherein the fluorogenic small molecule is conjugated to one of said cysteine.

50. The fluorogenic sensor of any one of paragraphs 47-49, wherein amino acid sequence comprises a M63C substitution; and wherein the fluorogenic small molecule is conjugated to the cysteine at the substituted position.

51. The fluorogenic sensor of any one of paragraphs 1, 2, and 44-50, wherein the nanobody comprises an amino acid sequence with at least 80% sequence identity with SEQ ID NO: 17.

52. The fluorogenic sensor of paragraph 51, wherein the nanobody comprises an amino acid sequence with about 90%, 91%, 92%, 93%, 94%, 95%, 96%, 97%, 98%, 99% or 100% sequence identity with SEQ ID NO: 17.

53. The fluorogenic sensor of paragraph 52, wherein the nanobody is NbALFA M63C.

54. The fluorogenic sensor of paragraph 1 or 2, wherein the nanobody binds a small molecule.

55. The fluorogenic sensor of any one of paragraphs 1, 2, and 54 wherein the nanobody binds cortisol.

56. The fluorogenic sensor of any one of paragraphs 1, 2, 54, and 55, wherein the nanobody is a NbCor nanobody.

57. The fluorogenic sensor of any one of paragraphs 1, 2, and 54-56, wherein the nanobody comprises an amino acid sequence with at least 80% sequence identity with SEQ ID NO: 18.

58. The fluorogenic sensor of paragraph 57, wherein the nanobody comprises an amino acid sequence at least 85%, 90%, 91%, 92%, 93%, 94%, 95%, 96%, 97%, 98%, or 99% identical to SEQ ID NO: 18.

59. The fluorogenic sensor of any one of paragraphs 54-58, wherein the fluorogenic small molecule is conjugated to a lysine or cysteine residue of nanobody.

60. The fluorogenic sensor of any one of paragraphs 54-59, wherein the nanobody comprises one or more amino acids substituted by lysine (K) or cysteine (C), wherein the fluorogenic small molecule is conjugated to one of said lysine or cysteine.

61. The fluorogenic sensor of any one of paragraphs 57-60, wherein amino acid sequence comprises at least one amino acid substitution selected from T53C, N27C, T28C, S30C, V24C, G29C, and W34C; and wherein the fluorogenic small molecule is conjugated to the cysteine at the substituted position.

62. The fluorogenic sensor of any one of paragraphs 57-60, wherein the amino acid sequence comprises at least one amino acid substitution selected from V24K and A78K; and wherein the fluorogenic small molecule is conjugated to the lysine at the substituted position.

63. The fluorogenic sensor of any one of paragraphs 57-59, wherein the fluorogenic small molecule is conjugated to the lysine at position K79.

64. The fluorogenic sensor of any one of paragraphs 54-63, wherein one or more other lysines of the nanobody, other than the lysine conjugated to the fluorogenic small molecule, are independently substituted by a different amino acid.

65. The fluorogenic sensor of paragraph 64, wherein one or more other lysines of the nanobody, other than the lysine conjugated to the fluorogenic small molecule, are independently substituted by arginine (R).

66. The fluorogenic sensor of any one of paragraphs 1, 2, and 54-65, wherein the nanobody comprises an amino acid sequence with at least 80% sequence identity with SEQ ID NO: 19.

67. The fluorogenic sensor of paragraph 66, wherein the nanobody comprises an amino acid sequence with about 90%, 91%, 92%, 93%, 94%, 95%, 96%, 97%, 98%, 99%, or 100% sequence identity with SEQ ID NO: 19.

68. The fluorogenic sensor of paragraph 67, wherein the nanobody is NbCor T53C.

69. A fluorogenic sensor for detecting a target comprising: a mini-protein; and a fluorogenic small molecule conjugated to the mini-protein.

70. The fluorogenic sensor of paragraph 69, wherein the fluorogenic small molecule is conjugated at or around a target-binding domain of the mini-protein.

71. The fluorogenic sensor of paragraph 70, wherein the fluorogenic small molecule is conjugated to a target-binding domain of the mini-protein.

72. The fluorogenic sensor of any one of paragraphs 69-71, wherein the mini-protein binds an antigen.

73. The fluorogenic sensor of any one of paragraphs 69-72, wherein the mini-protein binds a pathogen.

74. The fluorogenic sensor of any one of paragraphs 69-73, wherein the mini-protein binds a spike protein of a coronavirus or variant thereof.

75. The fluorogenic sensor of paragraph 74, wherein the coronavirus is a SARS-CoV-2 virus or variant thereof.

76. The fluorogenic sensor of any one of paragraph 69-75, wherein the mini-protein is a LCB3 protein.

77. The fluorogenic sensor of any one of paragraphs 69-76, wherein the mini-protein comprises an amino acid sequence with at least 80% sequence identity with SEQ ID NO: 14.

78. The fluorogenic sensor of paragraph 77, wherein the mini-protein comprises an amino acid sequence at least 85%, 90%, 91%, 92%, 93%, 94%, 95%, 96%, 97%, 98%, 99%, or 100% identical to SEQ ID NO: 14.

79. The fluorogenic sensor of any one of paragraphs 69-78, wherein the mini-protein comprises one or more amino acids substituted by cysteine (C), wherein the fluorogenic small molecule is conjugated to one of said cysteine.

80. The fluorogenic sensor of any one of paragraphs 77-79, wherein amino acid sequence comprises a H19C substitution; and wherein the fluorogenic small molecule is conjugated to the cysteine at the substituted position.

81. The fluorogenic sensor of any one of paragraphs 69-80, wherein the mini-protein comprises an amino acid sequence with at least 80% sequence identity with SEQ ID NO: 15.

82. The fluorogenic sensor of paragraph 81, wherein the mini-protein comprises an amino acid sequence with about 90%, 91%, 92%, 93%, 94%, 95%, 96%, 97%, 98%, 99% or 100% sequence identity with SEQ ID NO: 15.

83. The fluorogenic sensor of paragraph 82, wherein the mini-protein is LCB3 H19C.

84. The fluorogenic sensor of any one of paragraphs 1-83, wherein the fluorogenic small molecule conjugated to the nanobody or the mini-protein results from conjugating a compound of the following formula: FG-L-A, or a salt, stereoisomer, or tautomer thereof; wherein FG is the fluorogenic small molecule; L is a bond or a linker; and A is a reactive moiety.

85. The fluorogenic sensor of paragraph 84, wherein A is a lysine- or cysteine-selective reactive moiety.

86. The fluorogenic sensor of paragraph 84 or 85, wherein FG is of one of the following formulae:

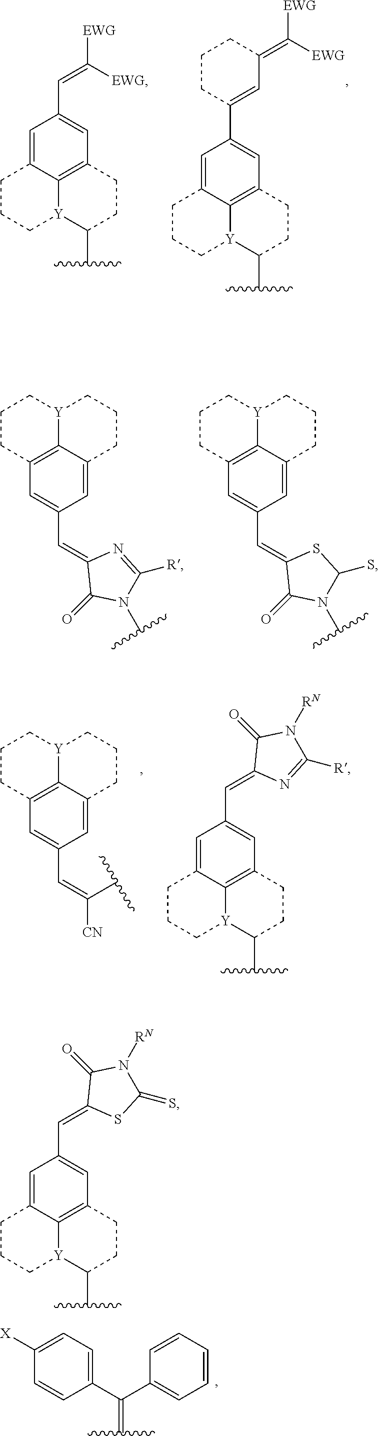

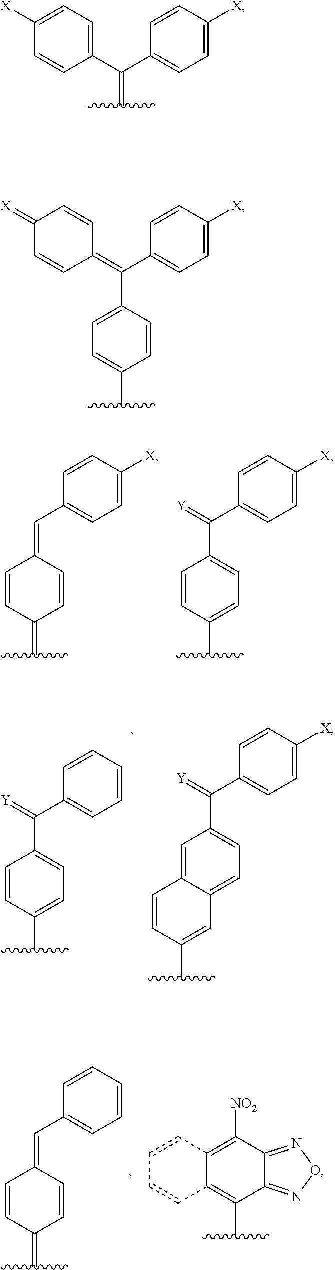

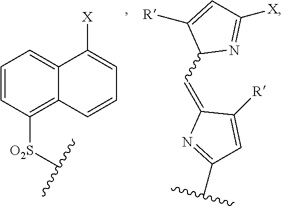

or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

87. The fluorogenic sensor of any one of paragraphs 84-86, wherein -L-A is of one of the following formulae:

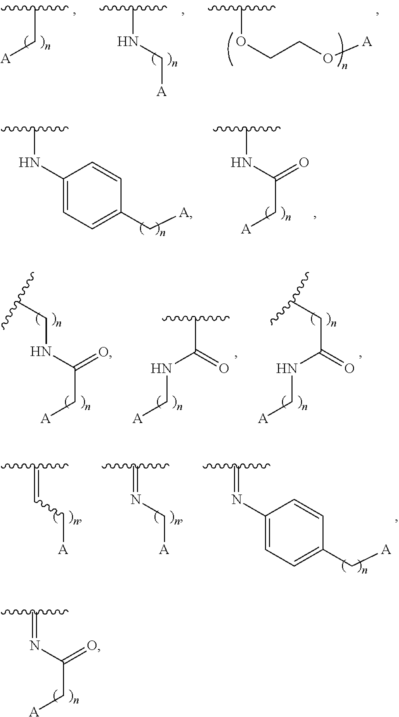

or a salt, stereoisomer, or tautomer thereof; wherein:
each n is independently 0 or an integer from 1-20, inclusive; and
wherein each formula is further optionally substituted.

88. The fluorogenic sensor of any one of paragraphs 84-87, wherein A comprises a halogen, alkene, alkyne, azide, tetrazine, or a moiety of one of the following formulae:

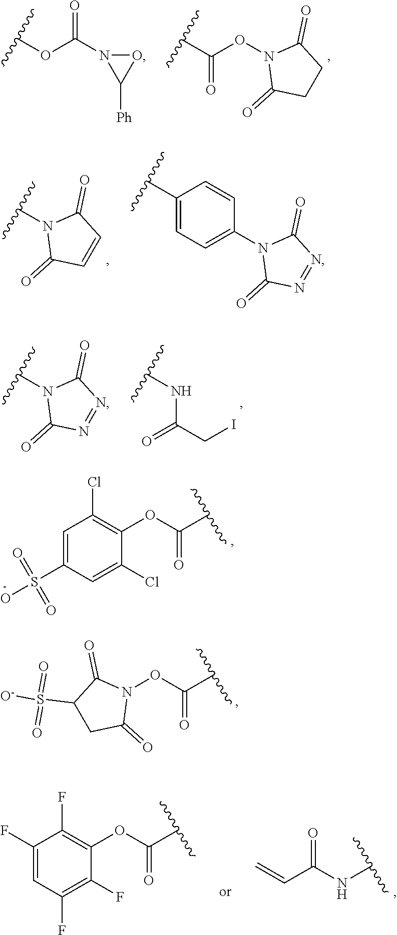

or a salt, stereoisomer, or tautomer thereof; wherein each formula is further optionally substituted.

89. The fluorogenic sensor of any one of the preceding paragraphs, wherein the the nanobody or the mini-protein comprises an unnatural amino acid comprising the fluorogenic small molecule; optionally wherein a target-binding domain of the nanobody or the mini-protein comprises an unnatural amino acid comprising the fluorogenic small molecule.

90. The fluorogenic sensor of any one of the preceding paragraphs, wherein the amino acid sequence comprises at least one amino acid substituted by an unnatural amino acid comprising the fluorogenic small molecule.

91. The fluorogenic sensor of paragraph 89 or 90, wherein the unnatural amino acid comprising the fluorogenic small molecule is encoded into the amino acid sequence or installed via transpeptidation.

92. The fluorogenic sensor of paragraph 89 or 90, wherein the unnatural amino acid comprising the fluorogenic small molecule is encoded into the amino acid sequence via ribosomal synthesis.

93. The fluorogenic sensor of any one of paragraphs 89-92, wherein the unnatural amino acid comprising the fluorogenic small molecule is of one of the following formulae:

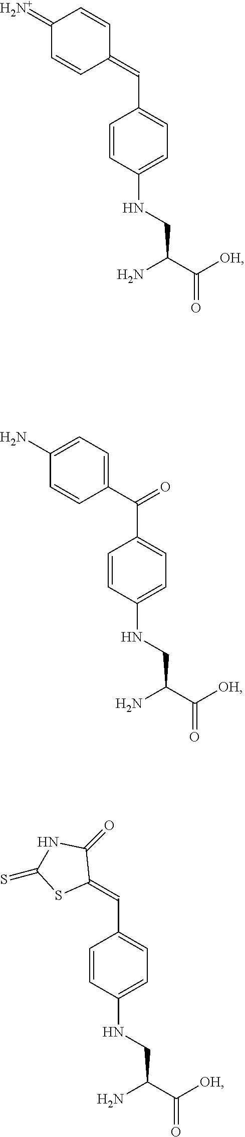 [SMILES: N[C@@H](CNc1ccc(C=C2C=CC(=[NH2+])C=C2)cc1)C(=O)O ; Nc1ccc(C(=O)c2ccc(NC[C@H](N)C(=O)O)cc2)cc1 ; N[C@@H](CNc1ccc(/C=C2\SC(=S)NC2=O)cc1)C(=O)O]

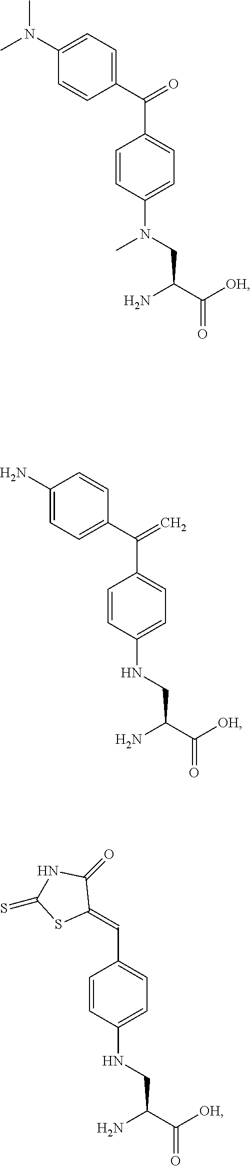 [SMILES: CN(C)c1ccc(C(=O)c2ccc(N(C)C[C@H](N)C(=O)O)cc2)cc1 ; C=C(c1ccc(N)cc1)c1ccc(NC[C@H](N)C(=O)O)cc1 ; N[C@@H](CNc1ccc(/C=C2\SC(=S)NC2=O)cc1)C(=O)O]

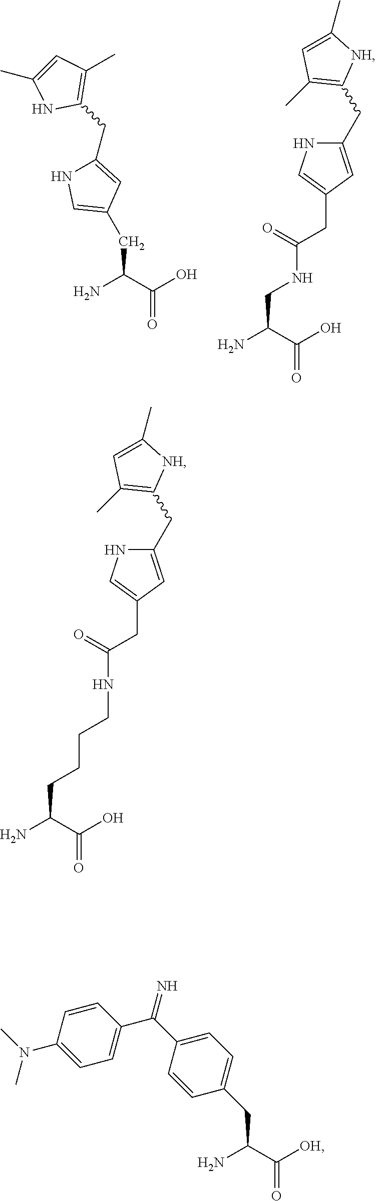 [SMILES: Cc1cc(C)c(Cc2cc(C[C@H](N)C(=O)O)c[nH]2)[nH]1 ; Cc1cc(C)c(Cc2cc(CC(=O)NC[C@H](N)C(=O)O)c[nH]2)[nH]1 ; Cc1cc(C)c(Cc2cc(CC(=O)NCCCC[C@H](N)C(=O)O)c[nH]2)[nH]1 ; CN(C)c1ccc(C(=N)c2ccc(C[C@H](N)C(=O)O)cc2)cc1]

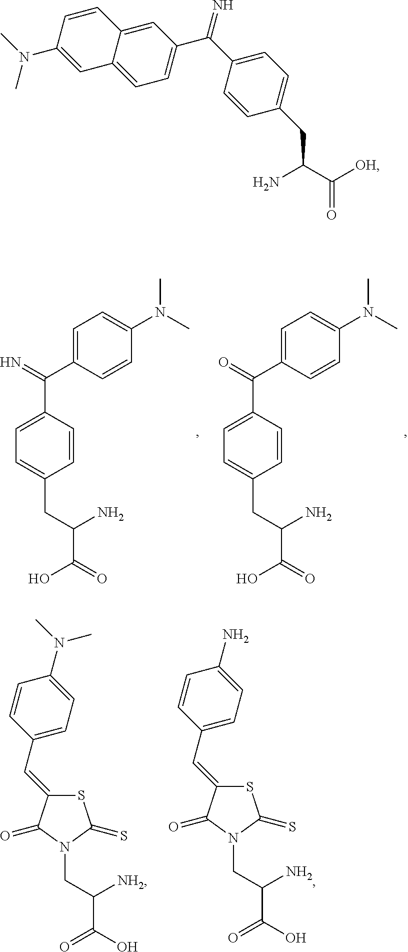

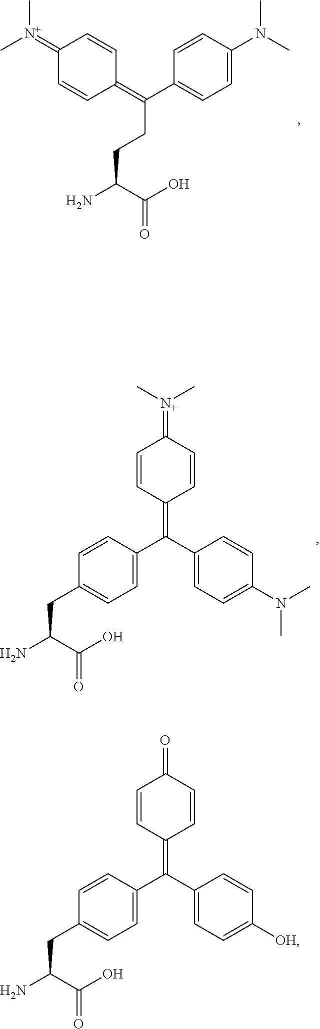

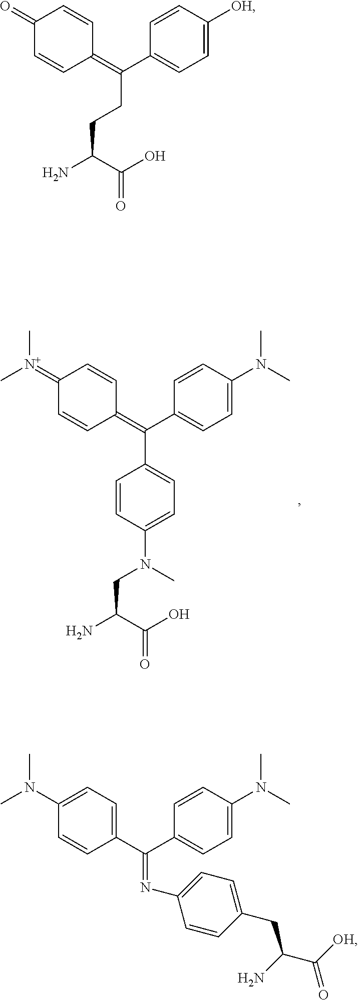

or a salt, stereoisomer, or tautomer thereof.

94. The fluorogenic sensor of any one of the preceding paragraphs, wherein the fluorogenic small molecule is 7-nitrobenz-2-Oxa-1,3-Diazol-4-yl (NBD).

95. The fluorogenic sensor of any one of the preceding paragraphs, wherein the fluorogenic small molecule conjugated to the nanobody or the mini-protein results from conjugating a compound selected from those in FIG. 2A and FIG. 2B.

96. The fluorogenic sensor of any one of the preceding paragraphs, wherein the fluorescence of the fluorogenic small molecule increases upon binding of the nanobody or the mini-protein to the target.

97. The fluorogenic sensor of any one of the preceding paragraphs, wherein the fluorescence lifetime of the fluorogenic small molecule changes upon binding of the nanobody or the mini-protein to the target.

98. The fluorogenic sensor of any one of the preceding paragraphs further comprising a second fluorogenic small molecule conjugated to the N-terminus of the nanobody.

99. The fluorogenic sensor of paragraph 98, wherein the first fluorogenic small molecule and the second fluorogenic small molecule are a fluorescence resonance energy transfer (FRET) pair.

100. The fluorogenic sensor of 98 or 99, wherein the second fluorogenic small molecule is tetramethylrhodamine (TMR).

101. A method of detecting a target, the method comprising:
(i) contacting a target with a fluorogenic sensor of any one of the preceding paragraphs; and
(ii.a) measuring or observing the fluorescence of the fluorogenic sensor, or (ii.b) measuring or observing a change in the fluorescence lifetime of the fluorogenic sensor.

102. A method of detecting an antigen, the method comprising:
(i) contacting an antigen with a fluorogenic sensor of any one of the preceding paragraphs; and
(ii.a) measuring or observing the fluorescence of the fluorogenic sensor, or (ii.b) measuring or observing a change in the fluorescence lifetime of the fluorogenic sensor.

103. The method of paragraph 102, wherein the antigen is pathogen.

104. The method of paragraphs 103, wherein the pathogen is a coronavirus or variant thereof.

105. The method of paragraph 104, wherein the coronavirus is SARS-CoV-2 or variant thereof.

106. The method of paragraph 101, wherein the target is an ALFA-tagged protein.

107. The method of paragraph 106, wherein the target is a bacterial cell expressing an ALFA-tagged protein.

108. The method of paragraph 101, wherein the target is cortisol.

109. The method of any one of paragraphs 101-108, wherein a change in fluorescence and/or fluorescence lifetime is observed instantaneously after the contacting step.

110. The method of paragraph 109, wherein the change in fluorescence and/or fluorescence lifetime is observed within less than 1 second after the contacting step.

111. The method of paragraph 110, wherein change in fluorescence and/or fluorescence lifetime is observed within less than less than 2500, 2000, 1500, 1000, 750, 500, or 250 milliseconds (ms) after the contacting step.

112. The method of any one of paragraphs 101-111, wherein an increase in fluorescence of at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold is observed.

113. A compound of the following formula:

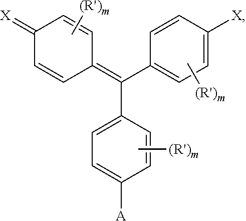

or a salt, stereoisomer, or tautomer thereof, wherein:
A is a reactive moiety comprising one of the following formulae:

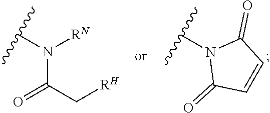

R^{H} is halogen or a leaving group;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

114. The compound of paragraph 113, wherein the compound is selected from the group consisting of:

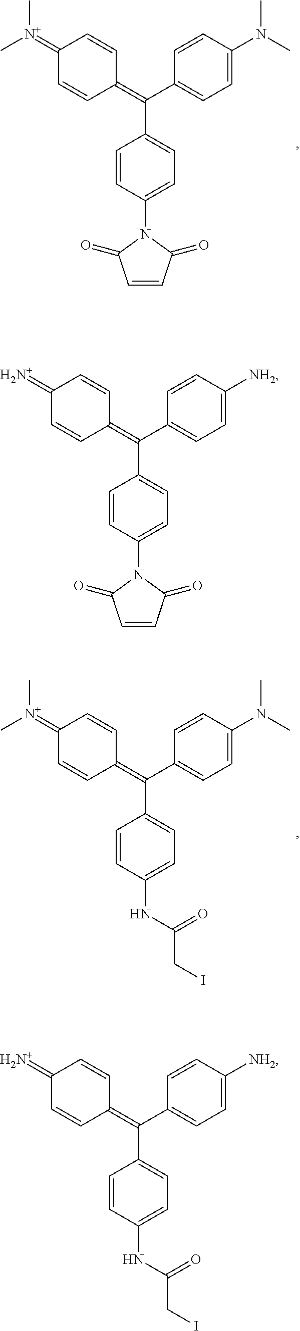

and salts, stereoisomers, and tautomers thereof.

115. A compound of the following formula:

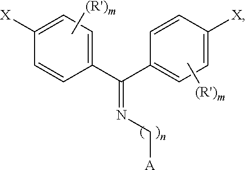

or a salt or tautomer thereof, wherein:
A is a reactive moiety;
each instance of X is independently —N(R^{N})₂, —OR^{O}, or —SR^{S};
n is 0 or an integer from 1-20, inclusive.
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4;
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

116. The compound of paragraph 115, wherein A comprises a halogen, alkyne, azide, or a moiety of one of the following formulae:

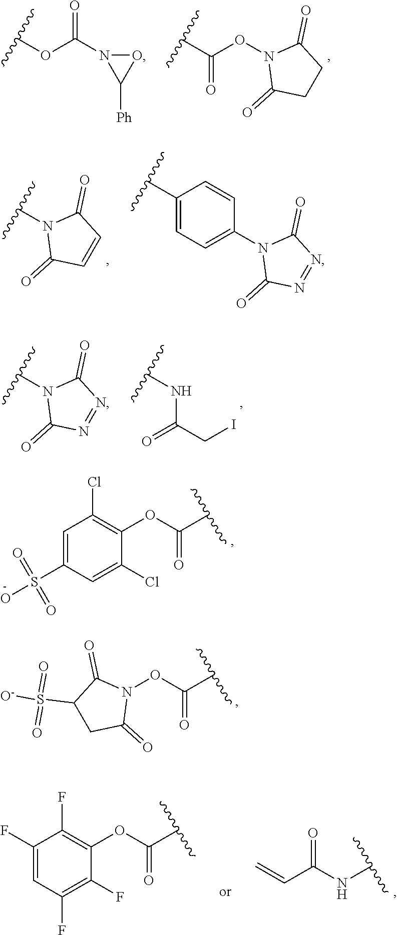

or a salt, stereoisomer, or tautomer thereof; wherein each formula is further optionally substituted.

117. The compound of paragraph 116, wherein the compound is of the formula:

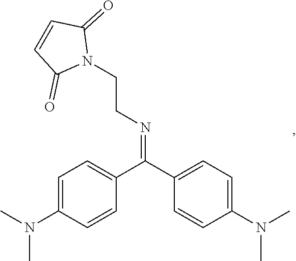

or a salt or tautomer thereof.

118. A compound of the following formula:

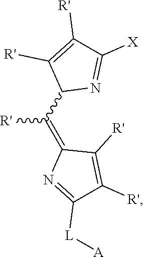

or a salt, stereoisomer, or tautomer thereof; wherein:
L is a bond or a linker;
A is a reactive moiety;
each instance of R′ and X is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

119. The compound of paragraph 118, wherein the compound is of the following formula:

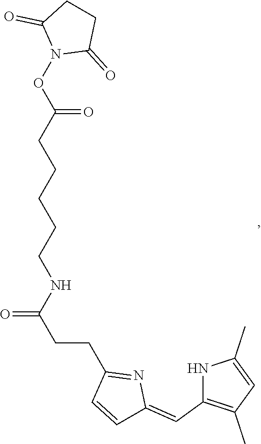 [SMILES: Cc1cc(C)c(/C=C2/C=CC(CCC(=O)NCCCCCC(=O)ON3C(=O)CCC3=O)=N2)[nH]1]

or a salt or tautomer thereof.

120. A compound of the following formula:

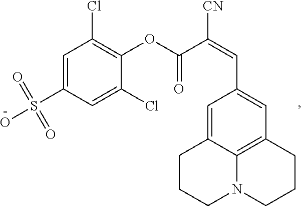 [SMILES: N#C/C(=C/c1cc2c3c(c1)CCCN3CCC2)C(=O)Oc1c(Cl)cc(S(=O)(=O)O)cc1Cl]

or a salt, stereoisomer, or tautomer thereof; wherein the compound is further optionally substituted.

121. A kit comprising a fluorogenic sensor or compound of any one of the preceding paragraphs, and optionally instructions for use.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A fluorogenic sensor for detecting a target comprising:
a nanobody; and
a fluorogenic small molecule conjugated at or around a target-binding domain of the nanobody.

2. The fluorogenic sensor of claim 1, wherein the fluorogenic small molecule is conjugated to a target-binding domain of the nanobody.

3. The fluorogenic sensor of claim 1 or 2, wherein the nanobody binds an antigen.

4. The fluorogenic sensor of any one of claims 1-3, wherein the nanobody binds a pathogen.

5. The fluorogenic sensor of any one of claims 1-4, wherein the nanobody binds a spike protein of a coronavirus or variant thereof.

6. The fluorogenic sensor of any one of claims 1-4, wherein the nanobody binds a nucleocapsid protein of a coronavirus or variant thereof.

7. The fluorogenic sensor of claim 5 or 6, wherein the coronavirus is a SARS-CoV-2 virus or variant thereof.

8. The fluorogenic sensor of any one of claims 1-7, wherein the nanobody is a VHH72 nanobody.

9. The fluorogenic sensor of any one of claims 1-8, wherein the nanobody comprises an amino acid sequence with at least 80% sequence identity with SEQ ID NO: 2.

10. The fluorogenic sensor of claim 9, wherein the nanobody comprises an amino acid sequence at least 85%, 90%, 91%, 92%, 93%, 94%, 95%, 96%, 97%, 98%, or 99% identical to SEQ ID NO: 2.

11. The fluorogenic sensor of claim 9, wherein the nanobody comprises an amino acid sequence with 100% sequence identity with SEQ ID NO: 2.

12. The fluorogenic sensor of any one of claims 9-11, wherein the target-binding domain is from amino acids 26-35, 50-59, or 99-114 of the amino acid sequence.

13. The fluorogenic sensor of claim 12, wherein the target-binding domain is from amino acids 99-114 of the amino acid sequence.

14. The fluorogenic sensor of any one of claims 1-13, wherein the fluorogenic small molecule is conjugated to a lysine or cysteine residue of nanobody.

15. The fluorogenic sensor of any one of claims 1-14, wherein the nanobody sequence comprises one or more amino acids substituted by lysine (K) or cysteine (C), wherein the fluorogenic small molecule is conjugated to one of said lysine or cysteine.

16. The fluorogenic sensor of any one of claims 9-15, wherein amino acid sequence comprises at least one amino acid substitution selected from I51C, W53C, G56C, Y59C, G102C, T103C, V104C, V105C, W108C, Y110C, and W115C; and wherein the fluorogenic small molecule is conjugated to the cysteine at the substituted position.

17. The fluorogenic sensor of any one of claims 9-15, wherein the amino acid sequence comprises at least one amino acid substitution selected from W53K, V104K, V105K, W108K, Y110K, and W115K; and wherein the fluorogenic small molecule is conjugated to the lysine at the substituted position.

18. The fluorogenic sensor of any one of claims 1-17, wherein one or more other lysines of the nanobody, other than the lysine conjugated to the fluorogenic small molecule, are independently substituted by a different amino acid.

19. The fluorogenic sensor of claim 18, wherein one or more other lysines of the nanobody, other than the lysine conjugated to the fluorogenic small molecule, are independently substituted by arginine (R).

20. The fluorogenic sensor of any one of claims 1-19, wherein the nanobody comprises an amino acid sequence with at least 80% sequence identity with SEQ ID NO: 4.

21. The fluorogenic sensor of claim 20, wherein the nanobody comprises an amino acid sequence with about 90%, 91%, 92%, 93%, 94%, 95%, 96%, 97%, 98%, or 99% sequence identity with SEQ ID NO: 4.

22. The fluorogenic sensor of claim 20, wherein the nanobody comprises an amino acid sequence with 100% sequence identity with SEQ ID NO: 4.

23. The fluorogenic sensor of claim 22, wherein the fluorogenic sensor is VHH72 noK V104NBDxK.

24. The fluorogenic sensor of any one of claims 1-7, wherein the nanobody is a H11-H4 nanobody.

25. The fluorogenic sensor of any one of claims 1-7 and 24, wherein the nanobody comprises an amino acid sequence with at least 80% sequence identity with SEQ ID NO: 10.

26. The fluorogenic sensor of claim 25, wherein the nanobody comprises an amino acid sequence at least 85%, 90%, 91%, 92%, 93%, 94%, 95%, 96%, 97%, 98%, 99%, or 100% identical to SEQ ID NO: 10.

27. The fluorogenic sensor of any one of claims 24-26, wherein the nanobody comprises one or more amino acids substituted by lysine (K), wherein the fluorogenic small molecule is conjugated to one of said lysine.

28. The fluorogenic sensor of any one of claims 25-27, wherein amino acid sequence comprises a F29K substitution; and wherein the fluorogenic small molecule is conjugated to the lysine at the substituted position.

29. The fluorogenic sensor of any one of claims 24-28, wherein one or more other lysines of the nanobody, other than the lysine conjugated to the fluorogenic small molecule, are independently substituted by a different amino acid.

30. The fluorogenic sensor of claim 29, wherein one or more other lysines of the nanobody, other than the lysine conjugated to the fluorogenic small molecule, are independently substituted by arginine (R).

31. The fluorogenic sensor of any one of claims 1-7 and 24-30, wherein the nanobody comprises an amino acid sequence with at least 80% sequence identity with SEQ ID NO: 11.

32. The fluorogenic sensor of claim 31, wherein the nanobody comprises an amino acid sequence with about 90%, 91%, 92%, 93%, 94%, 95%, 96%, 97%, 98%, 99% or 100% sequence identity with SEQ ID NO: 11.

33. The fluorogenic sensor of claim 32, wherein the nanobody is H11-H4 noK F29K.

34. The fluorogenic sensor of any one of claims 1-7, wherein the nanobody is a sdAb-B6 nanobody.

35. The fluorogenic sensor of any one of claims 1-7 and 34, wherein the nanobody comprises an amino acid sequence with at least 80% sequence identity with SEQ ID NO: 12.

36. The fluorogenic sensor of claim 35, wherein the nanobody comprises an amino acid sequence at least 85%, 90%, 91%, 92%, 93%, 94%, 95%, 96%, 97%, 98%, 99%, or 100% identical to SEQ ID NO: 12.

37. The fluorogenic sensor of any one of claims 34-36, wherein the nanobody comprises one or more lysine (K) residues, wherein the fluorogenic small molecule is conjugated to one of said lysine.

38. The fluorogenic sensor of claim 37, wherein amino acid sequence comprises K65; and wherein the fluorogenic small molecule is conjugated to the lysine at that position.

39. The fluorogenic sensor of any one of claims 34-38, wherein one or more other lysines of the nanobody, other than the lysine conjugated to the fluorogenic small molecule, are independently substituted by a different amino acid.

40. The fluorogenic sensor of claim 39, wherein one or more other lysines of the nanobody, other than the lysine conjugated to the fluorogenic small molecule, are independently substituted by arginine (R).

41. The fluorogenic sensor of any one of claims 1-7 and 34-40, wherein the nanobody comprises an amino acid sequence with at least 80% sequence identity with SEQ ID NO: 13.

42. The fluorogenic sensor of claim 41, wherein the nanobody comprises an amino acid sequence with about 90%, 91%, 92%, 93%, 94%, 95%, 96%, 97%, 98%, 99% or 100% sequence identity with SEQ ID NO: 13.

43. The fluorogenic sensor of claim 42, wherein the nanobody is sdAb-B6 noK K65.

44. The fluorogenic sensor of claim 1 or 2, wherein the nanobody binds an epitope tag.

45. The fluorogenic sensor of any one of claims 1, 2, and 44, wherein the nanobody binds ALFA-tag.

46. The fluorogenic sensor of any one of claims 1, 2, 44, and 45, wherein the nanobody is a NbALFA nanobody.

47. The fluorogenic sensor of any one of claims 1, 2, and 44-46, wherein the nanobody comprises an amino acid sequence with at least 80% sequence identity with SEQ ID NO: 16.

48. The fluorogenic sensor of claim 47, wherein the nanobody comprises an amino acid sequence at least 85%, 90%, 91%, 92%, 93%, 94%, 95%, 96%, 97%, 98%, 99%, or 100% identical to SEQ ID NO: 16.

49. The fluorogenic sensor of any one of claims 44-48, wherein the nanobody comprises one or more amino acids substituted by cysteine (C), wherein the fluorogenic small molecule is conjugated to one of said cysteine.

50. The fluorogenic sensor of any one of claims 47-49, wherein amino acid sequence comprises a M63C substitution; and wherein the fluorogenic small molecule is conjugated to the cysteine at the substituted position.

51. The fluorogenic sensor of any one of claims 1, 2, and 44-50, wherein the nanobody comprises an amino acid sequence with at least 80% sequence identity with SEQ ID NO: 17.

52. The fluorogenic sensor of claim 51, wherein the nanobody comprises an amino acid sequence with about 90%, 91%, 92%, 93%, 94%, 95%, 96%, 97%, 98%, 99% or 100% sequence identity with SEQ ID NO: 17.

53. The fluorogenic sensor of claim 52, wherein the nanobody is NbALFA M63C.

54. The fluorogenic sensor of claim 1 or 2, wherein the nanobody binds a small molecule.

55. The fluorogenic sensor of any one of claims 1, 2, and 54 wherein the nanobody binds cortisol.

56. The fluorogenic sensor of any one of claims 1, 2, 54, and 55, wherein the nanobody is a NbCor nanobody.

57. The fluorogenic sensor of any one of claims 1, 2, and 54-56, wherein the nanobody comprises an amino acid sequence with at least 80% sequence identity with SEQ ID NO: 18.

58. The fluorogenic sensor of claim 57, wherein the nanobody comprises an amino acid sequence at least 85%, 90%, 91%, 92%, 93%, 94%, 95%, 96%, 97%, 98%, or 99% identical to SEQ ID NO: 18.

59. The fluorogenic sensor of any one of claims 54-58, wherein the fluorogenic small molecule is conjugated to a lysine or cysteine residue of nanobody.

60. The fluorogenic sensor of any one of claims 54-59, wherein the nanobody comprises one or more amino acids substituted by lysine (K) or cysteine (C), wherein the fluorogenic small molecule is conjugated to one of said lysine or cysteine.

61. The fluorogenic sensor of any one of claims 57-60, wherein amino acid sequence comprises at least one amino acid substitution selected from T53C, N27C, T28C, S30C, V24C, G29C, and W34C; and wherein the fluorogenic small molecule is conjugated to the cysteine at the substituted position.

62. The fluorogenic sensor of any one of claims 57-60, wherein the amino acid sequence comprises at least one amino acid substitution selected from V24K and A78K; and wherein the fluorogenic small molecule is conjugated to the lysine at the substituted position.

63. The fluorogenic sensor of any one of claims 57-59, wherein the fluorogenic small molecule is conjugated to the lysine at position K79.

64. The fluorogenic sensor of any one of claims 54-63, wherein one or more other lysines of the nanobody, other than the lysine conjugated to the fluorogenic small molecule, are independently substituted by a different amino acid.

65. The fluorogenic sensor of claim 64, wherein one or more other lysines of the nanobody, other than the lysine conjugated to the fluorogenic small molecule, are independently substituted by arginine (R).

66. The fluorogenic sensor of any one of claims 1, 2, and 54-65, wherein the nanobody comprises an amino acid sequence with at least 80% sequence identity with SEQ ID NO: 19.

67. The fluorogenic sensor of claim 66, wherein the nanobody comprises an amino acid sequence with about 90%, 91%, 92%, 93%, 94%, 95%, 96%, 97%, 98%, 99%, or 100% sequence identity with SEQ ID NO: 19.

68. The fluorogenic sensor of claim 67, wherein the nanobody is NbCor T53C.

69. A fluorogenic sensor for detecting a target comprising:
a mini-protein; and
a fluorogenic small molecule conjugated to the mini-protein.

70. The fluorogenic sensor of claim 69, wherein the fluorogenic small molecule is conjugated at or around a target-binding domain of the mini-protein.

71. The fluorogenic sensor of claim 70, wherein the fluorogenic small molecule is conjugated to a target-binding domain of the mini-protein.

72. The fluorogenic sensor of any one of claims 69-71, wherein the mini-protein binds an antigen.

73. The fluorogenic sensor of any one of claims 69-72, wherein the mini-protein binds a pathogen.

74. The fluorogenic sensor of any one of claims 69-73, wherein the mini-protein binds a spike protein of a coronavirus or variant thereof.

75. The fluorogenic sensor of claim 74, wherein the coronavirus is a SARS-CoV-2 virus or variant thereof.

76. The fluorogenic sensor of any one of claim 69-75, wherein the mini-protein is a LCB3 protein.

77. The fluorogenic sensor of any one of claims 69-76, wherein the mini-protein comprises an amino acid sequence with at least 80% sequence identity with SEQ ID NO: 14.

78. The fluorogenic sensor of claim 77, wherein the mini-protein comprises an amino acid sequence at least 85%, 90%, 91%, 92%, 93%, 94%, 95%, 96%, 97%, 98%, 99%, or 100% identical to SEQ ID NO: 14.

79. The fluorogenic sensor of any one of claims 69-78, wherein the mini-protein comprises one or more amino acids substituted by cysteine (C), wherein the fluorogenic small molecule is conjugated to one of said cysteine.

80. The fluorogenic sensor of any one of claims 77-79, wherein amino acid sequence comprises a H19C substitution; and wherein the fluorogenic small molecule is conjugated to the cysteine at the substituted position.

81. The fluorogenic sensor of any one of claims 69-80, wherein the mini-protein comprises an amino acid sequence with at least 80% sequence identity with SEQ ID NO: 15.

82. The fluorogenic sensor of claim 81, wherein the mini-protein comprises an amino acid sequence with about 90%, 91%, 92%, 93%, 94%, 95%, 96%, 97%, 98%, 99% or 100% sequence identity with SEQ ID NO: 15.

83. The fluorogenic sensor of claim 82, wherein the mini-protein is LCB3 H19C.

84. The fluorogenic sensor of any one of claims 1-83, wherein the fluorogenic small molecule conjugated to the nanobody or the mini-protein results from conjugating a compound of the following formula: FG-L-A, or a salt, stereoisomer, or tautomer thereof; wherein FG is the fluorogenic small molecule; L is a bond or a linker; and A is a reactive moiety.

85. The fluorogenic sensor of claim 84, wherein A is a lysine- or cysteine-selective reactive moiety.

86. The fluorogenic sensor of claim 84 or 85, wherein FG is of one of the following formulae: 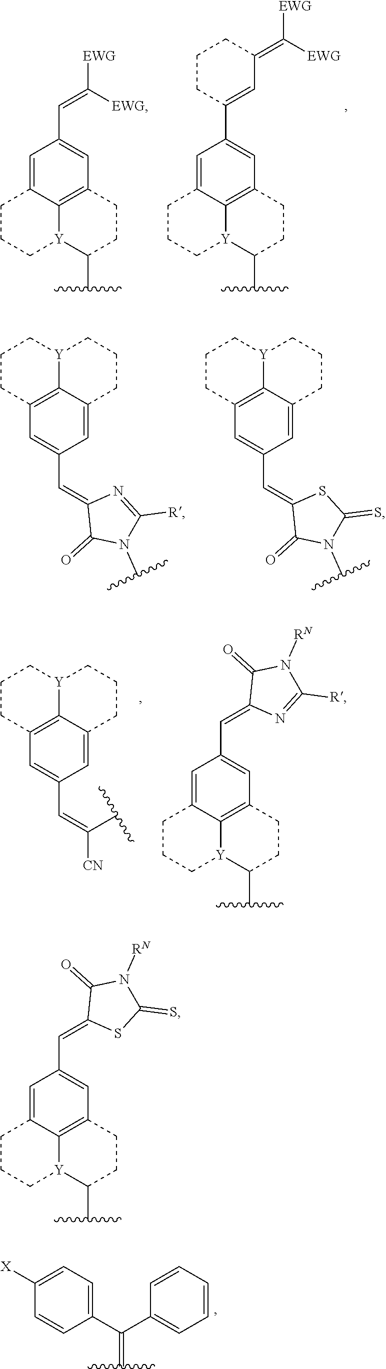 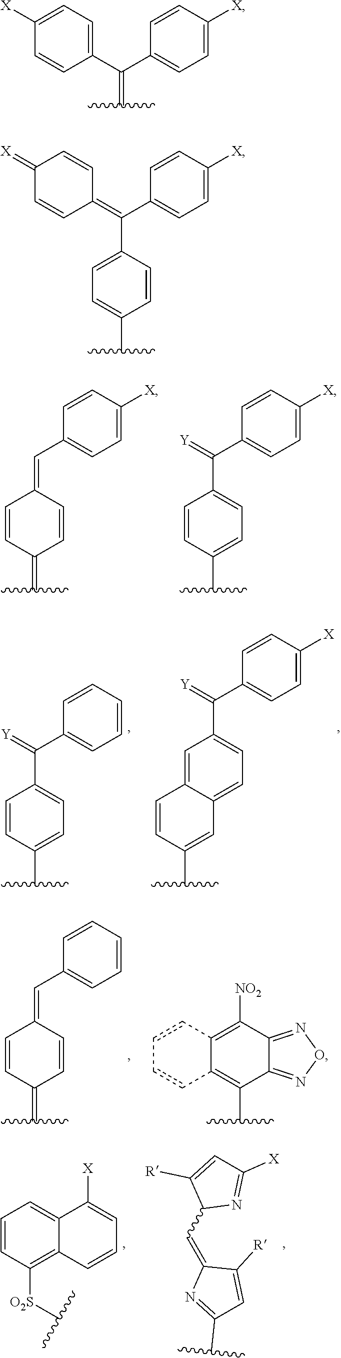
or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

87. The fluorogenic sensor of any one of claims 84-86, wherein -L-A is of one of the following formulae: 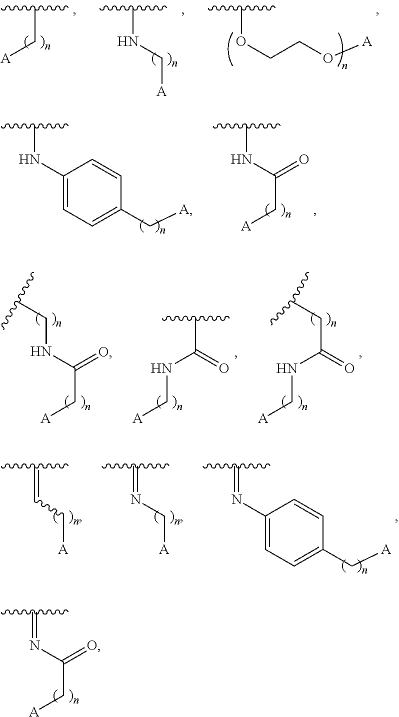
or a salt, stereoisomer, or tautomer thereof; wherein:
each n is independently 0 or an integer from 1-20, inclusive; and
wherein each formula is further optionally substituted.

88. The fluorogenic sensor of any one of claims 84-87, wherein A comprises a halogen, alkene, alkyne, azide, tetrazine, or a moiety of one of the following formulae: 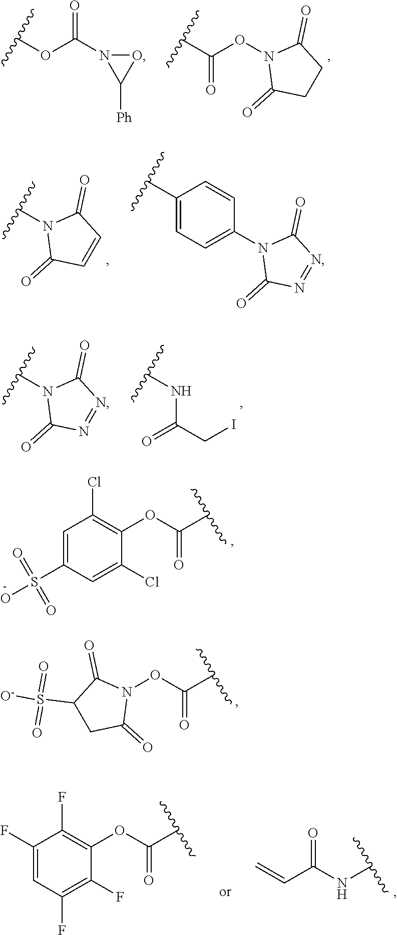
or a salt, stereoisomer, or tautomer thereof; wherein each formula is further optionally substituted.

89. The fluorogenic sensor of any one of the preceding claims, wherein the the nanobody or the mini-protein comprises an unnatural amino acid comprising the fluorogenic small molecule; optionally wherein a target-binding domain of the nanobody or the mini-protein comprises an unnatural amino acid comprising the fluorogenic small molecule.

90. The fluorogenic sensor of any one of the preceding claims, wherein the amino acid sequence comprises at least one amino acid substituted by an unnatural amino acid comprising the fluorogenic small molecule.

91. The fluorogenic sensor of claim 89 or 90, wherein the unnatural amino acid comprising the fluorogenic small molecule is encoded into the amino acid sequence or installed via transpeptidation.

92. The fluorogenic sensor of claim 89 or 90, wherein the unnatural amino acid comprising the fluorogenic small molecule is encoded into the amino acid sequence via ribosomal synthesis.

93. The fluorogenic sensor of any one of claims 89-92, wherein the unnatural amino acid comprising the fluorogenic small molecule is of one of the following formulae: 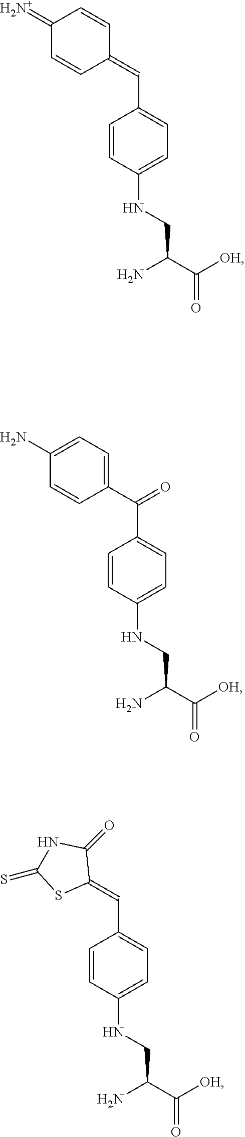 [SMILES: N[C@@H](CNc1ccc(C=C2C=CC(=[NH2+])C=C2)cc1)C(=O)O ; Nc1ccc(C(=O)c2ccc(NC[C@H](N)C(=O)O)cc2)cc1 ; N[C@@H](CNc1ccc(/C=C2\SC(=S)NC2=O)cc1)C(=O)O] 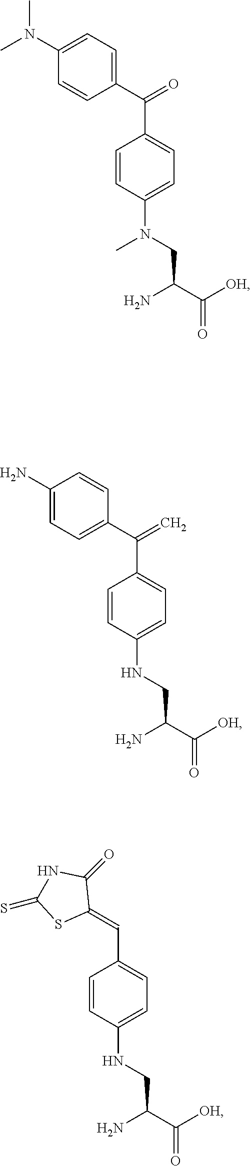 [SMILES: CN(C)c1ccc(C(=O)c2ccc(N(C)C[C@H](N)C(=O)O)cc2)cc1 ; C=C(c1ccc(N)cc1)c1ccc(NC[C@H](N)C(=O)O)cc1 ; N[C@@H](CNc1ccc(/C=C2\SC(=S)NC2=O)cc1)C(=O)O] 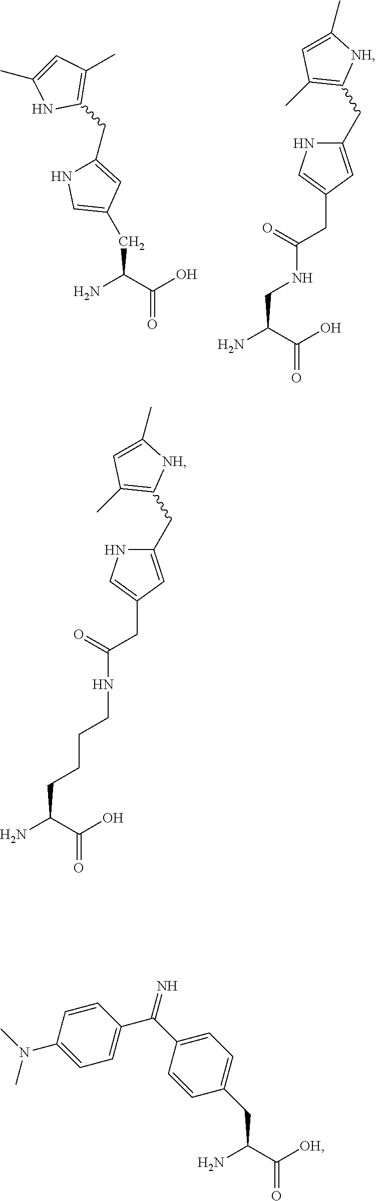 [SMILES: Cc1cc(C)c(Cc2cc(C[C@H](N)C(=O)O)c[nH]2)[nH]1 ; Cc1cc(C)c(Cc2cc(CC(=O)NC[C@H](N)C(=O)O)c[nH]2)[nH]1 ; Cc1cc(C)c(Cc2cc(CC(=O)NCCCC[C@H](N)C(=O)O)c[nH]2)[nH]1 ; CN(C)c1ccc(C(=N)c2ccc(C[C@H](N)C(=O)O)cc2)cc1] 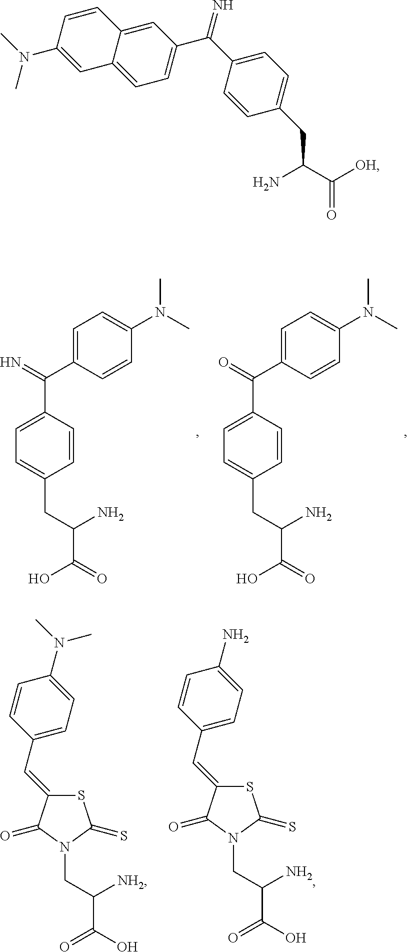 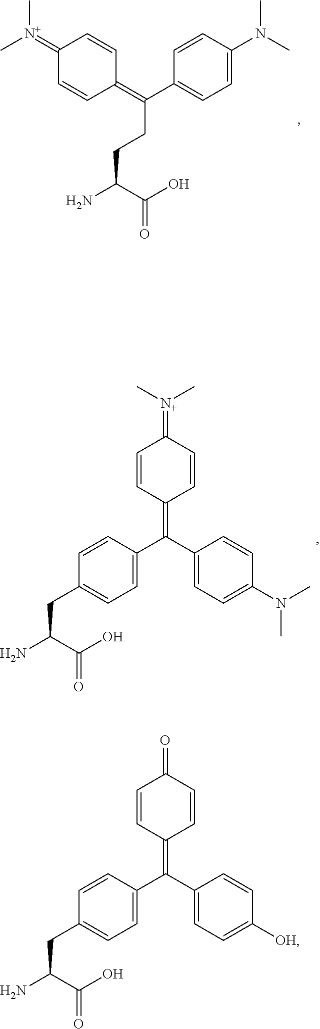 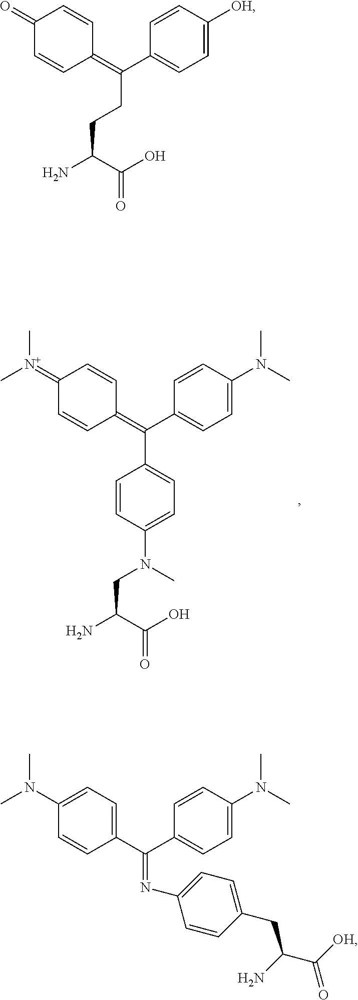
or a salt, stereoisomer, or tautomer thereof.

94. The fluorogenic sensor of any one of the preceding claims, wherein the fluorogenic small molecule is 7-nitrobenz-2-Oxa-1,3-Diazol-4-yl (NBD).

95. The fluorogenic sensor of any one of the preceding claims, wherein the fluorogenic small molecule conjugated to the nanobody or the mini-protein results from conjugating a compound selected from those in FIG. 2A and FIG. 2B.

96. The fluorogenic sensor of any one of the preceding claims, wherein the fluorescence of the fluorogenic small molecule increases upon binding of the nanobody or the mini-protein to the target.

97. The fluorogenic sensor of any one of the preceding claims, wherein the fluorescence lifetime of the fluorogenic small molecule changes upon binding of the nanobody or the mini-protein to the target.

98. The fluorogenic sensor of any one of the preceding claims further comprising a second fluorogenic small molecule conjugated to the N-terminus of the nanobody.

99. The fluorogenic sensor of claim 98, wherein the first fluorogenic small molecule and the second fluorogenic small molecule are a fluorescence resonance energy transfer (FRET) pair.

100. The fluorogenic sensor of 98 or 99, wherein the second fluorogenic small molecule is tetramethylrhodamine (TMR).

101. A method of detecting a target, the method comprising:
(i) contacting a target with a fluorogenic sensor of any one of the preceding claims; and
(ii.a) measuring or observing the fluorescence of the fluorogenic sensor, or (ii.b) measuring or observing a change in the fluorescence lifetime of the fluorogenic sensor.

102. A method of detecting an antigen, the method comprising:
(i) contacting an antigen with a fluorogenic sensor of any one of the preceding claims; and
(ii.a) measuring or observing the fluorescence of the fluorogenic sensor, or (ii.b) measuring or observing a change in the fluorescence lifetime of the fluorogenic sensor.

103. The method of claim 102, wherein the antigen is pathogen.

104. The method of claim 103, wherein the pathogen is a coronavirus or variant thereof.

105. The method of claim 104, wherein the coronavirus is SARS-CoV-2 or variant thereof.

106. The method of claim 101, wherein the target is an ALFA-tagged protein.

107. The method of claim 106, wherein the target is a bacterial cell expressing an ALFA-tagged protein.

108. The method of claim 101, wherein the target is cortisol.

109. The method of any one of claims 101-108, wherein a change in fluorescence and/or fluorescence lifetime is observed instantaneously after the contacting step.

110. The method of claim 109, wherein the change in fluorescence and/or fluorescence lifetime is observed within less than 1 second after the contacting step.

111. The method of claim 110, wherein change in fluorescence and/or fluorescence lifetime is observed within less than less than 2500, 2000, 1500, 1000, 750, 500, or 250 milliseconds (ms) after the contacting step.

112. The method of any one of claims 101-111, wherein an increase in fluorescence of at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold is observed.

113. A compound of the following formula: 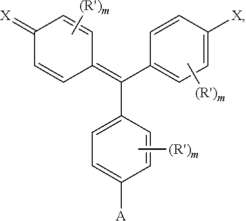
or a salt, stereoisomer, or tautomer thereof, wherein:
A is a reactive moiety comprising one of the following formulae: 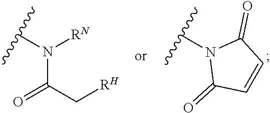
R^{H} is halogen or a leaving group;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

114. The compound of claim 113, wherein the compound is selected from the group consisting of: 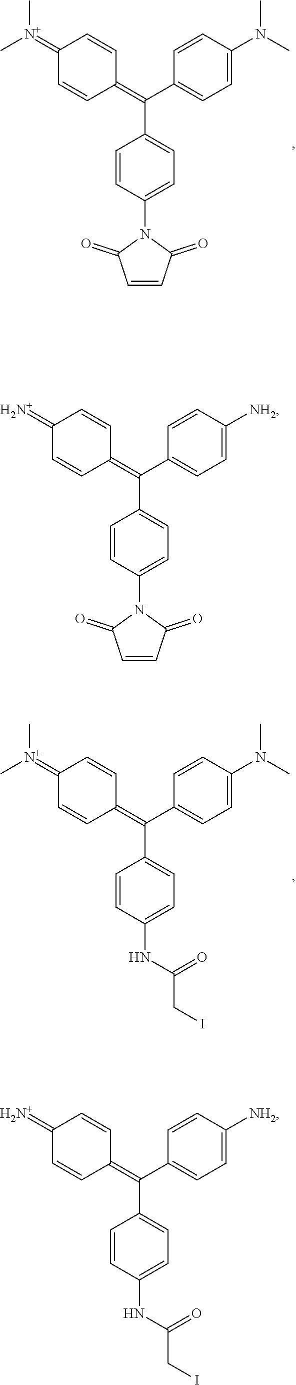
and salts, stereoisomers, and tautomers thereof.

115. A compound of the following formula: 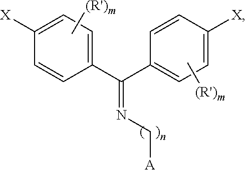
or a salt or tautomer thereof, wherein:
A is a reactive moiety;
each instance of X is independently —N(R^{N})₂, —OR^{O}, or —SR^{S};
n is 0 or an integer from 1-20, inclusive.
each instance of R′ is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl;
each instance of m is independently 0, 1, 2, 3, or 4;
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

116. The compound of claim 115, wherein A comprises a halogen, alkyne, azide, or a moiety of one of the following formulae: 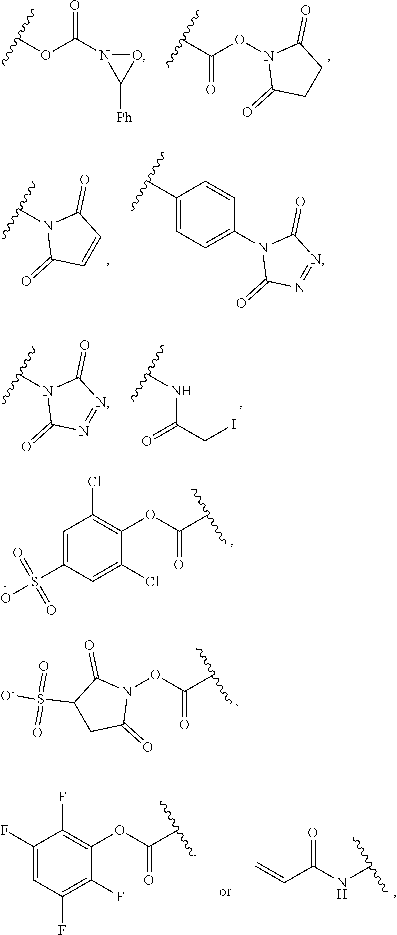
or a salt, stereoisomer, or tautomer thereof; wherein each formula is further optionally substituted.

117. The compound of claim 116, wherein the compound is of the formula: 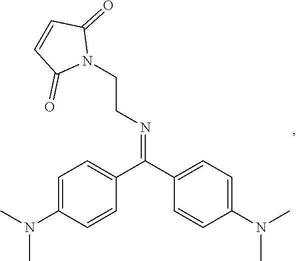
or a salt or tautomer thereof.

118. A compound of the following formula: 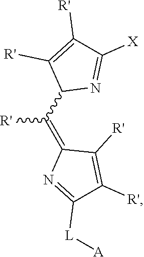
or a salt, stereoisomer, or tautomer thereof; wherein:
L is a bond or a linker;
A is a reactive moiety;
each instance of R′ and X is independently hydrogen, halogen, —CN, —NO₂, —N₃, —N(R^{N})₂, —OR^{O}, —SR^{S}, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, or optionally substituted sulfonyl; and
each instance of R^{N}, R^{O}, and R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, or optionally substituted acyl.

119. The compound of claim 118, wherein the compound is of the following formula: 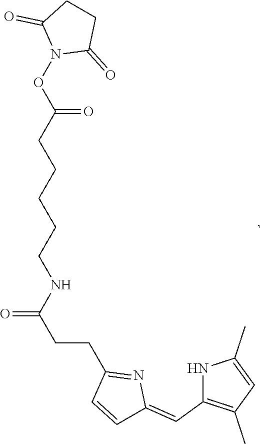 [SMILES: Cc1cc(C)c(/C=C2/C=CC(CCC(=O)NCCCCCC(=O)ON3C(=O)CCC3=O)=N2)[nH]1]
or a salt or tautomer thereof.

120. A compound of the following formula: 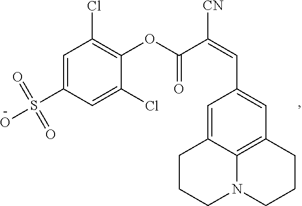 [SMILES: N#C/C(=C/c1cc2c3c(c1)CCCN3CCC2)C(=O)Oc1c(Cl)cc(S(=O)(=O)O)cc1Cl]
or a salt, stereoisomer, or tautomer thereof; wherein the compound is further optionally substituted.

121. A kit comprising a fluorogenic sensor or compound of any one of the preceding claims, and optionally instructions for use.
